(19) United States
(12) Patent Application Publication
(10) Pub. No.: US 2011/0136835 A1
(43) Pub. Date: Jun. 9, 2011

(54) COMBINATION FORMULATIONS OF TRANILAST AND ALLOPURINOL AND METHODS RELATED THERETO

(75) Inventors: Michael Kitt, Mountain View, CA (US); Tito Serafini, Belmont, CA (US)

(73) Assignee: NUON THERAPEUTICS, INC., San Mateo, CA (US)

(21) Appl. No.: 12/882,143

(22) Filed: Sep. 14, 2010

Related U.S. Application Data
(63) Continuation-in-part of application No. PCT/US2009/068883, filed on Dec. 19, 2009.
Application No. PCT/US2009/068883 is a continuation-in-part of application No. 12/642,802, filed on Dec. 19, 2009.
(60) Provisional application No. 61/242,354, filed on Sep. 14, 2009.
(60) Provisional application No. 61/242,344, filed on Sep. 14, 2009.
(60) Provisional application No. 61/354,655, filed on Jun. 14, 2010.

(51) Int. Cl. A61K 31/519; A61P 19/06

(52) U.S. Cl. 514/262.1

(57) ABSTRACT

Disclosed is a pharmaceutical composition comprising tranilast or a pharmaceutically acceptable salt thereof and allopurinol or a pharmaceutically acceptable salt thereof, wherein the amount by weight of said allopurinol or pharmaceutically acceptable salt thereof in said composition is greater than the amount by weight of said tranilast or pharmaceutically acceptable salt thereof in said composition.

### CROSS-REFERENCE TO RELATED APPLICATIONS

This application claims the benefit of priority under 35 U.S.C. §119(e) of U.S. Provisional Application Ser. Nos. 61/242,354, filed Sep. 14, 2009; 61/242,344, filed Sep. 14, 2009; and 61/354,655, filed Jun. 14, 2010. Additionally, this application is a continuation-in-part of International Application No. PCT/US2009/068883, filed Dec. 19, 2009; and is a continuation-in-part of U.S. Utility application Ser. No. 12/642,802, filed Dec. 19, 2009. Both International Application No. PCT/US2009/068883 and U.S. Utility application Ser. No. 12/642,802 claim the benefit of priority under 35 U.S.C. 119(e) of U.S. Provisional Application Ser. Nos. 61/242,354; 61/242,344; 61/139,415, filed Dec. 19, 2008; 61/140,802, filed Dec. 24, 2008; 61/161,754, filed Mar. 19, 2009; and 61/165,114, filed Mar. 31, 2009. The contents of above-mentioned applications are hereby incorporated by reference in their entireties for all purposes.

### BACKGROUND OF THE INVENTION

Gout, which is sometimes called podagre, affects 3 to 5 million individuals in the United States and continues to increase in incidence. Gout includes a group of disorders including painful attacks of acute, monarticular, inflammatory arthritis due to uric acid crystals, deposition of urate crystals in joints, deposition of urate crystals in renal parenchyma, urolithiasis (formation of calculus in the urinary tract), and nephrolithiasis (formation of kidney stones). Gouty arthritis is usually an extremely painful attack of gout with a rapid onset of joint inflammation. The joint inflammation is precipitated by deposits of uric-acid crystals in the joint fluid (synovial fluid) and joint lining (synovial lining). Intense joint inflammation occurs as white blood cells engulf the uric-acid crystals and release chemicals of inflammation, causing pain, heat, and redness of the joint tissues. Chronic gout can lead to deposits of hard lumps of uric acid in and around the joints, kidney stones, and blockage of the kidney-filtering tubules with uric-acid crystals, leading to kidney failure.

The underlying metabolic aberration in gout is hyperuricemia. Hyperuricemia has been associated with a serum uric acid (sUA) level of 6.8 mg/dL or greater, which is the upper limit of solubility of uric acid (also called urate) extracellular fluids. However, hyperuricemia also has been associated with other levels of serum uric acid depending on factors such as gender and age, for example. Hyperuricemia leads to gout when urate crystals are formed from supersaturated body fluids and deposited in joints, tophi, and parenchymal organs.

In addition to gout, other disorders related to elevated serum uric acid levels include gout-associated inflammation, renal disorders, cardiovascular disease, aberrant metabolic conditions, fatty liver disease, kidney stones, cognitive impairment and dementia. Elevated serum uric acid levels have been identified as an independent risk factor for chronic kidney disease, cardiovascular disease and hypertension. Edwards, N. L., Clev. Clin. J. Med., Vol. 75, Suppl. 5, July 2008, S13-16. With respect to cardiovascular disease, hyperuricemia has been identified as an independent risk factor for atherosclerotic disease in general and for coronary artery disease in particular. Edwards, N. L., Curr. Opin. Rheum., 2009, 21:132-137. It also has been identified as arm independent risk factor for heart failure. Krishnan, E., Circ. Heart Fail., 2009, 2:556-562. Hyperuricemia independent of crystal deposition also may play a pathogenetic role in aberrant metabolic states, such as hypertriglyceridemia, obesity, insulin resistance, diabetes and metabolic syndrome. Beck, M. A., et. al, Rheum Dis. Clin. Am., 32 (2006), 275-293. In addition, hyperuricemia has been linked to cerebral vascular disease. Edwards, N. L., Curr. Opin. Rheum., 2009, 21:132-137. Elevated serum uric acid also has been independently associated with non-alcoholic fatty liver disease (NAFLD). Lee, Y-J., et. al, Clin. Chem. Lab. Med., 2010, 48(2). Elevated serum uric acid also has been identified as a strong risk factor for myocardial infarction and stroke. Bos, M. J., et al., Stroke, 2006, 37, 1503-1507. In addition, normal but mildly elevated serum uric acid levels have been linked to cognitive impairment and dementia. Schretlen, D. J., et al., Neuropsychology, 2007, Vol. 21, No. 1, 136-140.

The biosynthesis pathway for uric acid is represent in the following Scheme I:

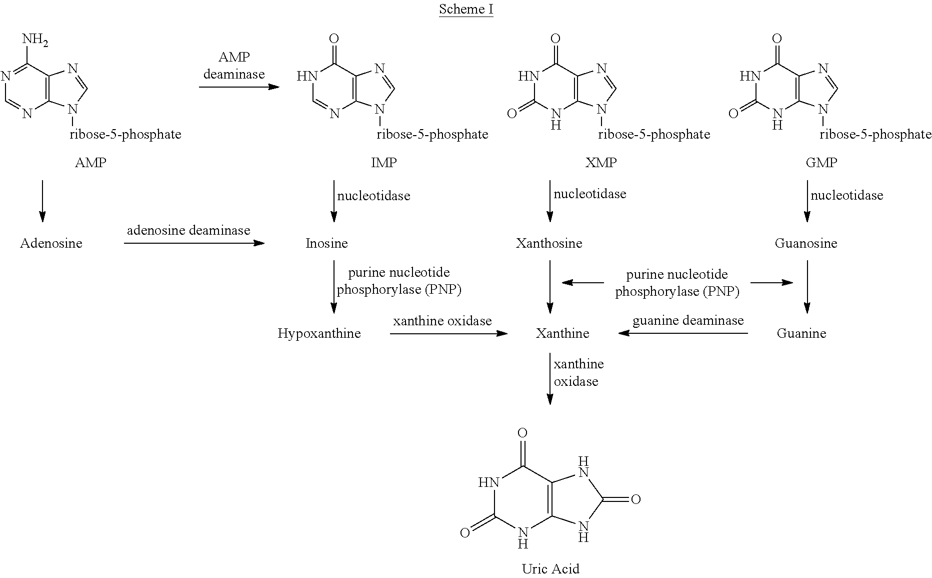

Scheme I. Purines are converted to hypoxanthine, then xanthine and finally urate via sequential oxidation by the enzyme xanthine oxidase.

Hyperuricemia can result from increased production or decreased excretion of uric acid, or from a combination of the two processes. Urate is the end product of purine metabolism in humans, shown above in Scheme I.

Known methods for treating gout include the use of uric acid synthesis inhibitors to inhibit the accumulation of uric acid in the body. These compounds function by inhibiting an enzyme involved in uric acid synthesis. In fact, it may be possible to inhibit uric acid synthesis by inhibiting any one of several enzymes shown above to be involved in uric acid synthesis. For example, xanthine oxidase inhibitors, such as febuxostat and allopurinol or a pharmaceutically acceptable salt thereof, reduce serum uric acid levels by inhibiting the enzyme xanthine oxidase. Known methods also include introduction of a recombinant, non-human uricase enzyme into the body, such as rasburicase or pegloticase.

Another known method for treating gout involves the use of uric acid excretion promoters These compounds accelerate the rapid excretion of uric acid accumulated in the body. Probenecid, sulfinpyrazone and benzbromarone are examples of uric acid excretion promoters. These compounds prevent the reuptake of urate back into the bloodstream in the kidney, leading to a net increase in excretion. Interleukin-6 (IL-6) has been proposed for use in the treatment of gout as a serum uric acid decreasing agent (see U.S. Pat. No. 6,007,804). In addition, non-steroidal anti-inflammatory drugs (NSAIDs), corticosteroids and colchicine have been used to treat some of the painful symptoms associated with gout.

However, the previously used anti-hyperuricemia agents all have different side effects or toxicity, such as the deposition of urate crystal in the urethra, leading to renal dysfunction and renal colic.

Therefore, it is necessary to find new pharmaceutical compositions and methods for treating gout, hyperuricemia and related disorders, and for lowering serum uric acid levels.

### SUMMARY OF THE INVENTION

In one aspect the present invention is directed to a pharmaceutical composition comprising tranilast or a pharmaceutically acceptable salt thereof and allopurinol or a pharmaceutically acceptable salt thereof, wherein the amount by weight of said allopurinol or pharmaceutically acceptable salt thereof in said composition is greater than the amount by weight of said tranilast or pharmaceutically acceptable salt thereof in said composition.

In another aspect the present invention provides a method of treating a condition associated with an elevated serum uric acid level comprising administering to a subject in need thereof tranilast or a pharmaceutically acceptable salt thereof and allopurinol or a pharmaceutically acceptable salt thereof, wherein the amount, by weight, of allopurinol or pharmaceutically acceptable salt thereof administered is greater than the amount, by weight, of tranilast or pharmaceutically acceptable salt thereof administered.

In another aspect the present invention provides a method of decreasing serum uric acid level in a subject having a condition associated with an elevated serum uric acid level comprising administering to a subject in need thereof tranilast or a pharmaceutically acceptable salt thereof and allopurinol or a pharmaceutically acceptable salt thereof, wherein the amount, by weight, of allopurinol or pharmaceutically acceptable salt thereof administered is greater than the amount, by weight, of tranilast or pharmaceutically acceptable salt thereof administered.

Another aspect of the present invention provides a method of decreasing serum uric acid level in a subject comprising administering to a subject in need thereof tranilast or a pharmaceutically acceptable salt thereof and allopurinol or a pharmaceutically acceptable salt thereof, wherein the amount, by weight, of allopurinol or pharmaceutically acceptable salt thereof administered is greater than the amount, by weight, of tranilast or pharmaceutically acceptable salt thereof administered.

Another aspect of the present invention provides a method of treating hyperuricemia in a subject comprising administering to a subject in need thereof tranilast or a pharmaceutically acceptable salt thereof and allopurinol or a pharmaceutically acceptable salt thereof, wherein the amount, by weight, of allopurinol or pharmaceutically acceptable salt thereof administered is greater than the amount, by weight, of tranilast or pharmaceutically acceptable salt thereof administered.

In another aspect the present invention provides a method of treating gout in a subject comprising administering to a subject in need thereof tranilast or a pharmaceutically acceptable salt thereof and allopurinol or a pharmaceutically acceptable salt thereof, wherein the amount, by weight, of allopurinol or pharmaceutically acceptable salt thereof administered is greater than the amount, by weight, of tranilast or pharmaceutically acceptable salt thereof administered.

In another aspect the present invention provides a method for increasing the serum uric acid lowering effectiveness of allopurinol or a pharmaceutically acceptable salt thereof in a subject whose reduction in serum uric acid level upon administration of allopurinol or a pharmaceutically acceptable salt thereof is less than the median response of subjects administered an equivalent amount of allopurinol or pharmaceutically acceptable salt thereof, comprising administering to a subject in need thereof tranilast or a pharmaceutically acceptable salt thereof and allopurinol or a pharmaceutically acceptable salt thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawing of which:

FIG. 1 shows the effects of Tranilast on serum uric acid levels in hyperuricemic patients. All patients had uric acid baseline levels equal to or above 8 mg/dL. Tranilast of was administered twice daily for one or three months at the indicated dosages.

FIG. 2 shows the percent change from baseline in serum uric acid levels of upon administration of 400 mg of allopurinol as compared to administration of 300 mg of tranilast and 400 mg of allopurinol or a pharmaceutically acceptable salt thereof.

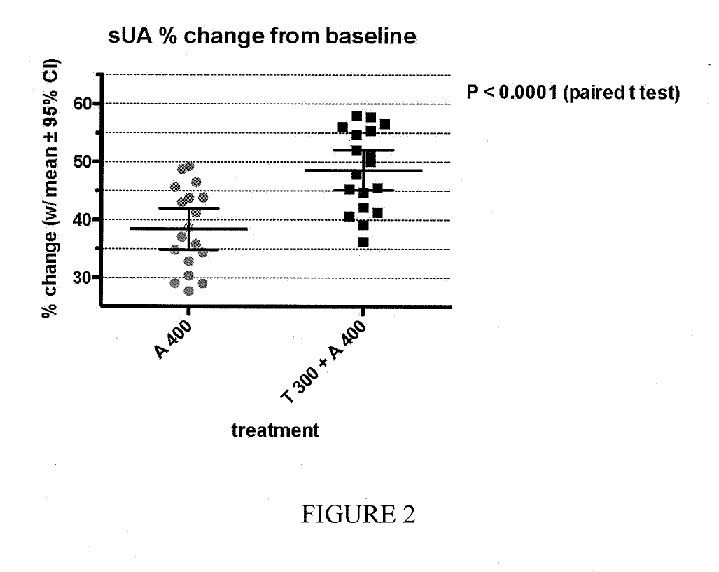

FIG. 3 shows the percent change from baseline in serum uric acid levels of individual subjects upon administration of 400 mg of allopurinol as compared to administration of 300 mg of tranilast and 400 mg of allopurinol.

FIG. 4 shows the percent of subjects in individual dosing groups with <4 mg/dL sUA levels after treatment with (1) Allopurinol 300 mg, (2) Allopurinol 400 mg, (3) Allopurinol 300 mg+Tranilast 300 mg or (4) Allopurinol 400 mg+Tranilast 300 mg.

FIG. 5 is a flowchart of the phase 2 study design.

### DETAILED DESCRIPTION OF THE INVENTION

Surprisingly, Applicants have discovered that administration of tranilast or a pharmaceutically acceptable salt thereof and allopurinol or a pharmaceutically acceptable salt thereof to a subject, wherein the amount by weight of allopurinol or a pharmaceutically acceptable salt thereof administered is greater than the amount by weight of tranilast or a pharmaceutically acceptable salt thereof administered significantly increases, as compared to administration of an equal amount by weight of tranilast and allopurinol, the percentage of subjects whose serum uric acid levels are reduced to below 4.0 mg/dL. Specifically, Applicants have discovered, surprisingly, that administration of 300 mg of tranilast in combination with 400 mg of allopurinol significantly increases the percentage of subjects whose serum uric acid levels are reduced to below 4.0 mg/dL. Applicants believe this effect will be observed upon administration of 300 mg of tranilast in combination with doses of allopurinol above 400 mg (e.g., 600 mg, 800 mg or 900 mg).

In one embodiment the present invention is directed to a pharmaceutical composition comprising tranilast or a pharmaceutically acceptable salt thereof and allopurinol or a pharmaceutically acceptable salt thereof, wherein the amount by weight of allopurinol or a pharmaceutically acceptable salt thereof in the composition is greater than the amount by weight of tranilast or a pharmaceutically acceptable salt thereof in the composition. In one such embodiment the weight ratio of allopurinol or pharmaceutically acceptable salt thereof to tranilast or pharmaceutically acceptable salt thereof is at least 3.5:3. In another such embodiment the weight ratio of allopurinol or pharmaceutically acceptable salt thereof to tranilast or pharmaceutically acceptable salt thereof is at least 4:3. In another such embodiment the weight ratio of allopurinol or pharmaceutically acceptable salt thereof to tranilast or pharmaceutically acceptable salt thereof is at least 5:3. In another such embodiment the weight ratio of allopurinol or pharmaceutically acceptable salt thereof to tranilast or pharmaceutically acceptable salt thereof is at least 2:1. In another such embodiment the weight ratio of allopurinol or pharmaceutically acceptable salt thereof to tranilast or pharmaceutically acceptable salt thereof is at least 7:3. In another such embodiment the weight ratio of allopurinol or pharmaceutically acceptable salt thereof to tranilast or pharmaceutically acceptable salt thereof is at least 8:3. In another embodiment, the weight ratio of allopurinol or a pharmaceutically acceptable salt thereof to tranilast or a pharmaceutically acceptable salt thereof is at least 3:1 (e.g., 900 mg allopurinol with 300 mg tranilast, 250 mg tranilast, 200 mg tranilast or 150 mg tranilast).

In another such embodiment the weight ratio of allopurinol or pharmaceutically acceptable salt thereof to tranilast or pharmaceutically acceptable salt thereof is about 4:3. In another such embodiment the weight ratio of allopurinol or pharmaceutically acceptable salt thereof to tranilast or pharmaceutically acceptable salt thereof is about 2:1 or about 3:1.

In another embodiment the present invention provides a method of treating a condition associated with an elevated serum uric acid level comprising administering to a subject in need thereof tranilast or a pharmaceutically acceptable salt thereof and allopurinol or a pharmaceutically acceptable salt thereof, wherein the amount, by weight, of allopurinol or a pharmaceutically acceptable salt thereof administered is greater than the amount, by weight, of tranilast or a pharmaceutically acceptable salt thereof administered.

In another embodiment the present invention provides a method of decreasing serum uric acid level in a subject having a condition associated with an elevated serum uric acid level comprising administering to a subject in need thereof tranilast or a pharmaceutically acceptable salt thereof and allopurinol or a pharmaceutically acceptable salt thereof, wherein the amount, by weight, of allopurinol or a pharmaceutically acceptable salt thereof administered is greater than the amount, by weight, of tranilast or a pharmaceutically acceptable salt thereof administered.

In yet another embodiment the present invention provides a method of decreasing serum uric acid level in a subject comprising administering to a subject in need thereof tranilast or a pharmaceutically acceptable salt thereof and allopurinol or a pharmaceutically acceptable salt thereof, wherein the amount, by weight, of allopurinol or a pharmaceutically acceptable salt thereof administered is greater than the amount, by weight, of tranilast or a pharmaceutically acceptable salt thereof administered.

In yet another embodiment the present invention provides a method of treating hypericemia in a subject comprising administering to a subject in need thereof tranilast or a pharmaceutically acceptable salt thereof and allopurinol or a pharmaceutically acceptable salt thereof, wherein the amount, by weight, of allopurinol or a pharmaceutically acceptable salt thereof administered is greater than the amount, by weight, of tranilast or a pharmaceutically acceptable salt thereof administered.

In yet another embodiment the present invention provides a method of treating gout in a subject comprising administering to a subject in need thereof tranilast or a pharmaceutically acceptable salt thereof and allopurinol or a pharmaceutically acceptable salt thereof, wherein the amount, by weight, of allopurinol or a pharmaceutically acceptable salt thereof administered is greater than the amount, by weight, of tranilast or a pharmaceutically acceptable salt thereof administered.

In yet another embodiment the present invention provides a method for increasing the serum uric acid lowering effectiveness of allopurinol or a pharmaceutically acceptable salt thereof in a subject whose reduction in serum uric acid level upon administration of allopurinol is less than the median response of subjects administered an equivalent amount of allopurinol, comprising administering to a subject in need thereof tranilast or a pharmaceutically acceptable salt thereof and allopurinol or a pharmaceutically acceptable salt thereof, wherein the amount, by weight, of allopurinol or pharmaceutically acceptable salt thereof administered is greater than the amount, by weight, of tranilast or pharmaceutically acceptable salt thereof administered.

In one embodiment of the present invention, a method of treating hyperuricemia in a subject in need thereof comprising administration of tranilast or a pharmaceutically acceptable salt thereof and allopurinol or a pharmaceutically acceptable salt thereof, wherein the amount by weight of said allopurinol or pharmaceutically acceptable salt thereof is greater than the amount by weight of said tranilast or pharmaceutically acceptable salt thereof, wherein said subject's reduction in serum uric acid level upon administration of allopurinol alone is less than the median response. In another such embodiment, said subject has gout. In another such embodiment, said subject has moderate or severe gout. In another such embodiment, said subject has chronic gout. In another such embodiment, said subject has acute gout. In another such embodiment, said subject has moderate or severe gout, and the amount of tranilast or pharmaceutically acceptable salt thereof administered is from about 300 mg to about 600 mg per day, and the amount of allopurinol administered is from about 350 mg to about 900 mg per day. In another such embodiment, the amount of tranilast or pharmaceutically acceptable salt thereof is about 300 mg and the amount of allopurinol or pharmaceutically acceptable salt thereof is selected from the group consisting of about 350 mg, 400 mg, 500 mg, 600 mg, 700 mg, 800 mg, and 900 mg. In another such embodiment, the amount of tranilast or pharmaceutically acceptable salt thereof is about 300 mg and the amount of allopurinol or pharmaceutically acceptable salt thereof is selected from the group consisting of about 400 mg, 600 mg, and 800 mg. Inn another such embodiment, the amount of tranilast or pharmaceutically acceptable salt thereof is about 300 mg and the amount of allopurinol or pharmaceutically acceptable salt thereof is selected from the group consisting of about 400 mg.

In one embodiment of the present invention, a method of treating hyperuricemia in a subject in need thereof comprising administration of tranilast or a pharmaceutically acceptable salt thereof and allopurinol or a pharmaceutically acceptable salt thereof, wherein the amount by weight of said allopurinol or pharmaceutically acceptable salt thereof is greater than the amount by weight of said tranilast or pharmaceutically acceptable salt thereof, wherein said subject has experienced insufficient lowering of serum uric acid following treatment with allopurinol, febuxostat, probenecid, or pharmaceutically acceptable salts thereof. In another such embodiment, the subject experienced insufficient lowering of serum uric acid following treatment with allopurinol or pharmaceutically acceptable salts thereof. In another such embodiment, said subject has gout. In another such embodiment, said subject has moderate or severe gout. In another such embodiment, said subject has chronic gout. In another such embodiment, said subject has acute gout. In another such embodiment, said subject has moderate or severe gout, and the amount of tranilast or pharmaceutically acceptable salt thereof administered is from about 300 mg to about 600 mg per day, and the amount of allopurinol administered is from about 350 mg to about 900 mg per day. In another such embodiment, the amount of tranilast or pharmaceutically acceptable salt thereof is about 300 mg and the amount of allopurinol or pharmaceutically acceptable salt thereof is selected from the group consisting of about 350 mg, 400 mg, 500 mg, 600 mg, 700 mg, 800 mg, and 900 mg. In another such embodiment, the amount of tranilast or pharmaceutically acceptable salt thereof is about 300 mg and the amount of allopurinol or pharmaceutically acceptable salt thereof is selected from the group consisting of about 400 mg, 600 mg, and 800 mg. In another such embodiment, the amount of tranilast or pharmaceutically acceptable salt thereof is about 300 mg and the amount of allopurinol or pharmaceutically acceptable salt thereof is selected from the group consisting of about 400 mg.

In the methods of the present invention, the amount of tranilast or a pharmaceutically acceptable salt thereof administered to a subject may be from about 100 mg to about 500 mg per day and the amount of allopurinol or pharmaceutically acceptable salt thereof may be from about 150 mg to about 900 mg per day, from about 150 mg to about 800 mg per day, from about 200 mg to about 800 mg per day, from about 350 to 900 mg per day, from about 400 mg to about 800 mg per day, from about 400 mg to about 600 mg per day. In one such embodiment the amount of tranilast or a pharmaceutically acceptable salt thereof administered is about 100 mg, about 200 mg, about 300 mg, about 400 mg or about 500 mg per day. In one such embodiment the amount of tranilast or a pharmaceutically acceptable salt thereof administered is about 100 mg per day. In another such embodiment the amount of tranilast or a pharmaceutically acceptable salt thereof administered is about 200 mg per day. In another such embodiment the amount of tranilast or a pharmaceutically acceptable salt thereof administered is about 300 mg per day. In another such embodiment the amount of tranilast or a pharmaceutically acceptable salt thereof administered is about 400 mg per day. In another such embodiment the amount of tranilast or a pharmaceutically acceptable salt thereof administered is about 500 mg per day.

the methods of the present invention, the amount of allopurinol or a pharmaceutically acceptable salt thereof administered to a subject is greater than the amount of tranilast or a pharmaceutically acceptable salt thereof administered to the subject, and may be from about 150 mg to about 800 mg per day. In one such embodiment, the amount of allopurinol or a pharmaceutically acceptable salt thereof administered to the subject is about 150 mg per day. In another such embodiment, the amount of allopurinol or a pharmaceutically acceptable salt thereof administered to the subject is about 200 mg per day. In another such embodiment, the amount of allopurinol or a pharmaceutically acceptable salt thereof administered to the subject is about 250 mg per day. In another such embodiment, the amount of allopurinol or a pharmaceutically acceptable salt thereof administered to the subject is about 300 mg per day. In another such embodiment, the amount of allopurinol or a pharmaceutically acceptable salt thereof administered to the subject is about 350 mg per day. In another such embodiment, the amount of allopurinol or a pharmaceutically acceptable salt thereof administered to the subject is about 400 mg per day. In another such embodiment, the amount of allopurinol or a pharmaceutically acceptable salt thereof administered to the subject is about 450 mg per day. In another such embodiment, the amount of allopurinol or a pharmaceutically acceptable salt thereof administered to the subject is about 550 mg per day. In another such embodiment, the amount of allopurinol or a pharmaceutically acceptable salt thereof administered to the subject is about 600 mg per day. In another such embodiment, the amount of allopurinol or a pharmaceutically acceptable salt thereof administered to the subject is about 650 mg per day. In another such embodiment, the amount of allopurinol or a pharmaceutically acceptable salt thereof administered to the subject is about 700 mg per day. In another such embodiment, the amount of allopurinol or a pharmaceutically acceptable salt thereof administered to the subject is about 750 mg per day. In another such embodiment, the amount of allopurinol or a pharmaceutically acceptable salt thereof administered to the subject is about 800 mg per day. In another such embodiment, the amount of allopurinol or a pharmaceutically acceptable salt thereof administered to said subject is about 900 mg per day.

In one embodiment of the methods of the present invention, the amount of tranilast or a pharmaceutically acceptable salt thereof administered to a subject is 300 mg per day and the amount of allopurinol or a pharmaceutically acceptable salt thereof administered to the subject is 400 mg, 450 mg, 500 mg, 550 mg, 600 mg, 650 mg, 700 mg, 750 mg, 800 mg, 850 mg or 900 mg per day. In one such embodiment, the amount of allopurinol or a pharmaceutically acceptable salt thereof administered to the subject is 400 mg per day. In another such embodiment, the amount of allopurinol or a pharmaceutically acceptable salt thereof administered to the subject is 500 mg per day. In one such embodiment, he amount of allopurinol or a pharmaceutically acceptable salt thereof administered to the subject is 600 mg per day. In one such embodiment, the amount of allopurinol or a pharmaceutically acceptable salt thereof administered to the subject is 700 mg per day. In one such embodiment, the amount of allopurinol or a pharmaceutically acceptable salt thereof administered to the subject is 800 mg per day. In another such embodiment, the amount of allopurinol or a pharmaceutically acceptable salt thereof administered to said subject is about 900 mg per day.

The pharmaceutical compositions of the present invention have from about 50 mg to about 500 mg of tranilast or a pharmaceutically acceptable salt thereof and from about 150 mg to about 900 mg of allopurinol or a pharmaceutically acceptable salt thereof. For example, the pharmaceutical compositions of the present invention may have about 50 mg, about 100 mg, about 150 mg, about 200 mg, about 250 mg, about 300 mg, about 350 mg, about 400 mg, about 450 mg or about 500 mg of tranilast or a pharmaceutically acceptable salt thereof. The pharmaceutical compositions of the present invention also have about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg or about 900 mg of allopurinol or a pharmaceutically acceptable salt thereof. In one embodiment the pharmaceutical composition includes about 300 mg of tranilast or a pharmaceutically acceptable salt thereof and about 350 mg, about 400 mg, about 450 mg, about 500 mg, about 550 mg, about 600 mg, about 650 mg, about 700 mg, about 700 mg or about 800 mg of allopurinol or a pharmaceutically acceptable salt thereof. In one such embodiment the composition includes about 300 mg of tranilast or a pharmaceutically acceptable salt thereof and about 350 mg of allopurinol or a pharmaceutically acceptable salt thereof. In another such embodiment the composition includes about 300 mg of tranilast or a pharmaceutically acceptable salt thereof and about 400 mg of allopurinol or a pharmaceutically acceptable salt thereof. In another such embodiment the composition includes about 300 mg of tranilast or a pharmaceutically acceptable salt thereof and about 450 mg of allopurinol or a pharmaceutically acceptable salt thereof. In another such embodiment the composition includes about 300 mg of tranilast or a pharmaceutically acceptable salt thereof and about 500 mg of allopurinol or a pharmaceutically acceptable salt thereof. In another such embodiment the composition includes about 300 mg of tranilast or a pharmaceutically acceptable salt thereof and about 550 mg of allopurinol or a pharmaceutically acceptable salt thereof. In another such embodiment the composition includes about 300 mg of tranilast or a pharmaceutically acceptable salt thereof and about 600 mg of allopurinol or a pharmaceutically acceptable salt thereof. In another such embodiment the composition includes about 300 mg of tranilast or a pharmaceutically acceptable salt thereof and about 650 mg of allopurinol or a pharmaceutically acceptable salt thereof. In another such embodiment the composition includes about 300 mg of tranilast or a pharmaceutically acceptable salt thereof. In another such embodiment the composition includes about 300 mg tranilast or a pharmaceutically acceptable salt thereof and about 700 mg of allopurinol or a pharmaceutically acceptable salt thereof. In another such embodiment the composition includes about 300 mg of tranilast or a pharmaceutically acceptable salt thereof and about 750 mg of allopurinol or a pharmaceutically acceptable salt thereof. In another such embodiment the composition includes about 300 mg of tranilast or a pharmaceutically acceptable salt thereof and about 800 mg of allopurinol or a pharmaceutically acceptable salt thereof.

The compositions of the present invention contain tranilast (or a pharmaceutically acceptable salt or solvate thereof) and allopurinol (or a pharmaceutically acceptable salt or solvate thereof). Dosages for use with the present invention (both compositions of the invention and methods of the invention) are provided in Table A below.

TABLE A
Non-limiting dosages¹ of Tranilast and Allopurinol for use with the present invention.

| Tranilast | Tranilast | Tranilast | Tranilast | Tranilast | Tranilast | | Tranilast |
|---|---|---|---|---|---|---|---|
| (mg), | (mg), | (mg), | (mg), | (mg), | (mg), | Tranilast (mg), | (mg), |
| Allopurinol | Allopurinol | Allopurinol | Allopurinol | Allopurinol | Allopurinol | Allopurinol | Allopurinol |
| (mg) | (mg) | (mg) | (mg) | (mg) | (mg) | (mg) | (mg) |
| 25, 100 | 25, 200 | 50, 300 | 50, 400 | 50, 500 | 50, 600 | 50, 700 | 50, 800 |
| 50, 100 | 50, 200 | 100, 300 | 100, 400 | 100, 500 | 100, 600 | 100, 700 | 100, 800 |
| 75, 100 | 100, 200 | 150, 300 | 150, 400 | 150, 500 | 150, 600 | 150, 700 | 150, 800 |
| | 150, 200 | 200, 300 | 200, 400 | 200, 500 | 200 600 | 200, 700 | 200, 800 |
| | 175, 200 | 250, 300 | 250, 400 | 250, 500 | 250, 600 | 250 700 | 250, 800 |
| | | 275, 300 | 300, 400 | 300, 500 | 300, 600 | 300, 700 | 300, 800 |
| 50, 900 | 25, 250 | 50, 350 | 50, 450 | 50, 550 | 50, 650 | 50, 750 | 50, 850 |
| 100, 900 | 50, 250 | 100, 350 | 100, 450 | 100, 550 | 100, 650 | 100, 750 | 100, 850 |
| 150, 900 | 100, 250 | 150, 350 | 150, 450 | 150, 550 | 150, 650 | 150, 750 | 150, 850 |
| 200, 900 | 150, 250 | 200, 350 | 200, 450 | 200, 550 | 200, 650 | 200, 750 | 200, 850 |
| 250, 900 | 200, 250 | 250, 350 | 250, 450 | 250, 550 | 250, 650 | 250, 750 | 250, 850 |
| 300, 900 | 225, 250 | 300, 350 | 300, 450 | 300, 550 | 300, 650 | 300, 750 | 300, 850 |

¹Weights refer to the API portion of the compound.

Tranilast may be included in the pharmaceutical compositions and methods of the present invention in any pharmaceutically acceptable form, including the free acid, esters, co-crystals, and pharmaceutically acceptable salts. In one embodiment tranilast is the free acid is tranilast without any salt or guest in a co-crystal form. In one embodiment, tranilast is in a co-crystal form.

In one embodiment, the condition associated with an elevated serum uric acid level is hyperuricemia, gout, a renal disorder, cardiovascular disease, an aberrant metabolic condition, cognitive impairment, a fatty liver disease or kidney stones.

In other embodiments, where the present invention provides methods for decreasing serum uric acid level in a subject, the subject has hyperuricemia, gout, gout-associated inflammation, a renal disorder, cardiovascular disease, an aberrant metabolic condition, cognitive impairment, fatty liver disease or kidney stones. In one such embodiment, the subject has cognitive impairment. Cognitive impairment may be associated with cerebral vascular conditions, Alzheimer's disease, Parkinson's disease or aging. Schretlen, D. J., et al., Neuropsychology, 2007, Vol. 21, No. 1, 136-140.

In one aspect the condition associated with an elevated serum uric acid level is hyperuricemia. In one such aspect the method comprises reducing inflammation associated with hyperuricemia.

In one aspect the present invention provides methods for treating hyperuricemia in a subject with a condition selected front the group of gout, a renal disorder, cardiovascular disease, an aberrant metabolic condition, cognitive impairment, a fatty liver disease and kidney stones. In one such embodiment, the subject has gout.

In another aspect the condition associated with an elevated serum uric acid level is gout. In one such aspect the method comprises treating gouty symptoms. In another such aspect the method comprises treating gouty attacks. In another such aspect the method comprises reducing the incidence and/or severity of gouty flares. In another aspect the method comprises treating intercritical periods in gout patients. In another such aspect the method comprises preventing, reducing or reversing uric acid crystal formation. In another such embodiment the method comprises reducing uric acid burden. In another such aspect the method comprises reducing the size and/or number of tophi. The size and/or number of tophi may be assessed by, for example, use of CT scans.

In yet another aspect of the present invention the condition associated with an elevated serum uric acid level is a renal disorder. In one such aspect the renal disorder is chronic kidney disease.

In yet another aspect of the present invention the condition associated with an elevated serum uric acid level is kidney stones.

In yet another embodiment of the present invention the condition associated with an elevated serum uric acid level is an increased risk for developing cardiovascular disease.

In yet another aspect of the present invention the condition associated with an elevated serum uric acid level is cardiovascular disease. In one such aspect the cardiovascular disease is coronary artery disease, stroke, peripheral artery disease, congestive heart failure or hypertension. In one such aspect the cardiovascular disease is coronary heart disease. In another such aspect the cardiovascular disease is stroke. In yet another such aspect the cardiovascular disease is peripheral artery disease. In yet another such aspect the cardiovascular disease is congestive heart failure. In yet another such aspect the cardiovascular disease is hypertension.

In yet another aspect of the present invention the condition associated with an elevated serum uric acid level is an aberrant metabolic condition. In one such aspect the aberrant metabolic condition is metabolic syndrome, obesity, hyperlipidemia, hypercholesterolemia, insulin resistance or diabetes. In one such aspect the aberrant metabolic condition is metabolic syndrome. In another such aspect the aberrant metabolic condition is obesity. In yet another such aspect the aberrant metabolic condition is hyperlipidemia. In yet another such aspect the aberrant metabolic condition is insulin resistance. In yet another such aspect the aberrant metabolic condition is diabetes.

In yet another aspect of the present invention the condition associated with an elevated serum uric acid level is cognitive impairment.

In yet another aspect of the present invention the condition associated with an elevated serum uric acid level is a fatty liver disease. In one such embodiment the fatty liver disease is non-alcoholic fatty liver disease (NAFLD). In another such embodiment, the non-alcoholic fatty liver disease is non-alcoholic steatohepatitis (NASH).

In certain embodiments, administration of a pharmaceutical composition of the present invention to a subject in accordance with a method of the present invention decreases a serum uric acid level in the subject by at least about 5%, at least about 10%, at least about 20%, at least about 30%, at least about 40%, at least about 50%, at least about 60%, at least about 70%, at least about 80% or at least about 90%. In certain embodiments, the serum uric acid level in the subject is decreased by at least about 33%. In certain embodiments, the serum uric acid level in the subject is decreased by at least about 50%.

In certain embodiments, administration of a pharmaceutical composition of the present invention to a subject in accordance with a method of the present invention decreases a serum uric acid level in the subject by from about 5% to about 90%, by from about 10% to about 50%, by from about 20% to about 40%, or by from about 25% to about 35%.

In further embodiments, the methods of the present invention comprise administering a pharmaceutical composition of the present invention to a subject whose serum uric acid level is at least about 4.0 mg/dL, at least about 4.5 mg/dL, at least about 5.0 mg/dL, at least about 5.5 mg/dL, at least about 6.0 mg/dL, at least about 6.8 mg/dL, at least about 6.5 mg/dL, at least about 6.8 mg/dL, at least about 7.0 mg/dL, at least about 7.5 mg/dL, at least about 8.0 mg/dL, at least about 8.5 mg/dL, at least about 9.0 mg/dL, at least about 9.5 mg/dL, at least about 10.0 mg/dL, at least about 10.5 mg/dL, or at least about 11.0 mg/dL. In one such embodiment the subject's serum uric acid level is at least 7.0 mg/dL. In another such embodiment the subject's serum uric acid level is at least 7.5 mg/dL. In another such embodiment the subject's serum uric acid level is at least 8.0 mg/dL. In another such embodiment the subject's serum uric acid level is at least 8.5 mg/dL. In another such embodiment the subject's serum uric acid level is at least 9.0 mg/dL. In another such embodiment the subject's serum uric acid level is at least 9.5 mg/dL. In another such embodiment the subject's serum uric acid level is at least 10.0 mg/dL. In another such embodiment the subject's serum uric acid level is at least 10.5 mg/dL. In another such embodiment the subject's serum uric acid level is at least 11.0 mg/dL.

In further embodiments, the methods of the present invention decrease a serum uric acid level in the subject below about 7.0 mg/dL, below about 6.8 mg/dL, below about 6.5 mg/dL, below about 6.0 mg/dL, below about 5.5 mg/dL, below about 5.0 mg/dL, below about 4.5 mg/dL, below about 4.0 mg/dL, below about 3.5 mg/dL, below about 3.0 mg/dL, below about 2.5 mg/dL, below about 2.0 mg/dL or below about 1.5 mg/dL. The appropriate serum uric acid level may vary depending on the subject, and may vary for a given subject over time, depending upon the subject's overall medical condition. Similarly, the appropriate serum uric acid level for one group of subjects sharing a common medical condition may be different from that which is appropriate for a different group of subjects sharing a different medical condition. Thus, it may be advisable to reduce the serum uric acid level of a given group of subjects to, for example, below about 6.0 mg/dL, below about 5.0 mg/dL, and to reduce the serum uric acid level of a different group of subjects to, for example, below about 4.0 mg/dL. In certain embodiments, the methods of the present invention decrease a serum uric acid level in the subject by an amount sufficient to cause the disappearance of tophi over a timeframe of weeks or months. In one embodiment, the methods of the present invention reduce the serum uric acid level of a subject to below about 6.0 mg/dL. In another embodiment, the methods of the present invention reduce the serum uric acid level of a subject to below about 5.0 mg/dL. In another embodiment, the methods of the present invention reduce the serum uric acid level of a subject to below about 4.0 mg/dL.

In some embodiments, a serum uric acid level in the subject is decreased by between about 0.1 to about 10.0 mg/dL. In certain such embodiments a serum uric acid level in the subject is decreased by between about 0.5 to about 8.0 mg/dL, by between about 1.0 to about 6.0 mg/dL, or by between about 2.0 to about 5.0 mg/dL. In certain other embodiment the serum uric acid level in the subject is decreased by between about 1.0 to about 4.0 mg/dL, or by between about 1.0 to about 2.0 mg/dL. Again, the amount of decrease of serum uric acid level that is appropriate may vary depending on the subject, depending upon the subject's overall medical condition. Similarly, the amount of decrease of serum uric acid level that is appropriate for one group of subjects sharing a common medical condition may be different from that which is appropriate for a different group of subjects sharing a different medical condition.

In certain embodiments, the methods of the present invention comprise administering a pharmaceutical composition of the present invention to a subject whose serum uric acid level is within the normal range.

In further embodiments of the methods for treating a condition associated with an elevated serum uric acid level, the subject has gout.

In gout, crystals of monosodium urate (a salt of uric acid) are deposited in joints, e.g., on articular cartilage, as well as in and on tendons and surrounding tissues. These deposits correlate with elevated concentrations of uric acid in the blood stream and are believed to provoke the painful inflammatory reaction that occurs in affected tissues.

Gout is characterized by excruciating, sudden, unexpected, burning pain, as well as by swelling, redness, warmness, and stiffness in the affected joint. Low-grade fever may also be present. The patient usually suffers from two sources of pain. The patient experiences intense pain whenever an affected joint is flexed. The inflammation of the tissues around the joint also causes the skin to be swollen, tender and sore if it is even slightly touched. For example, a blanket or even the lightest sheet draping over the affected area could cause extreme pain. A gout flare is a sudden attack of pain in affected joints, especially in the lower extremities, and most commonly in the big toe.

The frequency of gout flares typically increases over time in a subject who suffers gout. In this fashion, gout progresses from acute gout to chronic gout, which involves repeated episodes of joint pain. Depending on the frequency and severity of the gout flares, chronic gout is also referred to as moderate gout, moderate to severe gout, moderately severe gout, severe gout, and/or refractory gout. Therefore, patients with moderate to severe gout can also be characterized as having chronic gout, and vice versa. In one embodiment, a patient with severe gout has a serum uric acid level greater than or equal to 8.0 mg/dL. In a further embodiment, a patient with severe gout has at least one gout tophus or gouty arthritis or has had at least three gouty flares in an 18 month period.

In acute gout flares, symptoms develop suddenly and usually involve only one or a few joints. The big toe, knee, or ankle joints are most often affected. The pain frequently starts during the night and is often described as throbbing, crushing, or excruciating. The joint appears infected, with signs of warmth, redness, and tenderness. Flares of painful joints may go away in several days, but may return from time to time. Subsequent flares usually last longer.

Acute gout may progress to chronic gout flares, or may resolve without further attacks. The chronic appearance of several attacks of gout yearly can lead to joint deformity and limited joint motion. Nodular uric acid deposits, called tophi, may eventually develop in cartilage tissue, tendons, and soft tissues. These tophi are a hallmark of chronic gout, which usually develop only after a patient has suffered from the disease for many years. Deposits of monosodium urate can also occur in the kidneys of gout sufferers, potentially leading to chronic kidney failure. Chronic gout patients, in one embodiment, include subjects having recurrent or prolonged gout flares, tophus formation, chronic inflammatory arthritis and/or joint destruction associated with gout.

In one embodiment, a patient with moderate to severe gout has 2 or more gout flares in a period of about 6 to about 24 months. In a further embodiment, the patient suffers from gouty arthritis or has at least one gout tophus.

In another embodiment, a patient with moderate to severe gout suffers 2 or more gout flares in a period of about 12 to about 24 months. In a further embodiment, the patient suffers from gouty arthritis or has at least one gout tophus.

In another embodiment, a patient with moderate to severe gout suffers 2 or more gout flares in a period of about 12 to about 18 months. In a further embodiment, the patient suffers from gouty arthritis or has at least one gout tophus.

In yet another embodiment, a patient with moderate to severe gout suffers 2 or more gout flares in a period of about 12 months. In a further embodiment, the patient suffers from gouty arthritis or has at least one gout tophus.

In another embodiment, a patient with moderate to severe gout suffers 3 or more gout flares in a period of about 6 to about 24 months. In a further embodiment, the patient suffers from gouty arthritis or has at least one gout tophus.

In another embodiment, a patient with moderate to severe gout suffers 3 or more gout flares in a period of about 12 to about 24 months. In a further embodiment, the patient suffers from gouty arthritis or has at least one gout tophus.

In another embodiment, a patient with moderate to severe gout suffers 3 or more gout flares in a period of about 12 to about 18 months. In another embodiment, a patient with moderate to severe gout suffers 3 or more gout flares in a period of about 12 months. In a further embodiment, the patient suffers from gouty arthritis or has at least one gout tophus.

In another embodiment, a patient with moderate to severe gout has at least one gout tophus, at least two gout tophi or at least three gout tophi. In yet another embodiment, a patient with moderate to severe gout suffers from gouty arthritis. In a further embodiment, the patient with moderate to severe gout suffers 3 or more gout flares in a period of 12 months.

In some embodiments, the subject has severe gout. In some embodiments, the subject has moderately severe gout. In some embodiments, the subject has chronic gout. In some embodiments, the subject has acute gout. In some embodiments, the subject has refractory gout. In some embodiments, the subject's reduction in serum uric acid level upon administration of allopurinol or a pharmaceutically acceptable salt thereof is less than the median response of subjects administered an equivalent amount of allopurinol or a pharmaceutically acceptable salt thereof. In some embodiments, the subject has had at least one gouty attack. In some embodiments, the subject has uric acid crystal formation determined by aspiration of tophi or by aspiration of synovial fluid of an inflamed joint.

In further embodiments of the methods for treating a condition associated with an elevated serum uric acid level, the subject has a known risk of gouty attack. In some embodiments, the risk of gouty attack is determined by a combination of hyperuricemia and one or more of a history of gouty attack, obesity, diabetes, chronic kidney failure, hypertension, use of diuretic drugs, high purine diet, high fructose diet, exposure to lead, high consumption of red meat and protein, and high alcohol intake.

In some embodiments of the methods for treating gouty symptoms, the gouty symptoms comprise one or more of pain, inflammation, swelling, muscle fatigue, stress feelings, kidney stones, tophi, podagra or myocardial infarction. In one such embodiment the gouty symptoms are tophi.

In some embodiments of the methods for treating uric acid crystal formation, the uric acid crystal formation is in one or more of the joints, under skin, and kidney. In some embodiments, the formations include tophaceous deposits.

In some embodiments of the methods for treating kidney stones, the kidney stones comprise one or more of uric acid, calcium oxalate and calcium phosphate. In some embodiments, the kidney stones are caused by increased uric acid levels and formation of uric acid crystals.

In some embodiments of the methods for treating a renal disorder, the renal disorder is urinary lithiasis, hyperuricemic nephropathy, acute uric acid nephropathy, microalbuminuria, renal dysfunction, impaired glomerular filtration rate, or nephrolithiasis. In some embodiments, the renal disorder is renal insufficiency or chronic kidney disease. In one such embodiment the renal disorder is renal insufficiency. In another such embodiment the renal disorder is chronic kidney disease.

In some embodiments of the methods for treating cardiovascular disease, the cardiovascular disease is hypertension, myocardial infarction, coronary artery disease, cerebrovascular disease, vascular dementia, preeclampsia, heart disease, congestive heart failure, stroke, atherogenesis, thrombogenesis, atherosclerosis, inflammatory disease or peripheral, carotid, or coronary vascular disease. In one such embodiment the cardiovascular disease is hypertension. In another such embodiment the cardiovascular disease is coronary artery disease. In yet another such embodiment the cardiovascular disease is congestive heart failure. In yet another such embodiment the cardiovascular disease is stroke. In yet another such embodiment the cardiovascular disease is atherosclerosis. In yet another such embodiment the cardiovascular disease is peripheral vascular disease.

In some embodiments of the methods for treating an aberrant metabolic condition the aberrant metabolic condition is metabolic syndrome, obesity, hyperlipidemia, insulin resistance or diabetes. In one such embodiment the aberrant metabolic condition is metabolic syndrome. In another such embodiment the aberrant metabolic condition is obesity. In yet another such embodiment the aberrant metabolic condition is hyperlipidemia. In yet another such embodiment the aberrant metabolic condition is insulin resistance. In yet another such embodiment the aberrant metabolic condition is diabetes.

In some embodiments of the methods for treating cognitive impairment, the cognitive impairment is dementia or Alzheimer's disease.

In some embodiments of the methods for treating a fatty liver disease the fatty liver disease is non-alcoholic fatty liver disease (NAFLD). In one such embodiment, the non-alcoholic fatty liver disease is non-alcoholic steatohepatitis (NASH).

In some embodiments, where the present invention provides methods of lowering serum uric acid in a subject that has experienced insufficient lowering of serum uric acid following treatment with a uric acid synthesis inhibitor, uricosuric agent and/or a recombinant uricase, the uric acid synthesis inhibitor is a xanthine oxidase inhibitor. In one such embodiment the xanthine oxidase inhibitor is allopurinol or a pharmaceutically acceptable salt thereof. In another such embodiment the xanthine oxidase inhibitor is febuxostat.

In other embodiments, where the present invention provides methods of lowering serum uric acid in a subject that has experienced insufficient lowering of serum uric acid following treatment with a uric acid synthesis inhibitor and/or uricosuric agent, the uricosuric agent is probenecid. In another such embodiment the uricosuric agent is a uricase.

In one embodiment, where the present invention provides methods of lowering serum uric acid in a subject that has experienced insufficient lowering of serum uric acid following treatment with a xanthine oxidase inhibitor, the xanthine oxidase inhibitor is allopurinol or a pharmaceutically acceptable salt thereof or is febuxostat, or a pharmaceutically acceptable salt thereof.

In any of the methods described herein, the methods cart further comprise measuring serum uric acid levels in the subject before and after administration of a compound of the invention, wherein a decrease in serum uric acid levels after the administration indicates an effective treatment. In a further embodiment, the dosage of the allopurinol and/or tranilast is adjusted if the serum uric acid levels in a subject are not reduced to the desired serum uric acid level, after initial administration of a compound of the invention. In one embodiment, a decrease in serum uric acid levels in a subject to below about 7 mg/dL, or below about 6.8 mg/dL, or below about 6.5 mg/dL, or below about 6.0 mg/dL, or below about 5.5 mg/dL, or below about 5.0 mg/dL, or below about 4.5 mg/dL, or below about 4.0 mg/dL, or below about 3.5 mg/dL, indicates an effective treatment. In one embodiment, if a subject's serum uric acid levels is greater than about 5.5 mg/dL, or greater than about 6.0 mg/dL, or greater than about 6.8 mg/dL or greater than about 7.0 mg/dL after administration of a compound of the invention, the administration is not deemed effective.

Additionally, in any of the methods described herein, dosages of allopurinol and/or tranilast, in one embodiment, are adjusted after initial administration, based on one or more of the following factors: (1) a change in the number gout flares, (2) a change in the number of tophi, (3) a change in the size of one or more tophi, for example the largest tophus, (4) a change in number of inflamed joints due to gouty arthritis, (5) a change in C-reactive protein (CRP) levels. In one embodiment, the above factors are assessed 1 week after dosing, 2 weeks after dosing, 3 weeks after dosing, 4 weeks after dosing, 8 weeks after dosing, 16 weeks after dosing or 24 weeks after dosing. In one embodiment, if the change in one of the above factors is deleterious, e.g., an increase in gout flares or number of tophi, the dose of allopurinol or tranilast is increased, e.g., by 100 mg-700 mg allopurinol, by about 100 mg-600 mg allopurinol, by about 100 mg-500 mg allopurinol, by about 100 mg-400 mg allopurinol, by about 100 mg-300 mg allopurinol, by about 100 mg-200 mg allopurinol, by about 50 mg allopurinol, by 50 mg tranilast, by 50 mg allopurinol and 50 mg tranilast, by 100 mg allopurinol, by 100 mg tranilast, by 100 mg allopurinol and 50 mg tranilast, by 50 mg allopurinol and 100 mg tranilast.

In one embodiment of the methods of the present invention, the subject in need of treatment has normal liver function. In another embodiment, the subject has liver function within two times or three times the upper limit of normal, as measured by liver function tests.

A subject of any of the methods described herein, in one embodiment, has his or her liver function monitored at least once a month for a period of one month after initial treatment, at least once a month for a period of two months after initial treatment, at least once a month for a period of three months after initial treatment, at least once a month for a period of four months after initial treatment, at least once a month for a period of five months after initial treatment, at least once a month for a period of six months after initial treatment or at least once a month for a period of once a month for a year after initial treatment. In one embodiment, monitoring occurs at evenly spaced intervals, e.g., every 30 days, biweekly or weekly.

In another embodiment, a subject of any of the methods described herein has his or her liver function monitored at least twice a month for a period of one month after initial treatment, at least twice a month for a period of two months after initial treatment, at least twice a month for a period of three months after initial treatment, at least twice a month for a period of four months after initial treatment, at least twice a month for a period of five months after initial treatment, at least twice a month for a period of six months after initial treatment or at least twice a month for a period of once a month for a year after initial treatment. In one embodiment, monitoring occurs at evenly spaced intervals, e.g., weekly or biweekly.

The combination therapies disclosed herein can provide a beneficial therapeutic effect, particularly an additive or over-additive effect. In some embodiments the combination therapies disclosed herein can provide an overall reduction of side effects, e.g., adverse effects. In some embodiments the additive or over-additive beneficial therapeutic effect of the combination therapies disclosed herein provides for dose reduction and/or interval extension when compared to the isolated use of the individual therapeutic agents. Also, the effect of Tranilast or a pharmaceutically acceptable salt thereof on reducing the pain associated with inflammation may be of additional benefit during the arthritic flares associated with gout attacks, thus presenting a unique and differentiating therapy for the disease.

### Methods of Decreasing Serum Uric Acid Level in a Subject

In one embodiment, the present invention provides a method of decreasing serum uric acid level in a subject having a condition associated with an elevated serum uric acid level comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof. In a further embodiment, the dosage of the allopurinol and/or tranilast is adjusted if the serum uric acid levels in a subject are not reduced to the desired serum uric acid level, after administration of tranilast and allopurinol. In a further embodiment, a decrease in serum uric acid levels in a subject to below about 7 mg/dL, or below about 6.8 mg/dL, or below about 6.5 mg/dL, or below about 6.0 mg/dL, or below about 5.5 mg/dL, or below about 5.0 mg/dL, or below about 4.5 mg/dL, or below about 4.0 mg/dL, or below about 3.5 mg/dL, indicates an effective treatment. Therefore, in one embodiment, if sUA levels are not reduced to the level indicated, the dose of allopurinol and/or tranilast is increased. In a further embodiment, the dose of allopurinol is increased by about 100 mg-800 mg, by about 100 mg-700 mg, by about 100 mg-600 mg, by about 100 mg-500 mg, by about 100 mg-400 mg, by about 100 mg-300 mg, by about 100 mg-200 mg, by about 50 mg, by about 100 mg, by about 200 mg, by about 300 mg, by about 400 mg, by about 500 mg, by about 600 mg, by about 700 mg, or by about 800 mg.

In one embodiment of the method of decreasing serum uric acid level, the method comprises administering to said subject about 300 mg of tranilast or a pharmaceutically acceptable salt or solvate thereof. In another embodiment of the method, the method comprises administering to said subject about 400 mg, about 500 mg, about 600 mg, about 700 mg, about 800 mg, or about 900 mg allopurinol or a pharmaceutically acceptable salt or solvate thereof.

In another embodiment of the method of decreasing serum uric acid level, the method comprises administering to said subject about 400 mg of allopurinol or a pharmaceutically acceptable salt or solvate thereof. In another embodiment of the method, the method comprises administering to said subject about 500 mg of allopurinol or a pharmaceutically acceptable salt or solvate thereof. In another embodiment of the method, the method comprises administering to said subject about 600 mg of allopurinol or a pharmaceutically acceptable salt or solvate thereof. In another embodiment of the method, the method comprises administering to said subject about 700 mg of allopurinol or a pharmaceutically acceptable salt or solvate thereof. In another embodiment of the method, the method comprises administering to said subject about 800 mg of allopurinol or a pharmaceutically acceptable salt or solvate thereof. In another embodiment of the method, the method comprises administering to said subject about 900 mg of allopurinol or a pharmaceutically acceptable salt or solvate thereof.

In another embodiment of the method of decreasing serum uric acid level, the method reduces said subject's serum uric acid level below 6.0 mg/dL. In another embodiment of the method, the method reduces said subject's serum uric acid level below 5.0 mg/dL. In another embodiment of the method, the method reduces said subject's serum uric acid level below 4.0 mg/dL.

In another embodiment of the method of decreasing serum uric acid level, the method comprises administering to said subject a pharmaceutical composition comprising tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof in said composition is greater than the amount by weight of said tranilast or pharmaceutically acceptable salt or solvate thereof in said composition. In one embodiment of the method, the pharmaceutical composition comprises about 300 mg of tranilast or a pharmaceutically acceptable salt or solvate thereof.

In one embodiment of the method of decreasing serum uric acid level, the pharmaceutical composition comprises about 400 mg, about 500 mg, about 600 mg, about 700 mg, about 800 mg, or 900 mg of allopurinol or a pharmaceutically acceptable salt or solvate thereof. In one embodiment of the method, the pharmaceutical composition comprises about 400 mg of allopurinol or a pharmaceutically acceptable salt or solvate thereof. In one embodiment of the method of decreasing serum uric acid level, the pharmaceutical composition comprises about 500 mg of allopurinol or a pharmaceutically acceptable salt or solvate thereof. In one embodiment of the method of decreasing serum uric acid level, the pharmaceutical composition comprises about 600 mg of allopurinol or a pharmaceutically acceptable salt or solvate thereof. In one embodiment of the method, the pharmaceutical composition comprises about 700 mg of allopurinol or a pharmaceutically acceptable salt or solvate thereof. In one embodiment of the method of decreasing serum uric acid level, the pharmaceutical composition comprises about 800 mg of allopurinol or a pharmaceutically acceptable salt or solvate thereof. In one embodiment of the method, the pharmaceutical composition comprises about 900 mg of allopurinol or a pharmaceutically acceptable salt or solvate thereof.

In one embodiment of the method of decreasing serum uric acid level wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 6.0 mg/dL, below 5.0 mg/dL or below 4.0 mg/dL. In one embodiment of the method of decreasing serum uric acid level wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 5.0 mg/dL. In one embodiment of the method of decreasing serum uric acid level wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 4.0 mg/dL. In one embodiment of the method, allopurinol or a pharmaceutically acceptable salt or solvate thereof is administered in multiple doses.

In one embodiment, the present invention provides a method of decreasing serum uric acid level in a subject comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof.

In one embodiment of the method, the method of decreasing serum uric acid level comprises administering to said subject about 300 mg of tranilast or a pharmaceutically acceptable salt or solvate thereof. In another embodiment of the method, the method comprises administering to said subject about 400 mg, about 500 mg, about 600 mg, about 700 mg, about 800 mg, or 900 mg of allopurinol or a pharmaceutically acceptable salt or solvate thereof. In another embodiment of the method, the method comprises administering to said subject about 400 mg of allopurinol or a pharmaceutically acceptable salt or solvate thereof. In another embodiment of the method, the method comprises administering to said subject about 500 mg of allopurinol or a pharmaceutically acceptable salt or solvate thereof. In another embodiment of the method, the method comprises administering to said subject about 600 mg of allopurinol or a pharmaceutically acceptable salt or solvate thereof. In another embodiment of the method, the method comprises administering to said subject about 700 mg of allopurinol or a pharmaceutically acceptable salt or solvate thereof. In another embodiment of the method, the method comprises administering to said subject about 800 mg of allopurinol or a pharmaceutically acceptable salt or solvate thereof. In another embodiment of the method, the method comprises administering to said subject about 900 mg of allopurinol or a pharmaceutically acceptable salt or solvate thereof. In another embodiment of the method, the method reduces said subject's serum uric acid level below 6.0 mg/dL. In another embodiment of the method, the method reduces said subject's serum uric acid level below 5.0 mg/dL. In another embodiment of the method, the method reduces said subject's serum uric acid level below 4.0 mg/dL.

In one embodiment of the method, the method of decreasing serum uric acid level comprises administering to said subject a pharmaceutical composition comprising tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof in said composition is greater than the amount by weight of said tranilast or pharmaceutically acceptable salt or solvate thereof in said composition.

In one embodiment of the method, the pharmaceutical composition comprises about 300 mg of tranilast or a pharmaceutically acceptable salt or solvate thereof. In one embodiment of the method, the pharmaceutical composition comprises about 400 mg, about 500 mg, about 600 mg, about 700 mg, about 800 mg, or 900 mg of allopurinol or a pharmaceutically acceptable salt or solvate thereof.

In one embodiment of the method, the pharmaceutical composition comprises about 400 mg of allopurinol or a pharmaceutically acceptable salt or solvate thereof. In one embodiment of the method, the pharmaceutical composition comprises about 500 mg of allopurinol or a pharmaceutically acceptable salt or solvate thereof. In one embodiment of the method, the pharmaceutical composition comprises about 600 mg of allopurinol or a pharmaceutically acceptable salt or solvate thereof. In one embodiment of the method, the pharmaceutical composition comprises about 700 mg of allopurinol or a pharmaceutically acceptable salt or solvate thereof. In one embodiment of the method, the pharmaceutical composition comprises about 800 mg of allopurinol or a pharmaceutically acceptable salt or solvate thereof. In one embodiment of the method, the pharmaceutical composition comprises about 900 mg of allopurinol or a pharmaceutically acceptable salt or solvate thereof.

in one embodiment of the method of decreasing serum uric acid level wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 6.0 mg/dL. In one embodiment of the method wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 5.0 mg/dL. In one embodiment of the method wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 4.0 mg/dL.

### Methods for Treating Hyperuricemia

In one embodiment, the present invention provides a method of treating hyperuricemia in a subject comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof. In a further embodiment, the dosage of the allopurinol and/or tranilast is adjusted if the serum uric acid levels in a subject are not reduced to the desired serum uric acid level, after administration of tranilast and allopurinol. In a further embodiment, a decrease in serum uric acid levels in a subject to below about 7 mg/dL, or below about 6.8 mg/dL, or below about 6.5 mg/dL, or below about 6.0 mg/dL, or below about 5.5 mg/dL, or below about 5.0 mg/dL, or below about 4.5 mg/dL, or below about 4.0 mg/dL, or below about 3.5 mg/dL indicates an effective treatment. Therefore, in one embodiment, if sUA levels are not reduced to the level indicated, the dose of allopurinol and/or tranilast is increased. In a further embodiment, the dose of allopurinol is increased by about 100 mg-700 mg, by about 100 mg-600 mg, by about 100 mg-500 mg, by about 100 mg-400 mg, by about 100 mg-300 mg, by about 100 mg-200 mg, by about 50 mg, by about 100 mg, by about 200 mg, by about 300 mg, by about 400 mg, by about 500 mg, by about 600 mg, by about 700 mg, or by about 800 mg.

In one embodiment of the method the method comprises administering to said subject about 300 mg of tranilast or a pharmaceutically acceptable salt or solvate thereof. In another embodiment the method, the method comprises administering to said subject about 400 mg, about 500 mg, about 600 mg, about 700 mg, about 800 mg, or about 900 mg of allopurinol or a pharmaceutically acceptable salt or solvate thereof. In another embodiment of the method, the method comprises administering to said subject about 400 mg of allopurinol or a pharmaceutically acceptable salt or solvate thereof. In another embodiment of the method, the method comprises administering to said subject about 500 mg of allopurinol or a pharmaceutically acceptable salt or solvate thereof. In another embodiment of the method, the method comprises administering to said subject about 600 mg of allopurinol or a pharmaceutically acceptable salt or solvate thereof. In another embodiment of the method, the method comprises administering to said subject about 700 mg of allopurinol or a pharmaceutically acceptable salt or solvate thereof. In another embodiment of the method, the method comprises administering to said subject about 800 mg of allopurinol or a pharmaceutically acceptable salt or solvate thereof. In another embodiment of the method, the method comprises administering to said subject about 900 mg of allopurinol or a pharmaceutically acceptable salt or solvate thereof.

In another embodiment of the method for treating hyperuricemia, the method reduces said subject's serum uric acid level below 6.0 mg/dL. In another embodiment of the method for treating hyperuricemia, the method reduces said subject's serum uric acid level below 5.0 mg/dL. In another embodiment of the method, the method reduces said subject's serum uric acid level below 4.0 mg/dL. In one embodiment of the method for treating hyperuricemia, the comprises administering to said subject a pharmaceutical composition comprising tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof in said composition is greater than the amount by weight of said tranilast or pharmaceutically acceptable salt or solvate thereof in said composition.

In one embodiment of the method for treating hyperuricemia, the pharmaceutical composition comprises about 300 mg of tranilast or a pharmaceutically acceptable salt or solvate thereof. In one embodiment of the method, the pharmaceutical composition comprises about 400 mg, about 500 mg, about 600 mg, about 700 mg, about 800 mg, or about 900 mg of allopurinol or a pharmaceutically acceptable salt or solvate thereof. In one embodiment of the method for treating hyperuricemia, the pharmaceutical composition comprises about 400 mg of allopurinol or a pharmaceutically acceptable salt or solvate thereof.

In one embodiment of the method for treating hyperuricemia, the pharmaceutical composition comprises about 500 mg of allopurinol or a pharmaceutically acceptable salt or solvate thereof. In one embodiment of the method, the pharmaceutical composition comprises about 600 mg of allopurinol or a pharmaceutically acceptable salt or solvate thereof. In one embodiment of the method for treating hyperuricemia, the pharmaceutical composition comprises about 700 mg of allopurinol or a pharmaceutically acceptable salt or solvate thereof. In one embodiment of the method, the pharmaceutical composition comprises about 800 mg of allopurinol or a pharmaceutically acceptable salt or solvate thereof.

In one embodiment of the method for treating hyperuricemia, the pharmaceutical composition comprises about 900 mg of allopurinol or a pharmaceutically acceptable salt or solvate thereof. In one embodiment of the method wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 6.0 mg/dL. In one embodiment of the method for treating hyperuricemia wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 50 mg/dL. In one embodiment of the method for treating hyperuricemia wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 4.0 mg/dL.

### Methods for Treating Gout

In one embodiment, the present invention provides a method of treating gout in a subject comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof. In a further embodiment, the dosage of the allopurinol and/or tranilast is adjusted if the serum uric acid levels in a subject are not reduced to the desired serum uric acid level, after administration of tranilast and allopurinol. In a further embodiment, a decrease in serum uric acid levels in a subject to below about 7 mg/dL, or below about 6.8 mg/dL, or below about 6.5 mg/dL, or below about 6.0 mg/dL, or below about 5.5 mg/dL, or below about 5.0 mg/dL, or below about 4.5 mg/dL, or below about 4.0 mg/dL, or below about 3.5 mg/dL indicates an effective treatment. Therefore, in one embodiment, if sUA levels are not reduced to the level indicated, the dose of allopurinol and/or tranilast is increased. In a further embodiment, the dose of allopurinol is increased by about 100 mg-700 mg, by about 100 mg-600 mg, by about 100 mg-500 mg, by about 100 mg-400 mg, by about 100 mg-300 mg, by about 100 mg-200 mg, by about 50 mg, by about 100 mg, by about 200 mg, by about 300 mg, by about 400 mg, by about 500 mg, by about 600 mg, by about 700 mg, or by about 800 mg.

In one embodiment of the method of treating gout, the method comprises administering to said subject about 300 mg of tranilast or a pharmaceutically acceptable salt or solvate thereof. In another embodiment of the method, the method comprises administering to said subject about 400 mg, about 500 mg, about 600 mg, about 700 mg, about 800 mg, or about 900 mg of allopurinol or a pharmaceutically acceptable salt or solvate thereof.

In another embodiment of the method of treating gout, the method comprises administering to said subject about 400 mg of allopurinol or a pharmaceutically acceptable salt or solvate thereof. In another embodiment of the method of treating gout, the method comprises administering to said subject about 500 mg of allopurinol or a pharmaceutically acceptable salt or solvate thereof. In another embodiment of the method, the method comprises administering to said subject about 600 mg of allopurinol or a pharmaceutically acceptable salt or solvate thereof. In another embodiment of the method of treating gout, the method comprises administering to said subject about 700 mg of allopurinol or a pharmaceutically acceptable salt or solvate thereof. In another embodiment of the method of treating gout, the method comprises administering to said subject about 800 mg of allopurinol or a pharmaceutically acceptable salt or solvate thereof. In another embodiment of the method of treating gout, the method comprises administering to said subject about 900 mg of allopurinol or a pharmaceutically acceptable salt or solvate thereof.

In another embodiment of the method of treating gout, the method reduces said subject's serum uric acid level below 6.0 mg/dL. In another embodiment of the method of treating gout, the method reduces said subject's serum uric acid level below 5.0 mg/dL. In another embodiment of the method of treating gout, the method reduces said subject's serum uric acid level below 4.0 mg/dL.

In one embodiment of the method of treating gout, the method comprises administering to said subject a pharmaceutical composition comprising tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof in said composition is greater than the amount by weight of said tranilast or pharmaceutically acceptable salt or solvate thereof in said composition. In one embodiment of the method of treating gout, the pharmaceutical composition comprises about 300 mg of tranilast or a pharmaceutically acceptable salt or solvate thereof. In one embodiment of the method of treating gout of treating gout, the pharmaceutical composition comprises about 400 mg, about 500 mg, about 600 mg, about 700 mg, about 800 mg, or about 900 mg of allopurinol or a pharmaceutically acceptable salt or solvate thereof. In one embodiment of the method of treating gout, the pharmaceutical composition comprises about 400 mg of allopurinol or a pharmaceutically acceptable salt or solvate thereof. In one embodiment of the method of treating gout, the pharmaceutical composition comprises about 500 mg of allopurinol or a pharmaceutically acceptable salt or solvate thereof. In one embodiment of the method of treating gout, the pharmaceutical composition comprises about 600 mg of allopurinol or a pharmaceutically acceptable salt or solvate thereof. In one embodiment of the method of treating gout, the pharmaceutical composition comprises about 700 mg of allopurinol or a pharmaceutically acceptable salt or solvate thereof. In one embodiment of the method, the pharmaceutical composition comprises about 800 mg of allopurinol or a pharmaceutically acceptable salt or solvate thereof. In one embodiment of the method of treating gout, the pharmaceutical composition comprises about 900 mg of allopurinol or a pharmaceutically acceptable salt or solvate thereof.

In one embodiment of the method of treating gout wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 6.0 mg/dL. In one embodiment of the method wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 5.0 mg/dL. In one embodiment of the method wherein a pharmaceutical composition comprising tranilast or a pharmaceutically acceptable salt or solvate thereof and allopurinol or a pharmaceutically acceptable salt or solvate thereof is administered, the method reduces said subject's serum uric acid level below 4.0 mg/dL.

### I. Hyperuricemia

The present invention provides compositions and methods for treating hyperuricemia and related disorders. Hyperuricemia may be defined as a serum urate concentration greater than or equal to 6.8 mg/dL. At serum urate levels greater than or equal to 6.8 mg/dL, uric acid crystals can precipitate out of solution and deposit in joints and other body tissues where they can produce an inflammatory response, neutrophil recruitment, and the production of proinflammatory cytokines as well as other inflammatory mediators. In extremities where body temperatures may be lower than core body temperatures, uric acid crystals may precipitate at lower concentrations, such as 6.0 mg/dL, or lower. Hyperuricemia may be due to overproduction of uric acid. For example, overproduction of uric acid occurs in a variety of metabolic derangements or medical disorders. Alternatively, hyperuricemia may be result of underexcretion of uric acid, such as conditions due to alterations in renal function. Hyperuricemia can lead to hyperuricosuria, which refers to excessive amounts of uric acid in the urine.

Numerous causes of hyperuricemia have been identified. Primary causes are innate to a subject and include genetic disorders such as hypoxanthine phosphoribosyltransferase deficiency and increased phosphoribosyl pyrophosphatase activity. Secondary causes are acquired disorders. These include hereditary fructose intolerance, glycogen storage disease, myeloproliferative disease, lymphoproliferative disease, hemolytic anemia, psoriasis, obesity, renal insufficiency, lead intoxication, chronic beryllium disease, sarcoidosis, and various drugs, e.g., low-dose salicylates, diuretics, pyrazinamide, ethambutol, nicotinamide and ethanol. Table 1 list various causes in terms of their pathophysiology.

TABLE 1
Causes of Hyperuricemia
Urate Overproduction

| | | |
|---|---|---|
| Primary idopathic | Myeloproliferative diseases | Rhabdomyolysis |
| HPRT deficiency | Polycythemia vera | Exercise |
| PRPP synthetase | Psoriasis | Alcohol |
| overactivity | | |
| Hemolytic processes | Paget's disease | Obesity |
| Lymphoproliferative | Glycogenosis III, V and VII | Purine-rich diet |
| diseases | | |

Decreased Uric Acid Excretion

| | | |
|---|---|---|
| Primary idiopathic | Starvation ketosis | Drug ingestion |
| Renal insufficiency | Berylliosis | Salicylates |
| | | (>2 g/d) |
| Polycystic kidney disease | Sarcoidosis | Diuretics |
| Diabetes insipidus | Lead intoxication | Alcohol |
| Hypertension | Hypothyroidism | Levodopa |
| Acidosis | Hyperparathyroidism | Ethambutol |
| Lactic Acidosis | Toxemia of pregnancy | Pyrazinamide |
| Diabetic ketoacidosis | Bartter's syndrome | Nicotinic acid |
| | Down syndrome | Cyclosporine |

Combined Mechanisms

| | | |
|---|---|---|
| Glucose-6-phosphatase | Fructose-1-phosphate | Alcohol |
| deficiency | aldolase deficiency | Shock |

Certain events can cause hyperuricemia. Rapid purine degradation can cause hyperuricemia, e.g., in conditions of rapid cell proliferation or death, e.g., leukimic blast crises, cytotoxic cancer treatment, hemolysis or rhabdomylosis. Hyperuricemia can also result from excessive degradation of ATP from muscles, e.g., after exercise or due to glycogen storage diseases III, V and VII. Relatedly, hyperuricemia can be caused by myocardial infarction, smoke inhalation, and acute respiratory failure.

The methods and compositions of the present invention can be used to treat hyperuricemia related to most, if not all, of the above causes, e.g., by reducing serum uric acid levels.

(a) Disorders Associated with Hyperuricemia

Disorders associated with high levels of serum uric acid levels include, but are not limited to hyperuricemia, gout, urinary lithiasis, hyperuricemic nephropathy, acute uric acid nephropathy, cardiovascular disorders, renal disorders, metabolic disorders, fatty liver diseases, kidney stones and the like. Complications resulting from high levels of uric acid and uric acid crystal formation include, but are not limited to, muscle spasm, localized swelling, inflammation, joint pains, muscle fatigue, stress feelings, and myocardial infarction. The present invention provides compositions and methods for treating hyperuricemia and such related disorders.

Gout is a group of metabolic rheumatic disorders caused by aberrant purine metabolism and hyperuricemia and is the most common cause of an inflammatory arthropathy in middle-aged men. Gout is essentially a disorder of urate metabolism. Deposition of urate crystals in hyperuricemic individuals results in acute gout, characterized by agonizing pain and inflammation of rapid onset, most frequently affecting the first metatarsophalangeal joint. It can take decades for uric acid levels to rise to levels where uric acid crystals precipitate. Such precipitation can activate the NLRP3 (NALP3) inflammasome and result in a gouty attack. Hyperuricemia is associated with an increased risk of developing gout, and the risk of gout increases with the degree and duration of the hyperuricemia. Hyperuricemia in gout is typically accompanied by renal complications and suboptimal excretion of uric acid. Gouty attacks are typically severely painful and disabling.

A variety of risk factors have been identified for gout. In addition to hyperuricemia, these include obesity, diabetes, chronic kidney failure, hypertension, use of diuretic drugs, high purine diet, high fructose diet, exposure to lead, consumption of red meat and protein, and alcohol intake. See also Table 1. Gouty attack can be precipitated by perioperative ketosis in surgical patients, reduced body temperature, e.g., while sleeping, and by dehydration, e.g., by use of diuretic drugs. Genetic risk factors for gout and hyperuricemia have also been identified. Genetic deficiencies that can lead to increased serum urate levels include deficiency of the enzyme hypoxanthine-guaninephosphoribosyl transferase (HPRT), and overactivity of PPriboseP synthase (also known as PRPP (phosphoribosylpyrophosphate) synthetase). Inherited diseases including familial juvenile hyperuricemic nephropathy (FJHN) and autosomal-dominant medullary cystic kidney disease (ADMCKD) can also lead to reduced excretion of uric acid. Lesch-Nyhan syndrome (LNS), also known as Nyhan's syndrome or Kelley-Seegmiller syndrome, is a rare, inherited disorder caused by a deficiency of the enzyme hypoxanthine-guanine phosphoribosyltransferase (HGPRT), produced by mutations in the HPRT gene. LNS can lead to hyperuricemia, hyperuricosuria and corresponding gout and kidney problems. Polymorphisms in a variety of urate transporters have been identified as risk factors for hyperuricemia and gout. These urate transporters include SLC22A12 (URAT1), solute carrier family 2 (facilitative glucose transporter), member 9 gene (SLC2A9; Glut9), ABCG2 and SLC17A3. See, e.g., Cameron J S and Simmonds H A, Hereditary hyperuricemia and renal disease. Semin Nephrol, 2005 25:9-18; Bleyer A J and Hart T C, Genetic factors associated with gout and hyperuricemia. Adv Chronic Kidney Dis. 2006 13:124-30; Enomoto A., et al. Molecular identification of a renal urate anion exchanger that regulates blood urate levels. Nature 2002 417:447-52; Vitart V., et al. SLC2A9 is a newly identified urate transporter influencing serum urate concentration, urate excretion and gout. Nat Genet 2008 40:437-42; Dehghan A., et al. Association of three genetic loci with uric acid concentration and risk of gout: a genome-wide association study. Lancet 2008 372:1953-61.

Gout can be either acute or chronic. Triggers for acute gouty attacks include infection, intravenous contrast media, acidosis, and rapid fluctuations in serum uric acid concentrations such as with trauma, surgery, psoriasis flare-ups, initiation of chemotherapy, diuretic therapy, and stopping or starting allopurinol or a pharmaceutically acceptable salt thereof. Acute attacks usually begin in the joints of lower extremities. The attacks are characterized by joint pain and swelling. The first attack often comprises podagra, a sudden, unexplained swelling and pain of the big toe joint on just one foot. During an attack, which can last several days, pain can be so severe that patients are often unable to wear clothing or even touch bedsheets. Recurrent acute attacks can lead to chronic tophaceous deposits. Tophi are crystallized uric acid deposits that form firm swellings in joints, cartilage and bone. Tophi deposits sometimes disrupt the skin, exposing large chalky nodules. Extensive tophi can erode bone or other tissues and may require surgical removal.

In some patients, current medical treatments are ineffective at controlling serum uric acid levels at less than 6.0 mg/dL. Patients with such recalcitrant disease are deemed to have “refractory gout” and my exhibit severe clinical manifestations, including recurrent gout attacks, persistent swollen and painful joints, chronic pain, and progressive tophaceous deposits and joint damage. Refractory gout can have various causes, including but not limited to ineffectiveness of, or intolerance to, current treatments. Allopurinol or a pharmaceutically acceptable salt thereof remains the most frequent therapeutic for such patients, but it is only effective in some cases and additional therapies are often required.

Patients having “severe gout” include those with serum uric acid levels that are greater than or equal to 8.0 mg/dL and have at least one gout tophus or gouty arthritis or have had at least three gouty flares in the past 18 months. Patients having “moderately severe gout” include those with serum uric acid levels that are greater than or equal to 8.0 mg/dL and experiencing two or more flares within a 12 to 18 month period. Refractory gout comprises patients with severe gout wherein, in addition, conventional therapies are contraindicated or have been or become ineffective. For example, the patient may have a history of hypersensitivity or of failure to normalize serum uric acid (sUA) with at least 3 months of treatment with allopurinol or a pharmaceutically acceptable salt thereof at the maximum labeled dose (800 mg/dL in the U.S.), or at medically appropriate lower dose based on dose-limiting toxicity or dose-limiting co-morbidity.

Medical complications arising from increased levels of serum uric acid are not limited to gout. High serum uric acid levels, even in the absence of uric acid crystal deposits, have been linked to a wide variety of cardiovascular and other conditions, including hypertension, metabolic syndrome, hyperlipidemia, insulin resistance, coronary artery disease, cerebrovascular disease, vascular dementia, preeclampsia, heart disease and kidney disease. High levels of uric acid can predict the onset of hypertension, obesity, diabetes and kidney disease. Risk factors that may contribute to hyperuricemia-related hypertension include those for risk of gout, e.g., dietary, environmental and genetic factors, as described herein. Such hypertension may further be related to chronic kidney disease and other renal diseases, e.g., microalbuminuria and renal dysfunction in subjects with normal renal function and impaired glomerular filtration rate in type 1 diabetics without proteinuria. Other renal problems associated with hyperuricemia include nephrolithiasis, urate nephropathy, and uric acid nephropathy. Nephrolithiasis, or kidney stones, are found most often in gout patients comprising uric acid stones, although hyperuricemia is also associated with other types of stones, e.g., calcium oxalate or calcium phosphate stones in non-gouty patients. Without being bound by theory, uric acid may act to seed calcium deposits. Urate nephropathy manifests from severe gout and is characterized by urate crystals in the renal interstitium. Uric acid nephropathy can cause renal failure from deposition of large amounts of crystals in the renal collecting ducts, pelvis and ureters. Other cardiovascular diseases associated with hyperuricemia include peripheral, carotid, and coronary vascular disease, stroke, preeclampsia, and vascular dementia. In some cases, drugs used to treat hyperuricemia, e.g., allopurinol or a pharmaceutically acceptable salt thereof, may be effective in treating diabetes, hypertension, and other uric acid related disorders. See, e.g., Feng et al., Uric Acid and Cardiovascular Risk, N Engl J Med 2008 359:1811-21.

Gout patients have higher death rates from all causes, although gout associated mortality is largely related to cardiovascular complications. In some cases, such cardiovascular complications relate to high serum uric acid levels, as described above. For example, hypertension is often observed in subjects with hyperuricemia. Higher serum uric acid levels are also associated with ongoing inflammatory response. For example, hyperuricemic patients often display higher levels of serum markers of inflammation, e.g., C reactive protein, fibrinogen, interleukin-6 (IL-6), and increased neutrophil count. The serum of gout patients contains high levels of inflammatory markers even in the absence of an ongoing gouty attack. And during attack, crystal formation activates monocytes and stimulates the release of inflammatory markers including tumor necrosis factor-α, IL-1, IL-6, IL-8, and cyclooxygenase-2. Ongoing low-grade inflammation among patients with gout may promote atherogenesis and thrombogenesis. Similar complications are also observed in other inflammatory rheumatic disorders associated with higher risk of cardiovascular disease, e.g., rheumatoid arthritis or systemic lupus erythematosus.

The present invention provides compositions and methods for treating hyperuricemia and such related disorders as described herein, including, but not limited to, gout, severe gout, refractory gout, chronic gout, cardiovascular disorders and related disorders, renal disorders and related disorders, fatty liver disease, kidney stones and aberrant metabolic conditions.

### (b) Diagnosis

One standard for diagnosis of gout comprises aspiration of tophi or synovial fluid from an inflamed joint. Tophi are crystallized uric acid deposits that form firm swellings in joints, cartilage and bone. Synovial fluid is a thick fluid which lubricates and cushions synovial joints, e.g., the wrist, elbow, knee, shoulder and hip joints. Needlelike monosodium urate crystals observed in the synovial fluid when viewed under a microscope are highly indicative of gout. However, aspiration techniques are not performed routinely in the clinical setting for various reasons, e.g., lack of availability of synovial fluid, time of the procedure and lack of physician experience. Alternately, clinical indications of disease, e.g., podagra, swollen and painful joints, tophi, and elevated serum urate can indicate hyperuricemia and gout. In some approaches, serum urate levels are measured during and following a gouty attack. Sometimes, elevated levels are only observed post-attack, e.g., two weeks later. In patients without hyperuricemia two weeks post-attack, e.g., uric acid levels below 4.0 mg/dL, gout is typically considered unlikely. Radiology can also assist in diagnosis of gout, e.g., to ascertain joint damage and urate deposits. Radiological techniques include x-ray film, computed tomography (CT) scans, magnetic resonance imaging (MRI), Dual Energy Computed Tomography (DECT), and ultrasound. See, e.g., Schlesinger, Diagnosis of Gout: Clinical, Laboratory, and Radiologic Findings, Am J Manag Care. 2005 11:S443-S450; Dore, The Gout Diagnosis, Cleve Clin J Med. 2008 75:S17-S21. The aforementioned diagnostic techniques also may be used to monitor the efficacy of treatment.

Diagnosis of other conditions associated with an elevated serum uric acid level, for example, hyperuricemia, gout-associated inflammation, renal disorders, cardiovascular disease, aberrant metabolic conditions, fatty liver disease or kidney stones, may be performed according to current medical standards.

The methods of the present invention may be used to treat, prevent and/or ameliorate the conditions and/or diseases described herein. Consequently, as used herein the term “treating” includes treating, preventing and/or ameliorating the condition or disease to which it refers. In certain embodiment the methods of the present invention treat the conditions and/or diseases described herein. In other embodiments the methods of the present invention prevent the conditions and/or diseases described herein. In other embodiments the methods of the present invention ameliorate the conditions and/or diseases described herein.

As used herein the term “elevated serum uric acid level” means serum uric acid level greater than normal. In some instances, elevated serum uric acid levels are above the mean level in a given population, such as gender and/or age.

As used herein, the term “weight ratio” refers to the ratio of the weight of the respective non-salt active pharmaceuticals ingredients (API). Thus, for example, a “weight ratio” of 2:1 of allopurinol or pharmaceutically acceptable salt thereof to tranilast or pharmaceutically acceptable salt thereof means that the amount by weight of allopurinol API is two times the amount by weight of tranilast API. In the case of pharmaceutically acceptable salts of allopurinol and/or tranilast (e.g., sodium salts) it does not take into account the weight of the counter ion (e.g., sodium). In the case of co-crystals of tranilast, it does not take into account the weight of the guest molecule. In the case of esters of tranilast, the weight ratio does not take into account the weight of the ester moiety.

As used herein the term “hyperuricemia” means serum uric acid level greater than the normal level for the population. In some instances, hyperuricemia includes serum uric acid levels greater than or equal to 6.0 mg/dL, 6.8 mg/dL, 7 mg/dL or 8 mg/dL.

As used herein the term “severe gout” includes gout present in a subject having serum uric acid levels that are greater than or equal to 8.0 mg/dL and have at least one gout tophus or gouty arthritis or have had at least three gouty flares in the past 18 months.

As used herein the term “chronic gout” includes gout present in a subject having recurrent or prolonged gout flares, tophus formation, chronic inflammatory arthritis and/or joint destruction associated with gout.

As used herein the term “acute gout” includes gout present in a subject that has had or is having at least one gouty symptom, such as a gout flare or gouty attack.

As used herein the term “gout-associated inflammation” refers to local or systemic inflammation due to immune response to urate crystals.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

### Hyperuricemia and Gout

Hyperuricemia and chronic gout are treated with agents that lower urate levels, e.g., xanthine oxidase inhibitors and uricosuric agents, thereby reducing uric acid levels and potential crystal formation. Xanthine oxidase is involved in purine metabolism and inhibiting the enzyme reduces uric acid levels. Allopurinol or a pharmaceutically acceptable salt thereof, a xanthine oxidase inhibitor, is the current first line standard of care for lowering urate levels. Another xanthine oxidase inhibitor, Febuxostat, was approved for treatment of gout in February 2009. Other xanthine oxidase inhibitors include oxypurinol, tisopurine, inositols (e.g., phytic acid and myoinositol) and potentially propolis. Uricosuric agents enhance excretion of uric acid and generally act by lowering the absorption of uric acid from the kidneys back to the blood, e.g., by inhibiting urate transporters, e.g, SLC22A12. Probenecid is the most commonly used uricosuric agent in the U.S. and may be given in combination with allopurinol or a pharmaceutically acceptable salt thereof to refractory gout patients. Benzbromarone and sulfinpyrazone are also used as first line uricosuric agents. Guaifenesin, losartan, atorvastatin, amlodipine, adrenocorticotropic hormone (ACTH or corticotropin), fenofibrate and cortisone also have uricosuric effects. Additionally, other uricosuric agents are being developed or are in clinical trials, such uricase enzymes including rasburicase or the pegylated uricase enzyme PURICASE® (Pegloticase), which has completed Phase III trials. Uricase or urate oxidase enzymes can lower uric acid levels by converting uric acid into allantoin, a benign end metabolite which is easily excreted in the urine. IL-6 has also been shown to reduce serum uric acid levels and proposed as a treatment for hyperuricemia and gout. See U.S. Pat. No. 6,007,804, issued Dec. 28, 1999 and entitled “IL-6 as serum uric acid decreasing compound.” Interleukin 1 (IL-1) antagonists are being developed for chronic gout. For example, Canakinumab (ACZ885) is a human monoclonal antibody targeted at IL-1 beta, being developed by Novartis for the treatment of rheumatoid arthritis and gout. Rilonacept, marketed under the trade name Arcalyst by Regeneron Pharmaceuticals, is under going trials is a dimeric fusion protein and IL-1 blocker, also undergoing trials as a treatment for gout. See also U.S. Patent Publication No. 2008/0300185, filed Oct. 19, 2007 and entitled “Use of IL-1 antagonists to treat gout and pseudogout,” Diet and lifestyle can be modified to reduce urate levels, e.g., lowering red meat or alcohol consumption, or substituting alternate treatments for diuretic drugs.

Acute gout typically presents with inflammation, pain and swelling. Urate-lowering therapies, described above for hyperuricemia and chronic gout, are usually not used until the acute phase of gout has resolved because fluctuations in serum uric acid can exacerbate the inflammatory process. Therapy is generally directed at reducing the inflammation, pain and swelling, e.g., anti-inflammatory agents and pain killers. Nonsteroidal anti-inflammatory drugs (NSAIDs) or corticosteroids are typically given for acute gouty attacks, depending on co-morbidities. NSAIDS include, but are not limited to, indomethacin, naproxen and sulindac. Corticosteroids include, but are not limited to, prednisone or methylprednisone. Colchicine is given as a second line therapy, but has toxicities at higher levels. For example, Colchicine can lead to bone marrow suppression and neuromyopathy in patients with severe renal or hepatic impairment. More common adverse effects include nausea, vomiting, and diarrhea. In some cases, opioid agents are given to acute gout sufferers.

The pharmaceutical compositions of the invention can be used in combination with one or more agents described herein for reducing uric acid or otherwise treating gout. When a combination use is desired, the composition of the invention and the one or more gout treatments can be administered or applied sequentially or simultaneously.

In some embodiments of the invention, methods and compositions of the invention are administered before, concurrent to or after treatment of a subject in need thereof with one or more standards of care used to treat gout. Examples of Standards of Care for gout include but are not limited to administration of one or more therapeutic agents to treat pain and reduce urate in blood. For example, drugs used to lower the amount of urate or analgesic drugs may be administered before, concurrent to or after treatment of a subject in need thereof with pharmaceutical compositions and methods of the present invention.

In some embodiments, pharmacologically active compounds, e.g., for the treatment inflammation, pain, or hyperuricemia, are administered before, concurrent to or after treatment of a subject in need thereof with pharmaceutical compositions and methods of the present invention.

Uricase enzymes. In one embodiment, a pharmaceutical composition of the invention is administered before, concurrently or subsequent to administration of a uricase enzyme. Uricase or urate oxidase enzymes are found in many mammals but not humans. They can lower uric acid levels by converting uric acid into allantoin, a benign end metabolite which is easily excreted in the urine. Uricase enzymes include, but are not limited to, rasburicase or a pegylated uricase enzyme (PEG-uricase). In some embodiments, the pegylated uricase enzyme is PURICASE® (Pegloticase).

Non-steroidal anti-inflammatory drugs (NSAIDs). In one embodiment, a compound of the invention is administered before, concurrently or subsequent to administration of one or more non-steroidal anti-inflammatory drugs (NSAIDs). For example, administration of an NSAID can reduce the pain and inflammation experienced with gout. A non-limiting list of NSAIDs includes diclofenac, indomethacin, naproxen, sulindac and lumiracoxib. Further NSAIDs capable of use with methods and compositions of the invention are disclosed in U.S. Pat. Nos. 7,423,042; 7,341,737; 7,303,761; and 6,787,155, all of which are hereby incorporated by reference in their entirety.

Cox-2 selective inhibitors. In one embodiment, a compound of the invention is administered before, concurrently or subsequent to administration of one or more Cox-2 inhibitors. Cox-2 inhibitors are a newer type of NSAID which are designed to be less harmful to the stomach. Etoricoxib is the Cox-2 selective inhibitor normally prescribed to treat gout. COX-2 inhibitors have been reported in the art and many chemical structures are known to produce inhibition of cyclooxygenase-2. COX-2 inhibitors are described, for example, in U.S. Pat. Nos. 5,616,601; 5,604,260; 5,593,994; 5,550,142; 5,516,752; 5,521,213; 5,474,995; 5,639,780; 5,604,253; 5,552,422; 5,510,368; 5,436,265; 5,409,944; and 5,130,311, all of which are hereby incorporated by reference in their entirety. Certain preferred COX-2 inhibitors include celecoxib (SC-58635), 5-bromo-2-(4-fluorophenyl)-3-(4-(methylsulfonyl)phenyl)-thiophene (DUP-697), flosulide (CUP-28238), meloxicam, 6-methoxy-2 naphthylacetic acid (6-MNA), MK-966 (also known as Vioxx), nabumetone (prodrug for 6-MNA), nimesulide, N-[2-(cyclohexyloxy)-4-nitrophenyl]-methanesulfonamide (NS-398), SC-5766, SC-58215, or 3-Formylamino-7-methylsulfonylamino-6-phenoxy-4H-1-benzopyran-1-one (T-614); or combinations thereof.

Corticosteroids. In one embodiment, a compound of the invention is administered before, concurrently or subsequent to administration of one or more corticosteroids. Corticosteroids are a type of steroid, and they sometimes are used in severe cases of gout. In some cases, a corticosteroid can be injected directly into the affected joint. The type of corticosteroid received will depend on the size of the affected joint. Suitable corticosteroids which may be used in combination with the compounds of the invention include, but are not limited to, methyl prednisolone, prednisolone, dexamethasone, fluticasone propionate, 6α,9α-difluoro-17-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17.alpha.-propionyloxy-androsta-1,4-diene-17β-carbothioic acid S-(2-oxo-tetrahydro-furan-3S-yl) ester, beclomethasone esters (e.g., the 17-propionate ester or the 17,21-dipropionate ester), budesonide, flunisolide, mometasone esters (e.g., the furoate ester), triamcinolone acetonide, rofleponide, ciclesonide, butixocort propionate, RPR-106541, and ST-126. Preferred corticosteroids include fluticasone propionate, 6α,9α-difluoro-11β-hydroxy-16α-Methyl-17α-[(-4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-di ene-17β-carbothioic acid S-fluoromethyl ester; and those disclosed in U.S. Pat. Nos. 7,288,536; 7,291,609; 7,157,433; 7,091,187; and 6,897,206, which are incorporated herein in their entirety.

Colchicine. In one embodiment, a compound of the invention is administered before, concurrently or subsequent to administration of colchicine. Colchicine inhibits uric acid crystal deposition, possibly by inhibiting oxidation of glucose and subsequent lactic acid production in leukocytes. Colchicine is available in tablet form and is usually taken every two to six hours. Colchicine can be administered in prodrug form.

Opioid agents. In one embodiment, a compound of the invention is administered before, concurrently or subsequent to administration of an opioid analgesic. Opioids act as agonists, interacting, with stereo specific and saturable binding sites in the brain and other tissues. Endogenous opioid-like peptides are present particularly in areas of the central nervous system that are presumed to be related to the perception of pain; to movement, mood and behavior, and to the regulation of neuroendocrinological functions. Opioid analgesics include, for example, morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine.

Cytokines. In one embodiment, a compound of the invention is administered before, concurrently or subsequent to administration of a modulator of one or more cytokines. Cytokines can be involved in an inflammatory response. Cytokines include, without limitation, BDNF, CREB pS133, CREB Total, DR-5, EGF, ENA-78, Eotaxin, Fatty Acid Binding Protein, FGF-basic, granulocyte colony-stimulating factor (G-CSF), GCP-2, Granulocyte-macrophage Colony-stimulating Factor GM-CSF (GM-CSF), growth-related oncogene-keratinocytes (GRO-KC), HGF, ICAM-1, IFN-alpha, IFN-gamma, the interleukins IL-10, IL-11, IL-12, IL-12 p40, IL-12 p40/p70, IL-12 p70, IL-13, IL-15, IL-16, IL-17, IL-18, IL-1alpha, IL-1beta, IL-1ra, IL-1ra/IL-1F3, IL-2, IL-3, IL-4, IL-5, IL-6, IL-7, IL-8, IL-9, interferon-inducible protein (10 IP-10), JE/MCP-1, keratinocytes (KC), KC/GROa, LIF, Lymphotacin, M-CSF, monocyte chemoattractant protein-1 (MCP-1), MCP-1 (MCAF), MCP-3, MCP-5, MDC, MIG, macrophage inflammatory (MIP-1 alpha), MIP-1 beta, MIP-1 gamma, MIP-2, MIP-3 beta, OSM, PDGF-BB, regulated upon activation, normal T cell expressed and secreted (RANTES), Rb (pT821), Rb (total), Rb pSpT249/252, Tau (pS214), Tau (pS396), Tau (total), Tissue Factor, tumor necrosis factor-alpha (TNF-alpha), TNF-beta, TNF-RI, TNF-RII, VCAM-1, and VEGF. In some embodiments, the cytokine is IL-12p70, IL-10, IL-1 alpha, IL-3, IL-12 p40, IL-1ra, IL-12, IL-6, IL-4, IL-18, IL-10, IL-5, eotaxin, IL-16, MIG, IL-8, IL-17, IL-7, IL-15, IL-13, IL-2R (soluble), IL-2, LIF/HILDA, IL-1 beta, Fas/CD95/Apo-1, or MCP-1. Modulation can comprise up or downregulating the biological action of the one or more cytokines. For example, gout can be treated by inhibiting IL-1. Inhibitors can comprise small molecules, peptides, proteins or the like. Alternately, serum uric acid levels can be decreased by administration of IL-6, or fragments, conjugates or mimetics thereof. In one embodiment, a compound of the invention is administered before, concurrently or subsequent to administration of a modulator of one or more IL-1 antagonists. In one embodiment, a compound of the invention is administered before, concurrently or subsequent to administration of a modulator of IL-6, or a fragment, conjugate or mimetic thereof.

One of skill in the art will appreciate that a combination therapy comprising a pharmaceutical composition of the present invention can further comprise a plurality of other pharmaceutically active agents as described herein. Any such combinations are within the scope of the present invention.

### (c) Cardiovascular and Related Disorders

As described above, hyperuricemia is associated with any number of cardiovascular disorders, including without limitation cardiovascular disease and other conditions, including hypertension, metabolic syndrome, hyperlipidemia, insulin resistance, coronary artery disease, peripheral artery disease cerebrovascular disease, vascular dementia, preeclampsia, heart disease, congestive heart failure, atherosclerosis and kidney disease. Furthermore, high levels of uric acid can predict the onset of hypertension, obesity, diabetes and kidney disease.

In some embodiments, a pharmaceutical composition of the present invention may be administered in combination with any other therapeutic agent and/or intervention that is commonly used for the treatment of cardiovascular or related disorders. Such agents include but are not limited to agents used to treat diabetes, including but not limited to agents that improve insulin sensitivity such as PPAR gamma ligands (thiazolidinedones, glitazones, troglitazones, rosiglitazone (Avandia), pioglitazone, stimulators of insulin secretion such as sulphonylureas (gliquidone, tolbutamide, glimepride, chlorpropamide, glipizide, glyburide, acetohexamide) and meglitinides (meglitinide, repaglinide, nateglinide) and agents that reduce liver production of glucose such as metformin. Such agents include but are not limited to agents used to treat vascular disease, including but not limited to endothelin receptor antagonists commonly used for the treatment of hypertension and other endothelial dysfunction-related disorders, such as bosentan, darusentan, enrasentan, tezosentan, atrasentan, ambrisentan sitaxsentan; smooth muscle relaxants such as PDE5 inhibitors (indirect-acting) and minoxidil (direct-acting); angiotensin converting enzyme (ACE) inhibitors such as captopril, enalapril, lisinopril, fosinopril, perindopril, quinaprit, trandolapril, benazepril, ramipril; angiotensin II receptor blockers such as irbesartan, losartan, valsartan, eprosartan, olmesartan, candesartan, telmisartan; beta blockers such as atenolol, metoprolol, nadolol, bisoprolol, pindolol, acebutolol, betaxolol, propranolol; diuretics such as thiazide, hydrochlorothiazide, furosemide, torsemide, metolazone; calcium channel blockers such as amlodipine, felodipine, nifedipine, verapamil, diltiazem; alpha receptor blockers doxazosin, terazosin, alfuzosin, tamsulosin; and central alpha agonists such as clonidine. Such agents include but are not limited to agents used to treat hyperlipidemia, including but not limited to agents that lower LDL such as statins (atovastatin, fluvastatin, lovastatin, pravastatin, rosuvastatin calcium, simvastatin) and nicotinic acid, agents that stimulate PPAR alpha such as fibrates, gemfibrozil, fenofibrate, bezafibrate, ciprofibrate, agents that bind and prevent readsorption of bile acids and reduce cholesterol levels such as bile acid sequestrants, cholestyramine and colestipol, and cholesterol absorption inhibitors. Such agents include those that reduce risk of heart attack, including COX-1 inhibitors including aspirin and NSAIDs, as described herein, or COX-2 inhibitors, also described herein.

In some embodiments, a pharmaceutical composition of the present invention may be administered in combination with any other therapeutic agent and/or intervention that is commonly used for the treatment of diabetes or related disorders. In such embodiments, combination therapy can be used for modulating (preventing the onset of the symptoms or complications associated with) diabetes (or treating, preventing or reducing the risk of developing, diabetes and its related symptoms, complications, and disorders), wherein the a pharmaceutical composition of the present invention can be effectively used in combination with, for example, biguanides (such as metformin); thiazolidinediones (such as ciglitazone, pioglitazone, troglitazone, and rosiglitazone); dipeptidyl-peptidase-4 (“DPP-IV”) inhibitors (such as vildagliptin and sitagliptin); glucagonlike peptide-1 (“GLP-1”) receptor agonists (such as exanatide) (or GLP-1 mimetics); PPAR gamma agonists or partial agonists; dual PPAR alpha, PPAR gamma agonists or partial agonists; dual PPAR delta, PPAR gamma agonists or partial agonists; pan PPAR agonists or partial agonists; dehydroepiandrosterone (also referred to as DHEA or its conjugated sulphate ester, DHEA-SO₄); antiglucocorticoids; TNF-alpha inhibitors; alpha-glucosidase inhibitors (such as acarbose, miglitol, and voglibose); sulfonylureas (such as chlorpropamide, tolbutamide, acetohexamide, tolazamide, glyburide, gliclazide, glynase, glimepiride, and glipizide); pramlintide (a synthetic analog of the human hormone amylin); other insulin secretogogues (such as repaglinide, gliquidone, and nateglinide); insulin (or insulin mimetics); glucagon receptor antagonists; gastric inhibitory peptide (“GIP”); or GIP mimetics.

In some embodiments, a pharmaceutical composition of the present invention may be administered in combination with any other therapeutic agent and/or intervention that is commonly used for the treatment of obesity or obesity-related disorders. In some embodiments, a pharmaceutical composition of the present invention can be used in combination with, for example, phenylpropanolamine, phenteramine; diethylpropion; mazindol; fenfluramine; dexfenfluramine; phentiramine, beta-3 adrenoceptor agonist agents; sibutramine; gastrointestinal lipase inhibitors (such as orlistat); and leptins. Other agents used in treating obesity or obesity-related disorders wherein the compounds of the present invention can be effectively used in combination with, for example, cannabinoid-1 (“CB-1”) receptor antagonists (such as rimonabant); PPAR delta agonists or partial agonists; dual PPAR alpha, PPAR delta agonists or partial agonists; dual PPAR delta, PPAR gamma agonists or partial agonists; pan PPAR agonists or partial agonists; neuropeptide Y; enterostatin; cholecytokinin; bombesin; amylin; histamine H3 receptors; dopamine D2 receptors; melanocyte stimulating hormone; corticotrophin releasing factor; galanin; and gamma amino butyric acid (GABA).

In some embodiments, a pharmaceutical composition of the present invention may be administered in combination with any other therapeutic agent and/or intervention that is commonly used for the treatment of hyperlipidemia and related complications, wherein the compounds of the present invention are used in combination with, for example, statins (such as atorvastatin, fluvastatin, lovastatin, pravastatin, and simvastatin), CETP inhibitors (such as torcetrapib); a cholesterol absorption inhibitor (such as ezetimibe); PPAR alpha agonists or partial agonists; PPAR delta agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; fenofibric acid derivatives (such as gemfibrozil, clofibrate, fenofibrate, and bezafibrate); bile acid-binding resins (such as colestipol or cholestyramine); nicotinic acid; probucol; betacarotene; vitamin E; or vitamin C.

In some embodiments, a pharmaceutical composition of the present invention may be administered in combination with any other therapeutic agent and/or intervention that is commonly used for the treatment of atherosclerosis, wherein a pharmaceutical composition of the present invention is administered in combination with one or more of the following active agents; an antihyperlipidemic agent; a plasma HDL-raising agent; an antihypercholesterolemic agent, such as a cholesterol biosynthesis inhibitor, e.g., an hydroxymethylglutaryl (HMG) CoA reductase inhibitor (also referred to as statins, such as lovastatin, simvastatin, pravastatin, fluvastatin, and atorvastatin); HMG-CoA synthase inhibitor; a squalene epoxidase inhibitor; or a squalene synthetase inhibitor (also known as squalene synthase inhibitor); an acyl-coenzyme A cholesterol acyltransferase (ACAT) inhibitor, such as melinamide; probucol; nicotinic acid and the salts thereof and niacinamide; a cholesterol absorption inhibitor, such as beta-sitosterol; a bile acid sequestrant anion exchange resin, such as cholestyramine, colestipol or dialkylaminoalkyl derivatives of a cross-linked dextran; LDL receptor inducer; fibrates, such as clofibrate, bezafibrate, fenofibrate, and gemfibrizol; vitamin B6 (also known as pyridoxine) and the pharmaceutically acceptable salts thereof, such as the HCl salt; vitamin B12 (also known as cyanocobalamin); vitamin 83 (also known as nicotinic acid and niacinamide); anti-oxidant vitamins, such as vitamin C and E and beta carotene; a beta-blocker; angiotensin II antagonist; on angiotensin converting enzyme inhibitor; PPAR alpha agonists or partial agonists; PPAR delta agonists or partial agonists; PPAR gamma agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; and a platelet aggregation inhibitor, such as fibrinogen receptor antagonists (i.e., glycoprotein IIb/IIIa fibrinogen receptor antagonists) and aspirin. As noted herein, the pharmaceutical compositions of the present invention can be administered in combination with more than one additional active agent, for example, a combination of a pharmaceutical composition of the present invention with an HMG-CoA reductase inhibitor (e.g., atorvastatin, fluvastatin, lovastatin, pravastatin, and simvastatin) and aspirin, or a combination of a pharmaceutical composition of the present invention with an HMG-CoA reductase inhibitor and a blocker.

Additionally, an effective amount of a pharmaceutical composition of the present invention and a therapeutically effective amount of one or more active agents selected from the group consisting of: an antihyperlipidemic agent; a plasma HDL-raising agent; an antihypercholesterolemic agent, such as a cholesterol biosynthesis inhibitor, for example, an HMG-CoA reductase inhibitor; an HMG-CoA synthase inhibitor; a squalene epoxidase inhibitor, or a squalene synthetase inhibitor (also known as squalene synthase inhibitor); an acyl-coenzyme A cholesterol acyltransferase inhibitor; probucol; nicotinic acid and the salts thereof; CETP inhibitors such as torcetrapib; a cholesterol absorption inhibitor such as ezetimibe; PPAR alpha agonists or partial agonists; PPAR delta agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; niacinamide; a cholesterol absorption inhibitor; a bile acid sequestrant anion exchange resin; a LDL receptor inducer; clofibrate, fenofibrate, and gemfibrozil; vitamin B6 and the pharmaceutically acceptable salts thereof; vitamin B12; an anti-oxidant vitamin; a beta-blocker; angiotensin II antagonist; an angiotensin converting enzyme inhibitor; a platelet aggregation inhibitor; a fibrinogen receptor antagonist; aspirin; phentiramines, beta-3 adrenergic receptor agonists; sulfonylureas, biguanides, alpha-glucosidase inhibitors, other insulin secretogogues, and insulin can be used together for the preparation of a pharmaceutical composition useful for treatments as described herein.

In some embodiments, a pharmaceutical composition of the present invention is administered in combination with any other therapeutic agent and/or intervention used for the treatment of metabolic syndrome (or treating metabolic syndrome and its related symptoms, complications and disorders), wherein the pharmaceutical compositions of the present invention can be effectively used in combination with, for example, the active agents discussed above for modulating or treating diabetes, obesity, hyperlipidemia, atherosclerosis, and/or their respective related symptoms, complications and disorders.

In a further embodiment, a pharmaceutical composition of the present invention can be administered in combination with halofenic acid, an ester of halofenic acid, or another prodrug halofenic acid, preferably with (−)-(4-chlorophenyl)-(3-trifluoromethylphenoxy)-acetic acid 2-acetylaminoethyl ester (metaglidasen).

### (d) Renal Disorders

In some embodiments, a pharmaceutical composition of the present invention may be administered in combination with any other therapeutic agent and/or intervention that is used for the treatment of renal or urological or related disorders, e.g., NO donors, calcium channel blockers, cholinergic modulators, alpha-adrenergic receptor antagonists, beta-adrenergic receptor agonists, phosphodiesterase inhibitors, cAMP-dependent protein kinase activators (e.g., cAMP mimetics), superoxide scavengers, potassium channel activators, estrogen-like compounds, testosterone-like compounds, benzodiazepines, adrenergic nerve inhibitors, antidiarrheal agents, HMG-CoA reductase inhibitors, smooth muscle relaxants, adenosine receptor modulators, adenylyl cyclase activators, endothelia receptor antagonists, bisphosphonates, cGMP-dependent protein kinase activators (e.g., cGMP mimetics). In some embodiments, the treatments for renal disorders comprise treatments for kidney stones. For example, the compound of the present invention can be given with muscle relaxants that assist in stone passage, including alpha adrenergic blocking agents such as Flomax, Uroxatral, terazosin or doxazosin. Pain of stones can be treated with nonsteroidal anti-inflammatories (NSAIDs) or opioids such as codeine or hydrocodone. NSAIDs and additional opioids are described herein. In some cases, thiazides, potassium citrate, magnesium citrate and allopurinol or a pharmaceutically acceptable salt thereof, are prescribed depending on the type of stone. For high urinary calcium, thiazides may be prescribed. Calgranulin may help prevent calcium oxalate kidney stone formation.

### (e) Dosing and Dosage Forms

The pharmaceutical compositions of the present invention may be administered by the usual routes and the dosage level depends upon the age, weight, conditions of the patient and the administration route. The compositions of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

In the methods of the present invention, the tranilast or a pharmaceutically acceptable salt thereof and allopurinol or a pharmaceutically acceptable salt thereof do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

Therapeutically effective dosages vary when the drugs are used in treatment combinations. Methods for experimentally determining therapeutically-effective dosages of drugs and other agents for use in combination treatment regimens are documented methodologies. One example of such a method is the use of metronomic dosing, i.e., providing more frequent, lower doses in order to minimize toxic side effects. Combination treatment further includes periodic treatments that start and stop at various times to assist with the clinical management of the patient.

In any case, multiple therapeutic agents can be administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies front more than zero weeks to less than four weeks. In addition, the combination methods, compositions and formulations are not to be limited to the use of only two agents; the use of multiple therapeutic combinations are also envisioned (e.g., 2, 3, 4 or more combinations).

It is understood that the dosage regimen to treat the condition(s) for which relief is sought, is optionally modified in accordance with a variety of factors. These factors include the condition from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form (e.g., combined in the same formulation) or in separate dosage forms (e.g., two or more different formulations) intended for substantially simultaneous administration. Simultaneous administration cart be by the same route or by different routes. The pharmaceutical agents that make up the combination therapy can optionally be administered sequentially, with either therapeutic agent being administered by a regimen calling for multi-step administration. The multi-step administration regimen optionally calls for sequential administration of the active agents or spaced-apart administration of the separate active agents. By “sequential” administration is meant a time difference of from seconds, minutes, hours or days between the two or more administration steps of the two or more active ingredients. The two or more agents may be administered in any order. The time period between the multiple administration steps may depend upon the properties of each pharmaceutical agent, such as potency, bioavailability, plasma half-life and kinetic profile of the pharmaceutical agent. Circadian variation of the target molecule concentrations are optionally used to determine the optimal dose interval.

In addition, methods of the present invention may be used in combination with procedures that provide additional or synergistic benefit to the patient. By way of example only, patients are expected to find therapeutic and/or prophylactic benefit in the methods of the present invention described herein, wherein such methods are combined with genetic testing to determine whether that individual is a carrier of a mutant gene that is correlated with a certain disease or condition.

Compositions of the present invention can be administered before, during or after the occurrence of a disease or condition, and the timing of administering the composition varies in some embodiments. Thus, for example, a composition of the present invention can be used as a prophylactic and can be administered continuously to subjects at risk of developing a condition or disease (e.g., inflammatory bowel disease, myocardial infarction or an autoimmune disorder) in order to prevent the occurrence of the disease or condition. Said subjects may be asymptomatic. Subjects with hyperuricemia as a sole diagnosis (or together with other indicators of disease) may be treated prophylactically for diseases and conditions described herein, e.g., those related to or arising from hyperuricemia. Compositions of the present invention can be administered to a subject during or as soon as possible after the onset of the symptoms. For example compositions of the present invention can be administered within the first 48 hours of the onset of the symptoms. In some embodiments the compositions can be administered within the first 6 hours of the onset of the symptoms or within 3 hours after the onset of the symptoms. The initial administration can be via any suitable route. Compositions comprising two or more active ingredients as disclosed herein can be administered as soon as is practicable after the onset of a disease or condition is detected or suspected, and for any length of time necessary for the treatment of the disease.

Tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain one or more active ingredients, sucrose as a sweetening agent, methyl and propylparabens as preservatives, a dye and flavoring such as cherry or orange flavor. In some embodiments, additional ingredients, for example, nonsteroidal anti-inflammatory drugs or colchicine, ingredients for treating other related indications, or inert substances such as artificial coloring agents are added. Of course, any material used in preparing any dosage unit form should be pharmaceutically pure and substantially non-toxic in the amounts employed. In addition, the one or more active ingredients may be incorporated into sustained-release preparations and formulations as described herein.

Examples of therapeutic compositions or modalities that can be combined with one or more compositions or methods of the invention are disclosed relevant portions of the following U.S. Patents or Patent Application Publications: Publication Nos. 2008/0221060; 2008/0249091; 2008/0233113; 2008/0200494; 2008/0114058; 2008/0076776; 2008/0038242; 2008/0188426; U.S. Pat. Nos. 7,452,867; 7,361,671; 7,232,812; 7,186,695; 7,030,119; 6,500,459; and 6,815,464.

Additional active agents that can be selected for combination therapy according to the present invention include structurally or functionally related therapeutic agents to those disclosed herein, e.g., without limitation, prodrugs, analogs, homologs, derivatives, isomers, mimetics, metabolic derivatives, secondary metabolites, esters, or salt forms. Analogs include compounds with substituted atoms or functional groups, transition state analogs or similar structure. Isomers include, without limitation, stereoisomers, enantiomers, geometrical isomers, cis-trans isomers, conformers, rotamers, tautomers, topoisomers or constitutional (structural) isomers. A structurally related compound could be a drug modified to improve pharmacological properties or processability. For a biological therapeutic agent, this could comprise a related peptide or immunotoxin. For example, a monoclonal antibody might be used in a combination therapy of the present invention. One of skill in the art will appreciate that combination therapy according to the present invention further comprises the monoclonal antibody conjugated to one or more toxic agents. Such antibody drug conjugates are well known in the art. See, e.g., U.S. Patent Publication No. 2008/0025989, filed Apr. 13, 2007 and entitled “Anti-Cd70 Antibody-Drug Conjugates and Their Use for the Treatment of Cancer and Immune Disorders.” Further, the present invention envisions the use of a peptide mimetic.

While the present invention has been described in conjunction with the specific embodiments set forth above, many alternatives, modifications and variations thereof will be apparent to those of ordinary skill in the art. Thus, for example, classes of known uricosuric or anti-inflammatory agents not recited above are within the scope of the invention, as are known but unrecited species of recited classes of uricosuric or anti-inflammatory agents. Similarly, the treatment of known but unrecited gouty conditions is within the scope of the invention. All such alternatives, modifications, and variations are intended to fall within the spirit and scope of the present invention.

The pharmaceutical compositions of the present invention may be administered once daily (QD), twice daily (BID), three times daily (TID) or four times per day (QID). In one embodiment, a pharmaceutical composition of the present invention is administered once daily (QD). In another embodiment, a pharmaceutical composition of the present invention is administered twice daily (BID).

### II. Methods of Use

### (a) Treatments of Hyperuricemic Disorders

A common target of treatment of gout aims to relieve pain and inflammation of the acute attack, and reduce the incidence of recurrent attacks. Dietary and pharmacological urate-lowering therapies principally aim to prevent or reverse uric acid crystal formation and clinical joint damage. Common approaches to the treatment of acute gout include corticosteroids, non-steroidal anti-inflammatory drugs (NSAIDs), and colchicine. The side effects of these drugs, particularly in the frail, elderly population who experience the highest incidence of acute gout, cart be serious. An approach to the prevention of recurrent gout is the use of a xanthine oxidase inhibitor, allopurinol or a pharmaceutically acceptable salt thereof. However, allopurinol or a pharmaceutically acceptable salt thereof can have serious side effects such as allopurinol or a pharmaceutically acceptable salt thereof hypersensitivity syndrome. See, e.g., Arellano et al. (1993) Ann Pharmacother 27:337-343.

Lowering the serum uric acid levels results in a reduction in the frequency of gout attacks. Maintaining serum uric acid levels below 6.0 mg/dL is commonly the target of treatment for chronic gout.

The pharmaceutical compositions and methods of the present invention are useful in reducing uric acid in a subject, such as a mammal. The term “mammal” as used herein can include humans, primates, livestock animals (e.g., sheep, pigs, cattle, horses, donkeys), laboratory test animals (e.g., mice, rabbits, rats, guinea pigs), companion animals (e.g., dogs, cats) and captive wild animals (e.g., foxes, kangaroos, deer). The mammal can be a human or a laboratory test animal. In some embodiments, the mammal is a human. Even in embodiments exemplified with respect to laboratory test animals, this should not be understood ht any way as limiting the application of the present invention to humans.

The term “subject” as used herein can be a mammal. In some embodiments, the term “subject” refers to a human. In some embodiments, the human is a patient. In some embodiments, the subject is known to suffer from a hyperuricemic disorder. For example, in some embodiments, the subject has uric acid crystal formation determined by aspiration of tophi or by aspiration of synovial fluid of an inflamed joint. In some embodiments, gouty conditions are determined by clinical criteria, e.g., podagra, tophi, or other joint pain and swellings, or an elevated serum uric acid level. Radiography techniques may also help determine whether a patient suffers from hyperuricemia, e.g., by showing evidence of joint damage or uric acid crystal formation. Such techniques include x-ray film, computed tomography (CT) scans, magnetic resonance imaging (MRI), DECT and ultrasound. In some embodiments, the subject is known to suffer from gout, e.g., by one or more prior occurrences of gouty attack. In some embodiments, the subject has severe gout. In same embodiments, the subject has refractory gout wherein prior art treatments have proven insufficient to control disease. In some embodiments, the subject has chronic gout. In some embodiments, the methods of the present invention are used to treat a patient with acute gout, e.g., presenting with a first attack comprising podagra.

In particular, the pharmaceutical compositions and methods of the present invention are useful in controlling the level of uric acid and uric acid crystal formation in a subject suffering from gout and ameliorating symptoms related to a high level of uric acid and uric acid crystal formation such as muscle spasm, localized swelling, inflammation, joint pains, muscle fatigue, stress feelings, and myocardial infraction. Crystal formation can be in one or more of the joints, under the skin, or in the kidneys. Some deposits may be so severe as to cause tophi.

Diseases associated with high levels of serum uric acid levels include, but are not limited to, gout, hyperuricemia, urinary lithiasis, hyperuricemic nephropathy, acute uric acid nephropathy and the like, especially gout and hyperuricemia.

An “effective amount” means an amount necessary at least partly to attain the desired response, or to delay the onset or inhibit progression or halt altogether, the onset or progression of a particular condition being treated. The amount varies depending upon the health and physical condition of the subject to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis can include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition. As described above, “treatment” as used herein includes prophylaxis.

Methods to determine serum uric acid levels are well known in the art and are often measured as part of a standard chemistry panel of blood serum samples. In various embodiments, the methods of the present invention lower serum uric acid levels in a subject by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more, as compared to serum uric acid levels in the subject prior to administering the methods of the present invention. In various embodiments, serum uric acid levels are decreased by at least between 5% to 50%, decreased by at least 25% to 75%, or decreased by at least 50% to 99%.

In some embodiments, the pharmaceutical compositions and methods of the present invention lower serum uric acid levels by 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10.0 mg/dL, or greater, as compared to serum uric acid levels in the subject prior to administering the methods of the present invention. In further embodiments, the pharmaceutical compositions and methods of the present invention lower serum uric acid levels by between 0.1-10.0 mg/dL, 0.5-6.0 mg/dL, 1.0-40 mg/dL or 1.5-2.5 mg/dL.

In one embodiment, the pharmaceutical compositions and methods of the present invention are used to treat a patient diagnosed with hyperuricemia, ameliorate symptoms associated with hyperuricemia, or prevent the onset of hyperuricemia by lowering or maintaining serum uric acid levels in a subject below 7.0 or 6.5 or 6.0 mg/dL, or lower. In some embodiments, the pharmaceutical compositions and methods are used to treat or prevent gout in a subject in need thereof. In some embodiments, the pharmaceutical compositions and methods are used to reduce the severity or number of gouty attacks in a subject in need thereof. In some embodiments, the pharmaceutical compositions and methods are used to reduce uric acid crystal formation in a subject in need thereof. For example, the pharmaceutical compositions and methods may ameliorate gout by reducing serum uric acid levels to an acceptable level wherein gouty attacks are less frequent or do not occur. Similarly, the pharmaceutical compositions and methods may ameliorate gouty symptoms by reducing serum uric acid levels to a level wherein adverse affects are no longer observed.

In some embodiments, one or more other therapeutic agents are administered in combination with a pharmaceutical composition of the invention to treat hyperuricemia or the effects thereof, e.g., gout. In some embodiments, the one or more other therapeutic agents comprise a xanthine oxidase inhibitor. In some embodiments, the xanthine oxidase inhibitor is febuxostat, oxypurinol, tisopurine, or an inositol. In some embodiments, the one or more other therapeutic agents comprise a uricosuric agent. In same embodiments, the uricosuric agent is probenecid, benzbromarone, sulfinpyrazone, guaifenesin, losartan, atorvastatin, amlodipine, adrenocorticotropic hormone (ACTH or corticotropin), or fenofibrate. In some embodiments, the one or more other therapeutic agents comprise a uricase enzyme, or a fragment or pegylated derivative thereof. In some embodiments, the uricase enzyme is rasburicase or pegloticase. In some embodiments, the one or more other therapeutic agents comprise cortisone. In some embodiments, the one or more other therapeutic agents comprise an anti-inflammatory agent. In some embodiments, the anti-inflammatory agent is a nonsteroidal anti-inflammatory drug (NSAID). In some embodiments, the NSAID is diclofenac, indomethacin, naproxen, sulindac, lumiracoxib or a Cox-2 selective inhibitor. In some embodiments, the Cox-2 selective inhibitor is etoricoxib, celecoxib (SC-58635), 5-bromo-2-(4-fluorophenyl)-3-(4-(methylsulfonyl)phenyl)-thiophene (DUP-697), flosulide (CGP-28238), meloxicam, 6-methoxy-2 naphthylacetic acid (6-MNA), MK-966 (Vioxx), nabumetone (6-MNA prodrug), nimesulide, N-[2-(cyclohexyloxy)-4-nitrophenyl]-methanesulfonamide (NS-398), SC-5766, SC-58215, or 3-Formylamino-7-methylsulfonylamino-6-phenoxy-4H-1-benzopyran-1-one (T-614). In some embodiments, the anti-inflammatory agent is a corticosteroid. In some embodiments, corticosteroid is methyl prednisolone, prednisolone, dexamethasone, fluticasone propionate, 6α,9α-difluoro-17-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17.alpha.-propionyloxy-androsta-1,4-diene-17β-carbothioic acid S-(2-oxo-tetrahydro-furan-3S-yl) ester, beclomethasone esters, the 17-propionate ester or the 17,21-dipropionate ester, budesonide, flunisolide, mometasone esters, the furoate ester, triamcinolone acetonide, rofleponide, ciclesonide, butixocort propionate, RPR-106541, ST-126, fluticasone propionate, 6α,9α-difluoro-11β-hydroxy-16α-methyl-17α-[(-4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, or 6α,9α-difluoro-17α-[(2-furanylcarbonyl)oxy]-11β-hydroxy-16α-methyl-3-oxo-androsta-1,4-diene-17β-carbothioic acid S-fluoromethyl ester. In some embodiments, the one or mare other therapeutic agents comprise Colchicine or a prodrug thereof. In some embodiments, the one or more other therapeutic agents comprise an opioid agent. In some embodiments, the opioid agent is morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine. In some embodiments, the one or more other therapeutic agents comprise an IL-1 antagonist. In some embodiments, the IL-1 antagonist is Canakinumab (ACZ885) or Rilonacept (Arcalyst). In some embodiments, the one or more other therapeutic agents comprise IL-6 or a fragment thereof.

The pharmaceutical compositions and methods of the present invention may be used to treat effects associated with hyperuricemia and gout. For example, the pharmaceutical compositions and methods of the present invention are used to treat pain associated with inflammation attributable to flares associated with gout attacks. In further embodiments, the pharmaceutical compositions and methods of the present invention may be used for treating inflammatory pain associated with gout.

Pain may be assessed using a measurement index. Indices that are useful in the methods of the present invention for the measurement of pain associated with hyperuricemia and gout include a visual analog scale (VAS), a Likert scale, categorical pain scales, descriptors, and the AUSCAN index, each of which is well known in the art.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain by choosing the spot on the line that best corresponds to the sensation of pain, where one end of the line corresponds to “no pain” (score of 0 cm) and the other end of the line corresponds to “unbearable pain” (score of 10 cm). This procedure provides a simple and rapid approach to obtaining quantitative information about how the patient is experiencing pain. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Liked scales and their use are described, e.g., in U.S. Pat. Nos. 6,623,040 and 6,766,319.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

Indices that are useful in the methods, compositions, and kits of the invention for the measurement of pain include the Pain Descriptor Scale (PDS), the Visual Analog Scale (VAS), the Verbal Descriptor Scales (VDS), the Numeric Pain Intensity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionnaire (MPQ), mean pain intensity (Descriptor Differential Scale), numeric pain scale (NPS) global evaluation score (GES) the Short-Form McGill Pain Questionnaire, the Minnesota Multiphasic Personality Inventory, the Pain Profile and Multidimensional Pain Inventory, the Child Heath Questionnaire, and the Child Assessment Questionnaire.

The pharmaceutical compositions and methods of the present invention are further useful for the treatment of inflammation and immune-related disorders. The administration of conventional urate lowering therapies leads to uric acid crystal remodeling that may result in inflammatory attacks and increase in painful flares, e.g., gout flares, during treatment. Moreover, patients suffering from gout suffer from chronic low level inflammation and the presence of inflammatory immune markers even in the absence of gouty attacks, as described herein. In some embodiments, the compositions and methods of the present invention provide anti-inflammatory and immune modulatory capabilities and are useful for reducing serum uric acid levels and concomitantly treating one or more of inflammation, inflammatory immune response, and pain associated therewith.

As described herein, a number of conditions are associated with hyperuricemia in addition to gout. These include a number of cardiovascular and renal complications. In some embodiments, the methods of the present invention are used to treat a renal disorder in a subject in need thereof. Renal disorders that can be treated with the methods of the invention include, but are not limited to, urinary lithiasis, hyperuricemic nephropathy, acute uric acid nephropathy, microalbuminuria, renal dysfunction, impaired glomerular filtration rate, and nephrolithiasis. In some embodiments, the methods of the present invention are used to treat kidney stones. The kidney stones may result directly from the deposition of uric acid, but may also result from the deposition of other materials, e.g., calcium oxalate or calcium phosphate, as sometimes observed in patients with hyperuricemia. In some embodiments, the methods of the present invention facilitate serum uric acid reduction in subjects suffering from renal insufficiency or chronic kidney disease. In some embodiments, treatments comprise administering a combination of a compound of the invention or pharmaceutically acceptable salt thereof and one or more therapeutic agents known to treat renal or urological disorders, including, but not limited to, a NO donor, a calcium channel blocker, a cholinergic modulator, an alpha-adrenergic receptor antagonist, a beta-adrenergic receptor agonist, a phosphodiesterase inhibitor, a cAMP-dependent protein kinase activator, a cAMP mimetic, a superoxide scavenger, a potassium channel activator, an estrogen-like compound, a testosterone-like compound, a benzodiazepine, adrenergic nerve inhibitor, an antidiarrheal agent, a HMG-CoA reductase inhibitor, a smooth muscle relaxant, a adenosine receptor modulator, an adenylyl cyclase activator, an endothelin receptor antagonist, a bisphosphonate, a cGMP-dependent protein kinase activator, a cGMP mimetic, an alpha adrenergic blocking agent, Flomax, Uroxatral, terazosin, doxazosin, a nonsteroidal anti-inflammatories, an opioid, codeine, hydrocodone, thiazide, potassium citrate, magnesium citrate, allopurinol or a pharmaceutically acceptable salt thereof, or calgranulin.

In some embodiments, the pharmaceutical compositions and methods of the invention are used to treat a cardiovascular disorder in a subject. Cardiovascular disorders that can be treated with the pharmaceutical compositions and methods of the invention include, but are not limited to, hypertension, myocardial infraction, metabolic syndrome, ischemic cardiac disease, coronary artery disease, cerebrovascular disease, vascular dementia, preeclampsia, heart disease, stroke, atherogenesis, thrombogenesis, atherosclerosis, inflammatory disease or peripheral, carotid, or coronary vascular disease. The pharmaceutical compositions and methods of the present invention are useful for treating or preventing any disorder associated with hyperuricemia, e.g., metabolic syndrome, hyperlipidemia, insulin resistance, diabetes, and adverse effects of obesity. In some embodiments, treatments comprise administering a combination of a compound of the invention or pharmaceutically acceptable salt thereof and one or more therapeutic agents known to treat a cardiovascular disorder, diabetes, or obesity, or complications thereof, including, but not limited to, glitazone, troglitazone, rosiglitazone (Avandia), pioglitazone, a sulphonylurea, gliquidone, tolbutamide, glimepride, chlorpropamide, glipizide, glyburide, acetohexamide, meglitinide, repaglinide, nateglinide, metformin, an endothelin receptor antagonist, bosentan, darusentan, enrasentan, tezosentan, atrasentan, ambrisentan sitaxsentan, a smooth muscle relaxant, a PDE5 inhibitor, minoxidil, an angiotensin converting enzyme (ACE) inhibitor, captopril, enalapril, lisinopril, fosinopril, perindopril, quinapril, trandolapril, benazepril, ramipril, a angiotensin II receptor blocker, irbesartan, losartan, valsartan, eprosartan, olmesartan, candesartan, telmisartan, a beta blocker, atenolol, metoprolol, nadolol, bisoprolol, pindolol, acebutolol, betaxolol, propranolol, a diuretic, thiazide, hydrochlorothiazide, furosemide, torsemide, metolazone, a calcium channel blocker, amlodipine, felodipine, nisoldipine, nifedipine, verapamil, diltiazem, a alpha receptor blocker, doxazosin, terazosin, alfuzosin, tamsulosin, a central alpha agonist, clonidine, a statin, atovastatin, fluvastatin, lovastatin, pravastatin, rosuvastatin calcium, simvastatin, nicotinic acid, a fibrate, gemfibrozil, fenofibrate, bezafibrate, ciprofibrate, a bile acid sequestrant, cholestyramine, colestipol, a cholesterol absorption inhibitor, a COX-1 inhibitor, aspirin, a NSAID, or a COX-2 inhibitor.

In some embodiments, the pharmaceutical compositions and methods of the present invention can further comprise measuring serum uric acid levels in the subject before and after administering tranilast or a pharmaceutically acceptable salt thereof and allopurinol or a pharmaceutically acceptable salt thereof, wherein a decrease in serum uric acid levels after the administering indicates an effective treatment. For example, a decrease below about 6 mg/dL indicates lack of hyperuricemia. But any decrease might be beneficial to the patient, e.g., by lowering serum uric acid level below a level where uric acid crystals form. Other diagnostic approaches can be used with the present invention to indicate a beneficial treatment. These include, without limitation, reduction of uric acid crystals as determined by aspiration, visible reduction of tophi, reduced or eliminated symptoms of gout, e.g., reduced inflammation or pain, as decreased uric acid in urine, as well as imaging of uric acid crystal burden.

### (b) Methods of Dosing and Treatment Regimens

One or more active ingredients are optionally used in the preparation of medicaments for the prophylactic and/or therapeutic treatment of hyperuricemic conditions (e.g., gout) or conditions that would benefit, at least in part, from amelioration. In addition, a method for treating any of the diseases or conditions described herein in a subject in need of such treatment, involves administration of tranilast or a pharmaceutically acceptable salt thereof and allopurinol or a pharmaceutically acceptable salt thereof, wherein the amount by weight of allopurinol or a pharmaceutically acceptable salt thereof is greater than the amount by weight of tranilast or a pharmaceutically acceptable salt thereof, either in a single pharmaceutical composition or in separate pharmaceutical compositions.

In the case wherein the patient's condition does not improve, upon the doctor's discretion the administration of one or more active ingredients are optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of one or more active ingredients are optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on along-term basis upon any recurrence of symptoms.

In unit dosage form, the formulations of the present invention is divided into unit doses containing appropriate quantities of tranilast or a pharmaceutically acceptable salt thereof and allopurinol or a pharmaceutically acceptable salt thereof. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The pharmaceutical compositions disclosed herein are contemplated to exhibit therapeutic activity when administered in an amount which can depend on the particular case. The variation in amount can depend, for example, on the human or animal and the active ingredients chosen. A broad range of doses can be applicable.

Dose titration or dose escalation protocols may be employed to determine the proper or optimal dose to administer to a subject. For example, dose titration or escalation studies may select for doses that improve efficacy or tolerability. Dose titration or escalation allows for the gradual adjusting of the dose administered until the desired effect is achieved. Dose titration gradually decreased the dosage administered while dose escalation gradually increases the dose administered. Methods of dose titration and escalation are well known in the art. As a non-limiting example, a mammal may be administered 300 mg/day tranilast or a pharmaceutically acceptable salt thereof and 400 mg/day of allopurinol or a pharmaceutically acceptable salt thereof every day and measured for serum uric acid levels on a daily basis. The dosage may be increased in increments of 5 mg/day on a weekly basis. The mammal may be monitored for a period of 12 weeks to find the desired dose.

Toxicity and therapeutic efficacy of such therapeutic regimens are optionally determined in cell cultures, experimental animals, or human studies, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. Active ingredients exhibiting high therapeutic indices are preferred. The data obtained from cell culture assays and animal studies are optionally used in formulating a range of dosage for use in human. The dosage of such active ingredients lies preferably within a range of circulating concentrations that include the ED50 with minimal toxicity. The dosage optionally varies within this range depending upon the dosage form employed and the route of administration utilized.

### III. Kits

Kits are contemplated for use herein. In one embodiment, a kit comprises a first dosage form comprising a pharmaceutical composition of the present invention in quantities sufficient to carry out the methods of the present invention, e.g., decreasing serum uric acid level, treating or preventing hyperuricemia, reducing pain or inflammation associated with hyperuricemia, treating or preventing gout, treating gouty symptoms, reducing the severity or number of gouty attacks, treating intercritical periods in gout patients, preventing, reducing or reversing uric acid crystal formation, treating a renal disorder, treating kidney stones, or treating a cardiovascular disorder. In some embodiments a kit is for a subject with a hyperuricemic disorder (e.g., gout) to use in the self-administration of the pharmaceutical composition, wherein the kit comprises a container housing a plurality of dosage forms and instructions for carrying out drug administration therewith. In one embodiment, a kit comprises a first dosage form comprising tranilast or a pharmaceutically acceptable salt thereof in one or more of the forms identified above (e.g., a tablet, capsule, pill, delayed release formulation) and at least a second dosage form comprising allopurinol or a pharmaceutically acceptable salt thereof, in quantities sufficient to carry out the methods of the present invention. The second dosage form, and any additional dosage forms (e.g., a third, fourth of fifth dosage form) can comprise any active ingredient disclosed herein for the treatment of a hyperuricemic disorder (e.g., gout). All dosage forms together can comprise a therapeutically effective amount of each compound for the treatment of a hyperuricemic disorder (e.g., gout). In some embodiments a kit is for a subject with a hyperuricemic disorder (e.g., gout) to use in the self-administration of at least one oral agent, wherein the kit comprises a container housing a plurality of said oral agents and instructions for carrying out drug administration therewith. At least one oral agent can comprise a combination of a therapeutically effective dose of tranilast or a pharmaceutically acceptable salt thereof and allopurinol or a pharmaceutically acceptable salt thereof.

### EXAMPLES

### Example 1

### Tranilast in Hyperuricemic Patients

The PRESTO (Prevention of Restenosis with Tranilast and its Outcomes) study was a multicenter study of ˜11,500 patients undergoing percutaneous transluminal coronary revascularization (PTCR) with or without stenting for single or multiple vessels over a 9-month period. The study compared the composite clinical event rate of death, myocardial infarction, or need for ischemia-driven target vessel revascularization in patients treated with Tranilast (300 and 450 mg twice daily) for 1 or 3 months versus placebo. Description of the study protocol and patient population can be found in Holmes et al., The PRESTO (Prevention of Restenosis with Tranilast and its Outcomes) protocol: A double-blind, placebo-controlled trial, Am Heart J 2000; 139:23-31; and Holmes et al., Results of Prevention of REStenosis with Tranilast and its Outcomes (PRESTO) Trial, Circulation. 2002; 106:1243, both of which are incorporated by reference herein in their entirety.

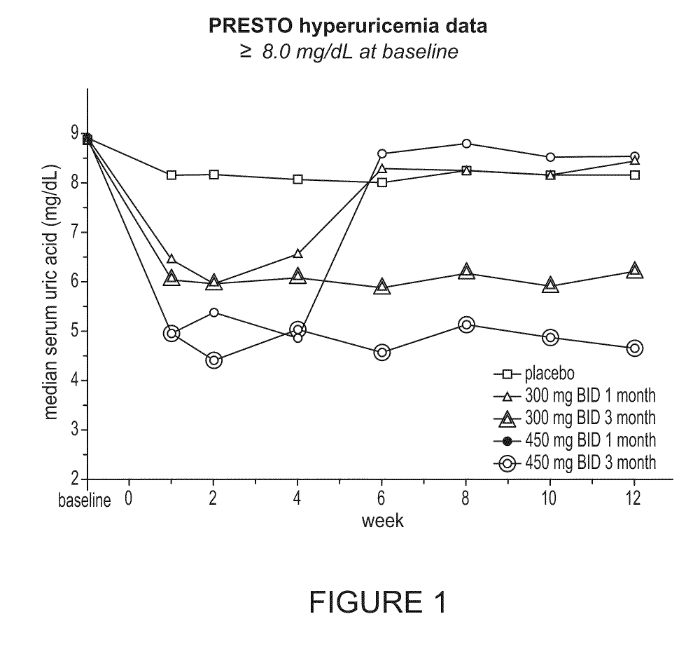

Gout was a contraindication for patient enrollment in the PRESTO trial. Nevertheless, 1100 patients enrolled in the trial were identified with baseline hyperuricemia defined as initial serum uric acid (sUA) levels greater than or equal to 7 mg/dL. Of these, approximately 300 participants had baseline serum uric acid levels greater than or equal to 8 mg/dL. In a prospective analysis of PRESTO data, serum uric acid levels were markedly decreased in patients treated with Tranilast, FIG. 1 shows the effects of Tranilast on uric acid levels in the hyperuricemic patients having uric acid baseline levels greater than or equal to 8 mg/dL. Data for FIG. 1 are shown in Table 2. Table 3 shows the demographic characteristics of patient population for FIG. 1 and Table 4. As indicated in FIG. 1 and Table 4, some patients were treated with placebo, some patients were treated with Tranilast for 4 weeks followed by placebo, and some patients were treated with Tranilast for 12 weeks.

TABLE 2
Change from Baseline in Uric Acid (mg/dL) over Time by Treatment Group

| | | | 1 M TRAN 300 mg/ | 1 M TRAN 450 mg/ | | |
|---|---|---|---|---|---|---|
| Visit | Statistic | 3 M PLACEBO | 2 M PLACEBO | 2 M PLACEBO | 3 M TRAN 300 mg | 3 M TRAN 450 mg |
| Baseline | N | 57 | 54 | 60 | 82 | 54 |
| | Mean (SD) | 8.86 (1.070) | 8.90 (0.949) | 8.82 (0.929) | 8.91 (1.446) | 8.81 (0.797) |
| | Median | 8.47 | 8.60 | 8.51 | 8.50 | 8.55 |
| | Min, Max | 8.02, 14.24 | 8.04, 12.80 | 8.07, 12.90 | 8.02, 19.38 | 8.07, 11.80 |
| Post-PTCR | N | 55 | 49 | 56 | 76 | 53 |
| | Mean (SD) | −0.80 (1.352) | −1.33 (1.220) | −2.00 (1.454) | −1.81 (1.966) | −2.16 (1.577) |
| | Median | −0.80 | −1.30 | −1.96 | −1.58 | −1.90 |
| | Min, Max | −8.44, 2.75 | −4.54, 2.03 | −5.21, 0.70 | −14.79, 1.90 | −7.34, 1.70 |
| Week 1 | N | 51 | 51 | 54 | 72 | 51 |
| | Mean (SD) | −0.95 (1.417) | −2.31 (1.839) | −3.66 (1.902) | −2.96 (2.394) | −3.84 (1.839) |
| | Median | −0.70 | −2.51 | −3.95 | −2.85 | −3.99 |
| | Min, Max | −8.56, 1.63 | −6.05, 2.70 | −8.00, 2.00 | −15.66, 3.14 | −8.79, 0.90 |
| Week 2 | N | 50 | 47 | 52 | 72 | 50 |
| | Mean (SD) | −0.90 (1.387) | −2.61 (2.149) | −3.43 (2.176) | −2.98 (2.224) | −4.05 (1.861) |
| | Median | −0.69 | −3.00 | −3.50 | −2.94 | −4.56 |
| | Min, Max | −7.77, 1.80 | −7.05, 1.80 | −8.40, 1.00 | −15.09, 2.30 | −8.72, 0.92 |
| Week 3 | N | 22 | 24 | 22 | 29 | 18 |
| | Mean (SD) | −0.93 (0.974) | −2.43 (2.143) | −4.19 (1.978) | −2.92 (1.946) | −2.84 (2.227) |
| | Median | −0.78 | −2.70 | −4.27 | −3.00 | −3.79 |
| | Min, Max | −3.90, 0.50 | −6.61, 3.50 | −7.90, −0.70 | −6.13, 1.92 | −5.79, 1.50 |
| Week 4 | N | 48 | 46 | 45 | 71 | 49 |
| | Mean (SD) | −0.94 (1.469) | −2.30 (1.953) | −3.52 (2.127) | −2.83 (2.212) | −3.70 (2.190) |
| | Median | −0.78 | −2.40 | −4.03 | −2.80 | −3.90 |
| | Min, Max | −8.29, 1.70 | −7.40, 2.60 | −7.65, 1.00 | −14.30, 1.98 | −8.40, 1.43 |
| Week 6 | N | 45 | 40 | 41 | 64 | 42 |
| | Mean (SD) | −0.84 (1.495) | −0.82 (1.416) | −0.29 (1.188) | −2.75 (2.755) | −3.89, (2.265) |
| | Median | −0.84 | −0.70 | −0.30 | −3.00 | −4.39 |
| | Min, Max | −8.24, 1.40 | −5.21, 2.40 | −3.20, 2.50 | −15.65, 8.50 | −8.69, 2.70 |
| Week 8 | N | 49 | 37 | 37 | 59 | 38 |
| | Mean (SD) | −0.93 (1.576) | −0.63 (1.371) | −0.49 (2.142) | −2.76 (2.451) | −3.65 (2.104) |
| | Median | −0.60 | −0.70 | −0.10 | −2.71 | −3.83 |
| | Min, Max | −9.10, 1.43 | −4.54, 1.55 | −9.70, 3.60 | −15.77, 1.70 | −7.16, 1.40 |
| Week 10 | N | 46 | 36 | 37 | 54 | 38 |
| | Mean (SD) | −0.98 (1.725) | −0.79 (1.195) | −0.46 (1.494) | −2.97 (2.490) | −3.73 (2.306) |
| | Median | −0.68 | −0.83 | −0.38 | −2.98 | −4.10 |
| | Min, Max | −8.64, 2.34 | −3.53, 2.10 | −4.40, 4.60 | −15.65, 2.30 | −8.60, 1.50 |
| Week 12 | N | 47 | 34 | 36 | 53 | 36 |
| | Mean (SD) | −0.88 (1.535) | −0.75 (1.500) | −0.56 (1.298) | −2.74 (2.634) | −3.79 (2.065) |
| | Median | −0.70 | −0.52 | −0.34 | −2.69 | −4.30 |
| | Min, Max | −8.66, 1.30 | −4.90, 1.50 | −3.50, 2.70 | −16.02, 3.60 | −8.12, 0.50 |
| Follow-Up | N | 17 | 18 | 21 | 26 | 17 |
| | Mean (SD) | −1.30 (2.413) | −0.64 (1.351) | −0.53 (1.249) | −0.00 (1.519) | −0.67 (1.277) |
| | Median | −0.70 | −0.86 | −0.40 | −0.13 | −0.70 |
| | Min, Max | −9.10, 1.00 | −3.30, 2.00 | −4.40, 1.20 | −3.00, 3.04 | −2.90, 1.80 |

TABLE 3
Demographic Characteristics

| | | | 1 M TRAN 300 mg/ | 1 M TRAN 450 mg/ | | | |
|---|---|---|---|---|---|---|---|
| | | 3 M PLACEBO | 2 M PLACEBO | 2 M PLACEBO | 3 M TRAN 300 mg | 3 M TRAN 450 mg | Total |
| | | (N = 64) | (N = 57) | (N = 65) | (N = 84) | (N = 59) | (N = 329) |
| Gender | Male | 51 (79.7%) | 43 (75.4%) | 54 (83.1%) | 68 (81.0%) | 48 (81.4%) | 264 (80.2%) |
| | Female | 13 (20.3%) | 14 (24.6%) | 11 (16.9%) | 16 (19.0%) | 11 (18.6%) | 65 (19.8%) |
| Age (years) | <45 | 7 (10.9%) | 5 (8.8%) | 7 (10.8%) | 5 (6.0%) | 4 (6.8%) | 28 (8.5%) |
| | 45-64 | 27 (42.2%) | 31 (54.4%) | 25 (38.5%) | 42 (50.0%) | 38 (64.4%) | 163 (49.5%) |
| | 65-69 | 11 (17.2%) | 9 (15.8%) | 13 (20.0%) | 14 (16.7%) | 8 (13.6%) | 55 (16.7%) |
| | 70-74 | 12 (18.8%) | 7 (12.3%) | 8 (12.3%) | 11 (13.1%) | 8 (13.6%) | 46 (14.0%) |
| | >=75 | 7 (10.9%) | 5 (8.8%) | 12 (18.5%) | 12 (14.3%) | 1 (1.7%) | 37 (11.2%) |
| Race | Caucasian | 62 (96.9%) | 46 (80.7%) | 57 (87.7%) | 78 (92.9%) | 58 (98.3%) | 301 (91.5%) |
| | Black | 0 (0.0%) | 6 (10.5%) | 4 (6.2%) | 4 (4.8%) | 1 (1.7%) | 15 (4.6%) |
| | Oriental | 0 (0.0%) | 0 (0.0%) | 0 (0.0%) | 0 (0.0%) | 0 (0.0%) | 0 (0.0%) |
| | Other | 2 (3.1%) | 5 (8.8%) | 4 (6.2%) | 2 (2.4%) | 0 (0.0%) | 13 (4.0%) |
| Age (years) | N | 64 | 57 | 65 | 84 | 59 | 329 |
| | MEAN | 60.656 | 60.561 | 62.523 | 61.845 | 58.475 | 60.921 |
| | MEDIAN | 62 | 62 | 65 | 62 | 59 | 62 |
| | STD | 11.680 | 11.220 | 11.937 | 11.175 | 9.183 | 11.129 |
| | Q1 | 52 | 51 | 57 | 54 | 50 | 52 |
| | Q3 | 70 | 69 | 72 | 71 | 66 | 70 |
| | MIN | 36 | 40 | 32 | 33 | 41 | 32 |
| | MAX | 82 | 84 | 80 | 83 | 75 | 84 |
| Weight (kg) | N | 64 | 57 | 65 | 84 | 59 | 329 |
| | MEAN | 91.233 | 94.610 | 90.729 | 90.376 | 93.227 | 91.857 |
| | MEDIAN | 90 | 92 | 90 | 88 | 93 | 90 |
| | STD | 16.965 | 21.202 | 16.658 | 18.576 | 17.107 | 18.105 |
| | Q1 | 77 | 81 | 80 | 77 | 80 | 79 |
| | Q3 | 101 | 101 | 101 | 103 | 105 | 102 |
| | MIN | 50 | 64 | 53 | 55 | 51 | 50 |
| | MAX | 129 | 161 | 134 | 153 | 136 | 161 |
| Height (cm) | N | 64 | 57 | 65 | 83 | 59 | 328 |
| | MEAN | 170.713 | 170.476 | 171.647 | 170.844 | 172.617 | 171.233 |
| | MEDIAN | 171 | 170 | 172 | 171 | 174 | 172 |
| | STD | 8.700 | 8.121 | 8.485 | 9.226 | 9.353 | 8.798 |
| | Q1 | 165 | 165 | 165 | 165 | 168 | 165 |
| | Q3 | 178 | 176 | 180 | 180 | 180 | 178 |
| | MIN | 152 | 150 | 152 | 147 | 150 | 147 |
| | MAX | 186 | 188 | 191 | 185 | 189 | 191 |

### Example 2

### Study A3008GT

Study A3008GT was a Phase 2, randomized, double-blind, 3-period, 3-treatment, balanced crossover study in otherwise healthy subjects with documented hyperuricemia and a screening sUA level ≧7.1 mg/dL to evaluate the effect of tranilast on allopurinol and oxipurinol PK and pharmacodynamics (PD) and to evaluate the effect of allopurinol on tranilast PK and PD.

The study evaluated co-administration of tranilast and allopurinol in patients with hyperuricemia. Subjects were randomized 1:1:1 in an initial 3-treatment crossover phase to 300 mg tranilast (T₃₀₀), 300 mg allopurinol (A₃₀₀), or a combination of A₃₀₀+T₃₀₀ (C₃₀₀). At end of the third period, patients were randomized 1:1 in a 2-treatment crossover phase of allopurinol 400 mg (A₄₀₀) or a combination of A₄₀₀+T₃₀₀ (C₄₀₀). Each period was 14 days in duration with 7 days active treatment orally once daily (Days 1-7), followed by a 7-day washout interval. Serum uric acid (sUA) levels were obtained each day of dosing and 24 hours after the last dose. Plasma tranilast, allopurinol, and oxypurinol concentrations were evaluated over the 24-hour interval after the last dose of each period. The primary objective was to compare percent change from baseline sUA of the combination compared to tranilast or allopurinol alone.

Subjects were screened for eligibility within 28 days before the start of dosing in Period 1. During each of the 3 periods, subjects checked-in to the Clinical Study Unit (CSU) on the morning of Day −2 and were domiciled until all study procedures were completed the morning of Day 8 (i.e., in the CSU for 9 full days and nights in each period, for a total of 27 full days and nights in the CSU to complete the study).

During Day −1 of each period, baseline PD testing (sUA and urinary uric acid (uUA) were initiated. Active treatment occurred on Days 1-7 of each period. Blood samples for the determination of sUA were collected on each day of dosing.

Urine was collected over 24 hours, at timed intervals, to evaluate uric acid excretion and creatinine clearance at baseline of each period (Day −1), on the first day of dosing in each period (Day 1), and the last day of dosing in each period (Day 7). Blood samples for the determination of trough plasma levels of tranilast and/or allopurinol and its metabolite oxipurinol were obtained on Days 6, 7, and 8 of each dosing period. Complete plasma concentration versus time profiles for the key PK measurements were evaluated over the 24-hour interval after the last dose(s) of tranilast and/or allopurinol in each period, with samples collected at 0, 0.5, 1, 1.5, 2, 3, 4, 5, 6, 8, 10, 12, 18, and 24 hours after the last dose of study drug on Day 7 of each period.

Safety laboratory testing (including liver and renal function assessments) and an evaluation of C-reactive protein (CRP), a marker of inflammation, were collected at screening, at baseline of each period (Day −1), the day after completion of dosing in each period (Day 8), and on Day 14 (±1 day) of Period 3. In addition, a biochemistry panel was tested on Days 3 and 5 to monitor liver and renal function.

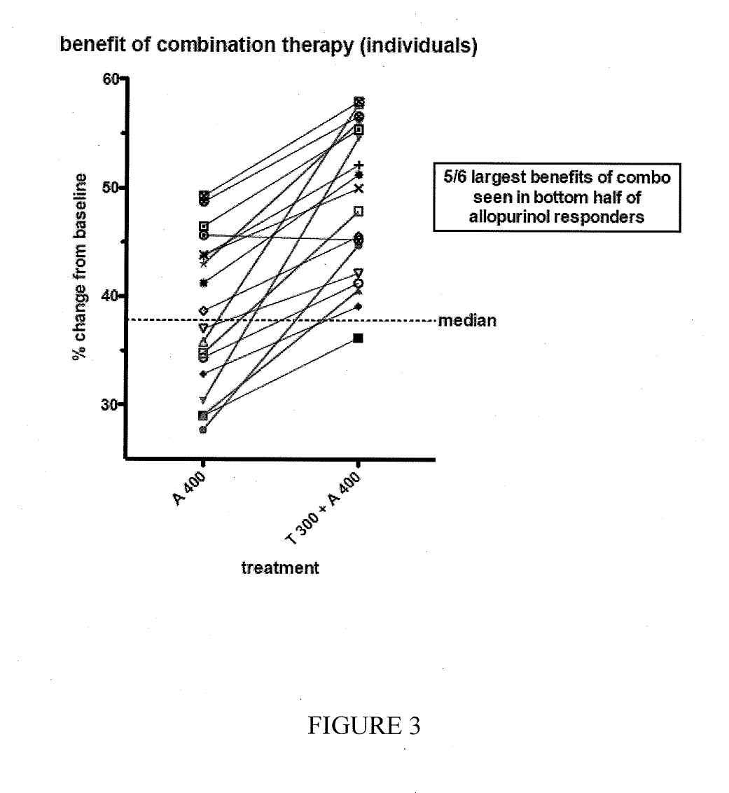

Twenty male patients were enrolled with mean age 43 years, BMI, 29.7 kg/m², and baseline sUA 8.1 mg/dL. Combination treatment resulted in greater percentage decrease in sUA than tranilast or allopurinol alone (see Table 4). Additionally, FIG. 3 shows the change is sUA levels in individual patients treated with a 400 mg dose of Allopurinol, as compared to a combination dose of 400 mg of Allopurinol and 300 mg Tranilast. As FIG. 3 shows, low Allopurinol responders as a group gained a marked benefit with the combination therapy, as a significant number of subjects with an sUA decrease below the median (37.85% median for allopurinol 400 mg group) when the group received Allopurinol monotherapy experienced decreases in sUA above the median (48.94% median for tranilast 300 mg+allopurinol 400 mg group) when the group received the combination treatment.

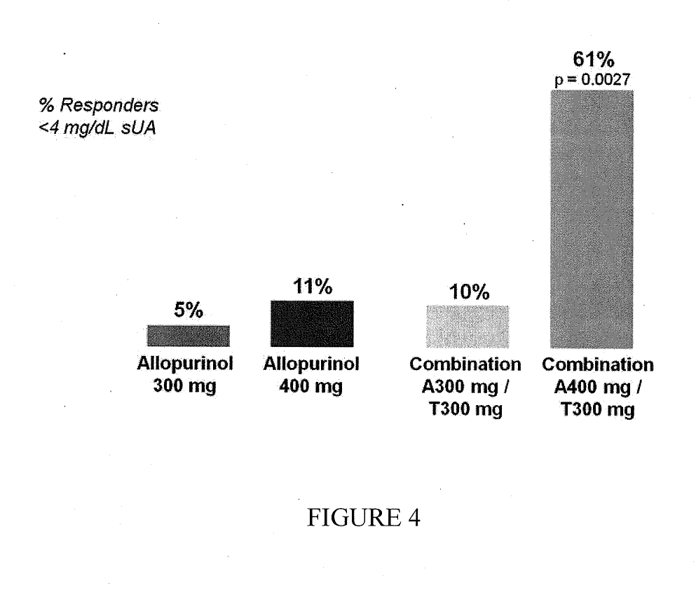

FIG. 4 shows the percentage of subjects in individual dosing groups with sUA level of <4 mg/dL after treatment with (1) Allopurinol 300 mg (n=19), (2) Allopurinol 400 mg (n=19), (3) Allopurinol 300 mg+Tranilast 300 mg (n=18) or (4) Allopurinol 400 mg+Tranilast 300 mg (n=18). As the figure demonstrates, dosing group (4), Allopurinol 400 mg+Tranilast 300 mg, provided the greatest percentage of patients with sUA levels <4 mg/dL.

The PK profiles (Cₘₐₓ and AUC₀₋₂₄) of allopurinol and tranilast were not affected by coadministration. However, surprisingly, the C₄₀₀ regimen was much more effective than any of the other dosing regimens, including the C₃₀₀ regimen, in reducing the sUA levels of patients below 4 mg/dL. With C₄₀₀, 61% of patients achieved sUA levels <4.0 mg/dL, which was significantly higher than the 11% achieved with C₃₀₀ or A₄₀₀ alone (p=0.0027).

TABLE 4
Dosing Regimens

| | | | C₃₀₀ | | C₄₀₀ |
|---|---|---|---|---|---|
| | T₃₀₀ | A₃₀₀ | (T₃₀₀ + A₃₀₀) | A₄₀₀ | (T₃₀₀ + A₄₀₀) |
| | n = 19 | n = 19 | n = 18 | n = 19 | n = 18 |
| Mean % sUA decrease from Baseline^{a} | 14.2% | 34.6% | 42.5%^{b} | 38.4% | 48.6%^{c} |
| Maximum % change | 33% | 55% | 56% | 49% | 58% |
| Minimum % change | 1% | 20% | 27% | 28% | 36% |
| Subjects <6 mg/dL sUA | 6/19 | 18/19 | 20/20 | 17/18 | 18/18 |
| | 26% | 95% | 100% | 94% | 100% |
| Subjects <5 mg/dL sUA | 1/19 | 6/19 | 16/20 | 13/18 | 17/18 |
| | 5% | 32% | 80% | 72% | 94% |
| Subjects <4 mg/dL sUA | 1/19 | 1/19 | 2/20 | 2/18 | 11/18 |
| | 5% | 5% | 10% | 11% | 61% |

^{a}24 h after Day 7 dose
^{b}P-value for LSMean difference between T₃₀₀ or A₃₀₀ and C₃₀₀: ≦0.0002.
^{c}P-value for LSMean difference between A₄₀₀ and C₄₀₀: <0.0001.

The PK profiles (Cₘₐₓ and AUC₀₋₂₄) allopurinol and tranilast or a pharmaceutically acceptable salt thereof were not affected by coadministration. Geometric means of combination treatment for tranilast or allopurinol were within “no-effect” limits of 80-125%. Tranilast resulted in at least a 25% reduction in plasma concentration of the active metabolite, oxipurinol.

### Example 3

### Air Pouch Model 1

The objective of this study was to evaluate the anti-inflammatory affects of tranilast versus a clinically active treatment for gout, colchicine, as well as a clinically active non-steroidal antiinflammatory drug, indomethacin. This evaluation was carried out in male Sprague-Dawley rats in a rodent model of gout. The animals were injected subcutaneously with 20 ml of sterile air, followed three days later by a supplemental injection with 20 ml of sterile air. Six days after the initial sterile air injection, the rats were injected intravenously with Evan's Blue and pretreated for thirty minutes with either a subcutaneous injection of colchicine (1 mg/kg) or indomethacin (5 mg/kg) or oral administration with either 200 mg/kg or 400 mg/kg of tranilast. After the pretreatment period, the rats were injected with 150 mg of monosodium urate (MSU) crystals (10 mg·ml) into the air pouch. Four hours later, the air pouch was injected with 5 ml heparinized saline and the entire contents of the air pouch removed, recording the total volume. The air pouch contents were evaluated for plasma extravasation and total white blood cell (WBC) count.

On day 0 the rats were anesthetized in a biological cabinet, the nape of the neck was cleansed with 70% isopropanol followed by iodine tincture (VEDCO, lot L021976). Twenty ml sterile (0.22 μm, Fisher Scientific, Cat. 09-720-3, lot R5SN25683) air was injected subcutaneously using a 23G×2 inch needle fixed to a 20 ml syringe. The rats were returned to routine housing. No adverse reactions were observed.

On day 3 the rats were anesthetized in a biological cabinet, the nape of the neck was cleansed with 70% isopropanol followed by iodine tincture (VEDCO, lot L021976). Twenty ml sterile (0.22 μm, Fisher Scientific, Cat. 09-720-3, lot R5SN25683) air was injected subcutaneously using a 23G×2 inch needle fixed to a 20 ml syringe. The rats were returned to routine housing. No adverse reactions were observed.

On day 6 the rats were weighed (Mettler, Model PE3000, SN: F69474) and sorted into six treatment groups of ten animals each, based upon average weight. The animals were either dosed subcutaneously with 1 ml/kg colchicine or indomethacin, or orally with either 4 ml/kg or 8 ml/kg tranilast or a pharmaceutically acceptable salt thereof, or 8 ml/kg vehicle. Immediately after test material administration, the rats were intravenously injected with 2 ml/kg Evans Blue,

TABLE 5
Treatment Groups

| | | Dose | |
|---|---|---|---|
| Group | Treatment | (mg/kg) | +MSU |
| 1 | Vehicle | N/A | YES |
| 2 | Colchicine | 1 | YES |
| 3 | Indomethacin | 5 | YES |
| 4 | tranilast | 200 | YES |
| 5 | tranilast | 400 | YES |
| 6 | Vehicle | N/A | NO |

Thirty minutes after test material administration, the rats were anesthetized and five groups were injected into the air pouch with 15 ml MSU using an 18G×1 inch needle fitted to a 20 ml syringe. The sixth group was injected with 15 ml saline. The rats were returned to their cages, no adverse affects were observed.

Four hours after MSU injection, the rats were anesthetized and 5 ml 10 U/ml heparinized saline was injected into the air pouch. The air pouch was gently massaged, the contents immediately removed using a 14G×1 inch needle fitted to a 6 ml syringe, and the exudate volume recorded. An aliquot of the exudate was transferred to heparin-treated microtainer tubes (Becton Dickinson, Cat. 365958, lot 8093666, exp. 06/09) for white blood cell (WBC) counting. The remainder of the exudate was centrifuged (Hermle Labrotechnik, Model Z200A, SN: 44060036), the supernatants removed and evaluated at OD₆₂₀ₙₘ (Spectronic Unicam, Model 4000 1/4, SN: 3SGD003006) for plasma extravasation.

TABLE 6
Average Exudate Volume (ml), WBC (cells/ml) and Total WBC
in Sample

| Group | Statistic | Exudate | WBC | Total WBC |
|---|---|---|---|---|
| 1 | Mean | 7.9 | 44150000 | 293500000 |
| | SD | 2.2 | 52211243 | 241424849 |
| 2 | Mean | 3.9 | 9215000 | 35131400 |
| | SD | 1.3 | 5110926 | 20711227 |
| 3 | Mean | 4.9 | 4160000 | 18520000 |
| | SD | 2.1 | 5556818 | 22309978 |
| 4 | Mean | 6.5 | 2524444 | 15925556 |
| | SD | 2.5 | 2236706 | 14881397 |
| 5 | Mean | 5.3 | 3144444 | 15162222 |
| | SD | 2.2 | 1473186 | 8878921 |
| 6 | Mean | 4.3 | 457000 | 1961600 |
| | SD | 0.2 | 299780 | 1242983 |

TABLE 7
Average Plasma Extravasation

| Group | Statistic | OD_{620 nm} |
|---|---|---|
| 1 | Mean | 1.612 |
| | SD | 0.309 |
| 2 | Mean | 0.776 |
| | SD | 0.239 |
| 3 | Mean | 0.505 |
| | SD | 0.203 |
| 4 | Mean | 0.697 |
| | SD | 0.324 |
| 5 | Mean | 0.626 |
| | SD | 0.221 |
| 6 | Mean | 0.004 |
| | SD | 0.015 |

### Conclusions

Effect of MSU Injection into Six-Day Air Pouch:

Four hours after MSU was injected into the six-day rat air pouch, 4.4×10⁷ WBC/ml were collected in 7.9 ml of exudate compared to 4.5×10⁵ WBC/ml in 4.3 ml exudate collected from animals which received an injection of saline instead of MSU. This was equivalent to a total WBC of 2.9×10⁸ in the MSU-treated rats relative to a total WBC of 2×10⁶ WBC in the saline control animals; approximately 150-fold increase in cell infiltration. The exudate collected from the MSU treated animals had an average OD₆₂₀ₙₘ of 1.612 relative to 0.004 OD in the saline treated rats, indicative of plasma extravasation associated with the inflammatory cell infiltration of the air pouch in response to MSU challenge.

Effect of Pretreatment with Colchicine:

Thirty minute pretreatment with 1 mg/kg colchicine, administered subcutaneously prior to MSU challenge, resulted in an 88% reduction in inflammatory cell infiltration associated with a 52% inhibition of plasma extravasation.

Effect of Pretreatment with Indomethacin:

Subcutaneous injection with 5 mg/kg indomethacin, thirty minutes prior to MSU challenge, yielded a 69% reduction of plasma extravasation accompanied by a 94% reduction in inflammatory cell infiltration into the air pouch.

Effect of Pretreatment with Tranilast or a Pharmaceutically Acceptable salt Thereof:

Oral pretreatment with tranilast or a pharmaceutically acceptable salt thereof, thirty minutes prior to injection of MSU into the air pouch, resulted in a 60% inhibition of plasma extravasation and 95% inhibition of white blood cell infiltration of the gouty air pouch, regardless of dose.

In sum, pretreatment with orally administered tranilast, regardless of dose, was as effective as colchicine in reducing the plasma extravasation and as effective as indomethacin in preventing inflammatory cell infiltration of the air pouch in response to MSU challenge.

### Example 4

### Air Pouch Model 2

The objective of this study was to evaluate the anti-inflammatory potency of Tranilast. This evaluation was carried out in male Sprague-Dawley rats in a rodent model of gout. The animals were injected subcutaneously with 20 ml of sterile air, followed three days later by a supplemental injection with 20 ml of sterile air. Six days after the initial sterile air injection, the rats were injected intravenously with Evan's Blue and pretreated for thirty minutes with oral administration of 25 mg/kg, 50 mg/kg, 100 mg/kg or 200 mg/kg tranilast. After the pretreatment period, the rats were injected with 150 mg of monosodium urate (MSU) crystals (10 mg/ml) into the air pouch. Four hours later, the air pouch was injected with 5 ml heparinized saline and the entire contents of the air pouch removed, recording the total volume. The air pouch contents were evaluated for plasma extravasation and total white blood cell (WBC) count.

On day 0 the rats were anesthetized in a biological cabinet, the nape of the neck was cleansed with 70% isopropanol followed by iodine tincture (VEDCO, lot L021976). Twenty ml sterile (0.22 μm, Millipore, Cat. SLGSV255F, lot R8J1494130, exp 07/2011) air was injected subcutaneously using a 23G×2 inch needle fixed to a 20 ml syringe. The rats were returned to routine housing. No adverse reactions were observed.

On day 3 the rats were anesthetized in a biological cabinet, the nape of the neck was cleansed with 70% isopropanol followed by iodine tincture (VEDCO, lot L021976). Twenty ml sterile (0.22 μm, Fisher Scientific, Cat. 09-720-3, lot R5SN25683) air was injected subcutaneously using a 23G×2 inch needle fixed to a 20 ml syringe. The rats were returned to routine housing. No adverse reactions were observed.

On day 6 the rats were weighed (Mettler, Model PE3000, SN: F69474) and injected intravenously injected with 2 ml/kg Evans Blue. The animals were dosed orally with 0.5 ml/kg, 1 ml/kg, 2, ml/kg or 4 ml/kg tranilast, or 4 ml/kg vehicle.

TABLE 8
Treatment Groups

| | | Dose | |
|---|---|---|---|
| Group | Treatment | (mg/kg) | +MSU |
| 1 | Vehicle | N/A | NO |
| 2 | Vehicle | N/A | YES |
| 3 | tranilast | 25 | YES |
| 4 | tranilast | 50 | YES |
| 5 | tranilast | 100 | YES |
| 6 | tranilast | 200 | YES |

Thirty minutes after test material administration, the rats were anesthetized and five groups (2-6) were injected into the air pouch with 15 ml MSU using a 19G×2 inch needle fitted to a 20 ml syringe. Group 1 was injected with 15 ml saline. The rats were returned to their cages, no adverse affects were observed.

Four hours after MSU/saline injection, the rats were anesthetized and 5 ml 10 U/ml heparinized saline was injected into the air pouch. The air pouch was gently massaged, the contents immediately removed using a 14G×1 inch needle fitted to a 6 ml syringe, and the exudate volume recorded. An aliquot of the exudate was transferred to heparin-treated microtainer tubes (Becton Dickinson, Cat. 365958, lot 9140110, expo 07/2010) for white blood cell (WBC) counting. The remainder of the exudate was centrifuged (Hermle Labrotechnik, Model Z200A, SN: 44060036), the supernatants removed and evaluated at OD₆₂₀ₙₘ (Spectronic Unicam, Model 4000 1/4, SN: 3SGD003006) for plasma extravasation.

TABLE 9
Average Exudate Volume (ml), WBC (cells/ml) and Total WBC
in Sample

| Group | Statistic | Exudate | WBC | Total WBC |
|---|---|---|---|---|
| 1 | Mean | 4.0 | 218000 | 867630 |
| | SD | 0.2 | 164303 | 643063 |
| 2 | Mean | 8.4 | 6343000 | 51644700 |
| | SD | 1.8 | 5071566 | 44960412 |
| 3 | Mean | 6.5 | 3196000 | 20550000 |
| | SD | 1.6 | 2751497 | 21688826 |
| 4 | Mean | 8.6 | 1557000 | 12476000 |
| | SD | 2.0 | 1773553 | 14460749 |
| 5 | Mean | 8.0 | 1566000 | 10629600 |
| | SD | 1.8 | 1538449 | 9417750 |
| 6 | Mean | 3.2 | 3516000 | 10305200 |
| | SD | 0.7 | 3842442 | 10637627 |

TABLE 10
Average Plasma Extravasation

| Group | Statistic | OD_{620 nm} |
|---|---|---|
| 1 | Mean | −0002 |
| | SD | 0.015 |
| 2 | Mean | 1.420 |
| | SD | 0.499 |
| 3 | Mean | 1.431 |
| | SD | 0.271 |
| 4 | Mean | 1.048 |
| | SD | 0.224 |
| 5 | Mean | 0.939 |
| | SD | 0.300 |
| 6 | Mean | 0.819 |
| | SD | 0.277 |

Effect of MSU Infection into Six-Day Air Pouch:

Four hours after MSU was injected into the six-day rat air pouch, 6.3×10⁶ WBC/ml were collected in 8.4 ml of exudate compared to 2.2×10⁵ WBC/ml in 4 ml exudate collected from animals which received an injection of saline instead of MSU. This was equivalent to a total WBC of 5.2×10⁷ in the MSU-treated rats relative to a total WBC of 8.7×10⁵ WBC in the saline control animals; approximately 60-fold increase in cell infiltration. The exudate collected from the MSU treated animals had an average OD₆₂₀ₙₘ of 1.420 relative to −0.002 OD in the saline treated rats, indicative of plasma extravasation associated with the inflammatory cell infiltration of the air pouch in response to MSU challenge.

Effect of Pretreatment with Tranilast or a Pharmaceutically Acceptable Salt Thereof:

Oral pretreatment with tranilast, thirty minutes prior to injection of MSU into the air pouch, resulted in a dose-dependent inhibition of the MSU-induced parameters. At the highest dose (200 mg/kg) a 42% inhibition of plasma extravasation and 80% inhibition of white blood cell infiltration of the gouty air pouch was recorded. The data shows a dose-response trend of decreasing extravasation and decreasing white blood cell infiltration with increasing dose of tranilast as indicated by a significant result (P=0.0013 and P=0.0001, respectively) when analyzed by an Analysis of Variance Test (ANOVA). A Sigmoidal dose-response (variable slope) analysis of the data provides and IC₅₀ effect of tranilast on plasma extravasation if 45 mg/kg and on white blood cell infiltration of 16 mg/kg.

In sum, pretreatment with orally administered tranilast, resulted in a dose-dependent inhibition of plasma extravasation and leukocyte infiltration of the air pouch in response to MSU challenge.

### Example 5

### Air Pouch Model 3

The objective of this study was to evaluate the anti-inflammatory potency of Tranilast. This evaluation was carried out in male Sprague-Dawley rats in a rodent model of gout. The animals were injected subcutaneously with 20 ml of sterile air, followed three days later by a supplemental injection with 20 ml of sterile air. Six days after the initial sterile air injection, the rats were injected intravenously with Evan's Blue and pretreated for thirty minutes with oral administration of 25 mg/kg, 50 mg/kg, 100 mg/kg, 200 mg/kg, or 300 mg/kg tranilast. After the pretreatment period, the rats were injected with 150 mg of monosodium urate (MSU) crystals (10 mg/ml) into the air pouch. Four hours later, the air pouch was injected with 5 ml heparinized saline and the entire contents of the air pouch removed, recording the total volume. The air pouch contents were evaluated for plasma extravasation and total white blood cell (WBC) count.

On day 0 the rats were anesthetized in a biological cabinet, the nape of the neck was cleansed with 70% isopropanol followed by iodine tincture (VEDCO). Twenty ml sterile (0.22 μm, Millipore, Cat. SLGSV255F) air was injected subcutaneously using a 23G×2 inch needle fixed to a 20 ml syringe. The rats were returned to routine housing. No adverse reactions were observed.

On day 3 the rats were anesthetized in a biological cabinet, the nape of the neck was cleansed with 70% isopropanol followed by iodine tincture (VEDCO). Twenty ml sterile (0.22 μm, Fisher Scientific, Cat. 09-720-3) air was injected subcutaneously using a 23G×2 inch needle fixed to a 20 ml syringe. The rats were returned to routine housing. No adverse reactions were observed.

On day 6 the rats were weighed (Mettler, Model PE3000, SN: F69474) and injected intravenously injected with 2 ml/kg Evans Blue. The animals were dosed orally with 0.5 ml/kg, 1 ml/kg, 2 ml/kg, 4 ml/kg tranilast or a pharmaceutically acceptable salt thereof, or 6 ml/kg tranilast or 6 ml/kg vehicle.

TABLE 11
Treatment Groups

| | | Dose | |
|---|---|---|---|
| Group | Treatment | (mg/kg) | +MSU |
| 1 | Vehicle | N/A | NO |
| 2 | Vehicle | N/A | YES |
| 3 | tranilast | 25 | YES |
| 4 | tranilast | 50 | YES |
| 5 | tranilast | 100 | YES |
| 6 | tranilast | 200 | YES |
| 7 | tranilast | 300 | YES |

Thirty minutes after test material administration, the rats were anesthetized and six groups (2-7) were injected into the air pouch with 15 ml MSU using a 19G×2 inch needle fitted to a 20 ml syringe. Group 1 was injected with 15 ml saline. The rats were returned to their cages, no adverse affects were observed.

Four hours after MSU/saline injection, the rats were anesthetized and 5 ml 10 U/ml heparinized saline was injected into the air pouch. The air pouch was gently massaged, the contents immediately removed using a 14G×1 inch needle fitted to a 6 ml syringe, and the exudate volume recorded. An aliquot of the exudate was transferred to heparin-treated, microtainer tubes (Becton Dickinson, Cat. 365958) for white blood cell (WBC) counting. The remainder of the exudate was centrifuged (Hermle Labrotechnik, Model Z200A, SN: 44060036), the supernatants removed and evaluated at OD₆₂₀ₙₘ (Spectronic Unicam, Model 4000 1/4, SN: 3SGD003006) for plasma extravasation.

TABLE 12
Average Exudate Volume (ml), WBC (cells/ml) and Total WBC
in Sample

| Group | Statistic | Exudate | WBC | Total WBC |
|---|---|---|---|---|
| 1 | Mean | 3.7 | 948000 | 3040400 |
| | SD | 0.6 | 2223690 | 6666456 |
| 2 | Mean | 8.4 | 36690000 | 302629000 |
| | SD | 1.8 | 32747466 | 284553989 |
| 3 | Mean | 6.9 | 31260000 | 204170000 |
| | SD | 1.9 | 26599549 | 174196900 |
| 4 | Mean | 4.8 | 18900000 | 96948000 |
| | SD | 1.4 | 12165616 | 77939099 |
| 5 | Mean | 6.4 | 27440000 | 145408000 |
| | SD | 3.0 | 28328988 | 129019202 |
| 6 | Mean | 6.2 | 12000000 | 71692000 |
| | SD | 1.8 | 11060641 | 68403802 |
| 7 | Mean | 5.6 | 5880000 | 29232000 |
| | SD | 2.6 | 5484686 | 24104106 |

TABLE 13
Average Plasma Extravasation

| Group | Statistic | OD_{620 nm} |
|---|---|---|
| 1 | Mean | 0.008 |
| | SD | 0.027 |
| 2 | Mean | 1.753 |
| | SD | 0.270 |
| 3 | Mean | 1.181 |
| | SD | 0.435 |
| 4 | Mean | 1.071 |
| | SD | 0.383 |
| 5 | Mean | 1.276 |
| | SD | 0.525 |
| 6 | Mean | 1.033 |
| | SD | 0.184 |
| 7 | Mean | 0.611 |
| | SD | 0.368 |

Effect of MSU Injection into Six-Day Air Pouch:

Four hours after MSU was injected into the six-day rat air pouch, 3.7×10⁷ WBC/ml were collected in 8.4 ml of exudate compared to 9.5×10⁵ WBC/ml in 3.7 ml exudate collected from animals which received an injection of saline instead of MSU. This was equivalent to a total WBC of 3.0×10⁸ in the MSU-treated rats relative to a total WBC of 3.0×10⁶ WBC in the saline control animals; a 100-fold increase in cell infiltration. The exudate collected from the MSU treated animals had an average OD₆₂₀ₙₘ of 1.753 relative to 0.008 OD in the saline treated rats, indicative of plasma extravasation associated with the inflammatory cell infiltration of the air pouch in response MSU challenge.

Effect of Pretreatment with Tranilast:

Oral pretreatment with tranilast, thirty minutes prior to injection of MSU into the air pouch, resulted in a dose-dependent inhibition of the MSU-induced parameters. At the highest dose (300 mg/kg) a 65% inhibition of plasma extravasation and 90% inhibition of white blood cell infiltration of the gouty air pouch was recorded. In sum, pretreatment with orally administered tranilast, resulted in a dose-dependent inhibition of plasma extravasation and leukocyte infiltration of the air pouch in response to MSU challenge.

### Example 6

### Air Pouch Model 4

The objective of this study was to evaluate the anti-inflammatory potency of Tranilast. This evaluation was carried out in male Sprague-Dawley rats in a rodent model of gout. The animals were injected subcutaneously with 20 ml of sterile air, followed three days later by a supplemental injection with 20 ml of sterile air. Six days after the initial sterile air injection, the rats were injected intravenously with Evan's Blue and pretreated for thirty minutes with oral administration of 3 mg/kg, 10 mg/kg, 30 mg/kg, 100 mg/kg, or 300 mg/kg tranilast. After the pretreatment period, the rats were injected, with 150 mg of monosodium urate (MSU) crystals (10 mg/ml) into the air pouch. Four hours later, the air pouch was injected, with 5 ml heparinized saline and the entire contents of the air pouch removed, recording the total volume. The air pouch contents were evaluated for plasma extravasation and total white blood cell (WBC) count.

On day 0 the rats were anesthetized in a biological cabinet, the nape of the neck was cleansed with 70% isopropanol followed by iodine tincture (VEDCO). Twenty ml sterile (0.22 μm, Millipore, Cat. SLGSV255F) air was injected subcutaneously using a 23G×2 inch needle fixed to a 20 ml syringe. The rats were returned, to routine housing. No adverse reactions were observed.

On day 3 the rats were anesthetized in a biological cabinet, the nape of the neck was cleansed with 70% isopropanol followed by iodine tincture (VEDCO). Twenty ml sterile (0.22 μm, Fisher Scientific, Cat. 09-720-3) air was injected subcutaneously using a 23G×2 inch needle fixed to a 20 ml syringe. The rats were returned to routine housing. No adverse reactions were observed.

On day 6 the rats were weighed (Mettler, Model PE3000, SN: F69474) and injected intravenously injected with 2 ml/kg Evans Blue. The animals were dosed orally with 0.06 ml/kg, 0.2 ml/kg, 0.6 ml/kg, 2 ml/kg, or 6 ml/kg tranilast or 6 ml/kg vehicle.

TABLE 14
Treatment Groups

| | | Dose | |
|---|---|---|---|
| Group | Treatment | (mg/kg) | +MSU |
| 1 | Vehicle | N/A | NO |
| 2 | Vehicle | N/A | YES |
| 3 | tranilast | 3 | YES |
| 4 | tranilast | 10 | YES |
| 5 | tranilast | 30 | YES |
| 6 | tranilast | 100 | YES |
| 7 | tranilast | 300 | YES |

Thirty minutes after test material administration, the rats were anesthetized and six groups (2-7) were injected into the air pouch with 15 MSU using a 19G×2 inch needle fitted to a 20 ml syringe. Group 1 was injected with 15 ml saline. The rats were returned to their cages, no adverse affects were observed.

Four hours after MSU/saline injection, the rats were anesthetized and 5 ml 10 U/ml heparinized saline was injected into the air pouch. The air pouch was gently massaged, the contents immediately removed using a 14G×1 inch needle fitted to a 6 ml syringe, and the exudate volume recorded. An aliquot of the exudate was transferred to heparin-treated microtainer tubes (Becton Dickinson, Cat. 365958) for white blood cell (WBC) counting. The remainder of the exudate was centrifuged (Hermle Labrotechnik, Model Z200A, SN: 44060036), the supernatants removed and evaluated at OD₆₂₀ₙₘ (Spectronic Unicam, Model 4000 1/4, SN: 3SGD003006) for plasma extravasation.

TABLE 15
Average Exudate Volume (ml), WBC (cells/ml) and Total WBC
in Sample

| | | | | Total |
|---|---|---|---|---|
| Group | Statistic | Exudate | WBC | WBC |
| 1 | Mean | 4.5 | 431000 | 1969900 |
| | SD | 0.6 | 381356 | 1795332 |
| 2 | Mean | 9.8 | 43360000 | 389336000 |
| | SD | 2.0 | 35267618 | 297225782 |
| 3 | Mean | 9.6 | 38510000 | 368228000 |
| | SD | 0.9 | 33458513 | 310223260 |
| 4 | Mean | 7.7 | 61200000 | 471436000 |
| | SD | 0.7 | 49824314 | 390429887 |
| 5 | Mean | 7.1 | 39650000 | 286935000 |
| | SD | 1.0 | 38622165 | 281432729 |
| 6 | Mean | 5.1 | 31740000 | 159916000 |
| | SD | 1.2 | 31730259 | 137413471 |
| 7 | Mean | 3.9 | 19466667 | 77451111 |
| | SD | 0.3 | 10047388 | 42189878 |

TABLE 16
Average Plasma Extravasation

| Group | Statistic | OD_{620 nm} |
|---|---|---|
| 1 | Mean | 0.028 |
| | SD | 0.007 |
| 2 | Mean | 1.845 |
| | SD | 0.468 |
| 3 | Mean | 1.940 |
| | SD | 0.365 |
| 4 | Mean | 1.662 |
| | SD | 0.498 |
| 5 | Mean | 1.089 |
| | SD | 0.418 |
| 6 | Mean | 1.180 |
| | SD | 0.483 |
| 7 | Mean | 0.613 |
| | SD | 0.314 |

Effect of MSU Infection into Six-Day Pouch:

Four hours after MSU was injected into the six-day rat air pouch, 4.3×10⁷ WBC/ml were collected in 9.8 ml of exudate compared to 4.3×10⁵ WBC/ml in 4.5 ml exudate collected from animals which received an injection of saline instead of MSU. This was equivalent to a total WBC of 3.9×10⁸ in the MSU-treated rats relative to a total WBC of 2×10⁶ WBC in the saline control animals; approximately 200-fold increase in cell infiltration. The exudate collected from the MSU treated animals had an average OD₆₂₀ₙₘ of 1.854 relative to 0.028 OD in the saline treated rats, indicative of plasma extravasation associated with the inflammatory cell infiltration of the air pouch in response to MSU challenge.

Effect of Pretreatment with Tranilast:

Oral pretreatment with tranilast, thirty minutes prior to injection of MSU into the air pouch, resulted in a dose-dependent inhibition of the MSU-induced parameters. At the highest dose (300 mg/kg) a 67% inhibition of plasma extravasation and 80% inhibition of white blood cell infiltration of the gouty air pouch was recorded. In sum, pretreatment with orally administered tranilast or a pharmaceutically acceptable salt thereof, resulted in a dose-dependent inhibition of plasma extravasation and leukocyte infiltration of the air pouch in response to MSU challenge.

### Example 7

### A Randomized, Double-Blind, Parallel Group Study to Evaluate the Safety and Efficacy of Tranilast in Combination with Allopurinol in Patients with Moderate to Severe Gout

This Example sets forth a phase 2 clinical study of the efficacy and safety of the combination of allopurinol and tranilast administered to patients with moderate to severe gout. In this study, the percent change from baseline in serum uric acid (sUA) levels following 4 weeks of once daily (QD) oral dosing with allopurinol and two doses of allopurinol in combination with tranilast be measured.

Secondary Objectives of this study include: (1) determination of the proportion of subjects whose sUA levels fall below 6.0 mg/dL following 4 weeks of dosing; (2) determination of the subjects' percent change from baseline in sUA levels following 24 weeks of dosing; (3) determination of the proportion of subjects whose sUA levels fall below 6.0 mg/dL following 24 weeks of dosing, and below 5.0 mg/dL, and below 4.0 mg/dL following 4 and 24 weeks of dosing; (4) assessment of the number of gout flares in subjects following 4 and 24 weeks of dosing: (5) assessment of the change in number of tophi in subjects following 24 weeks of dosing; (6) determination of the change from baseline in size of the largest tophus following 24 weeks of dosing; (7) assessment of the change in number of inflamed joints due to gouty arthritis following 4 and 24 weeks of dosing; (8) determination of the change in C-reactive protein (CRP) levels in subjects (a marker of inflammation) following 4 and 24 weeks of dosing; (9) determination of the subjects improvement in Quality of Life (QOL) and (10) assessment of the safety and tolerability of the drug combination,

### Study Design

The Phase 2 study is a multicenter, double-blind, randomized, active-comparator study to evaluate the safety and efficacy of tranilast in combination with allopurinol compared with allopurinol alone in subjects with hyperuricemia and moderate to severe gout, over a 4-week double-blind period.

At the end of Week 4, all subjects will have the option to continue in an openlabel extension (OLE) for an additional 20 weeks. Dosing will occur once daily at breakfast time. The study will be conducted at approximately 30 clinical sites in the United States (US). Approximately 90 subjects will be stratified based on serum uric acid level (<10.0 mg/dL, or ≧10.0 mg/dL) at Day 5 [or up to Day 2] and randomized at Baseline in a 1:1:1 ratio to receive either allopurinol 400 mg QD (A), the combination of tranilast 300 mg plus allopurinol 400 mg QD (C400), or the combination of tranilast 300 mg plus allopurinol 600 mg QD (C600).

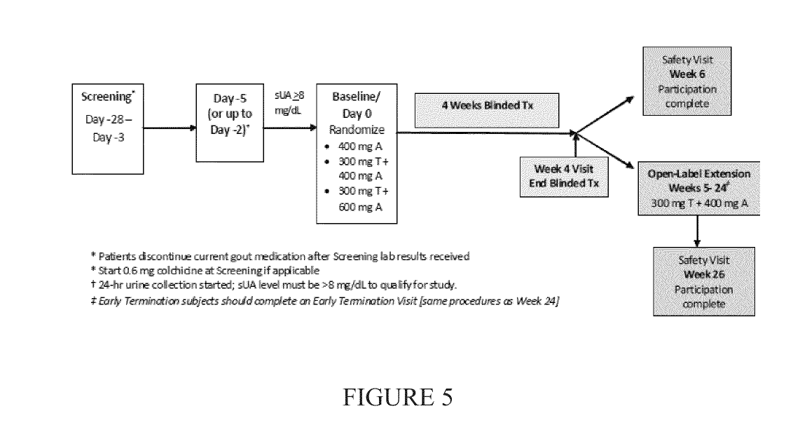

FIG. 5 provides a flowchart of the study design. All subjects will receive double-blind study treatment for 4 weeks. Dosing will occur at breakfast time. At the end of Week 4, all subjects will have the option to continue in an open-label extension (OLE) for an additional 20 weeks for a total dosing period of up to 24 weeks. All subjects in the OLE will receive a QD dose of tranilast 300 mg plus allopurinol 400 mg (C400).

During the double-blind 4-week period and the OLE, subjects will be monitored for safety, tolerability, and have efficacy assessments according to the Schedule of Assessments (Table 17),

TABLE 17
Schedule of Assessments.

| | | | | DOUBLE-BLIND DOSING | | | |
|---|---|---|---|---|---|---|---|
| | Visit # | | | | | | |
| | #1 | #2 | #3 | #4 | #5 | #6 | #7 |
| | Week | | | | | | |
| | | | Base- | | | | |
| | Screening^{a} | | line | W1 | W2 | W3 | W4ⁿ |
| | Day | | | | | | |
| | | | | D7 | | 21 | 28 |
| | D −28 to −3 | D −5 (or up to D −2) | D0 | (±2) | 14 (±2) | (±2) | (−3 to +2) |
| Informed Consent | X | | | | | | |
| Demographics | X | | | | | | |
| Medical History | X | | X^{b} | | | | |
| 12-lead ECG | X | | | | | | X |
| Inclusion/Exclusion | X | | X^{b} | | | | |
| Criteria | | | | | | | |
| Tophi Assessment^{c} | | | X | | | | X |
| Physical Exam^{d} | X | | X | | | | X |
| Colchicine or NSAID^{e} | X | X | X | X | X | X | X |
| Joint Count of # of | | | X | | | | X |
| swollen &tender joints | | | | | | | |
| QOL Questionnaires | | | X^{p} | | | | X |
| Vital Signs^{f} | X | | X | | X | | X |
| Randomization | | | X | | | | |
| Drug Dispensed by IWRS | | | X^{g} | | | | X^{h} |
| Drug Accountability | | | X | | X^{s} | | X |
| Biochemistryⁱ | X | X^{j} | X | X | X | X | X |
| Urinalysis & Hematology | X | | X^{r} | | | | X^{r} |
| Pregnancy test^{k} | X | | X | | | | |
| Abdominal Ultrasound | | | | | | | |
| 24-hour Urine Collection^{l} | | X^{l} | | | | | Xⁱ |
| Pharmacagenomic blood | | | X | | | | |
| sample | | | | | | | |
| Biomarkers^{m} | | | X | | | | X |
| Concomitant | X | | X | | X | | X |
| Medications & AEs | | | | | | | |

| | OPEN-LABEL EXTENSION (optional) | | | | | | | | | | |
|---|---|---|---|---|---|---|---|---|---|---|---|
| | Visit # | | | | | | | | | | |
| | #8 | #9 | #10 | #11 | #12 | #13 | #14 | #15 | #16 | #17 | #18 |
| | Week | | | | | | | | | | |
| | | | | | | | | | | W24/ | |
| | W5 | W6^{o} | W7 | W8 | W10 | W12 | W14 | W16 | W20 | ETⁿ | W26^{o} |
| | Day | | | | | | | | | | |
| | 35 | 42 | 49 | 56 | 70 | 84 | 98 | 112 | 140 | 168 | 182 |
| | (±2) | (±2) | (±2) | (±2) | (±2) | (±2) | (±2) | (±2) | (±2) | (±2) | (±2) |
| Informed Consent | | | | | | | | | | | |
| Demographics | | | | | | | | | | | |
| Medical History | | | | | | | | | | | |
| 12-lead ECG | | | | | | | | | | X | |
| Inclusion/Exclusion | | | | | | | | | | | |
| Criteria | | | | | | | | | | | |
| Tophi Assessment^{c} | | | | | | | | | | X | |
| Physical Exam^{d} | | | | | | | | | | X | X |
| Colchicine or NSAID^{e} | X | X | X | X | X | X | X | X | X | X | |
| Joint Count of # of | | | | | | | | | | X | |
| swollen &tender joints | | | | | | | | | | | |
| QOL Questionnaires | | | | | | | | | | X | |
| Vital Signs^{f} | | X | | X | X | X | X | X | X | X | X |
| Randomization | | | | | | | | | | | |
| Drug Dispensed by IWRS | | | | X^{h} | | X^{h} | | X^{h} | X^{h} | | |
| Drug Accountability | | X^{s} | | X | X^{s} | X | X^{s} | X | X | X | |
| Biochemistry^{l} | X | X | X | X | X | X | X | X | X | X^{p} | X |
| Urinalysis & Hematology | | | | X | | X | | X | | X^{r} | X |
| Pregnancy test^{k} | | | | | | | | | | X | X |
| Abdominal Ultrasound | | | | | | | | | | X^{q} | |
| 24-hour Urine Collection^{l} | | | | | | | | | | | |
| Pharmacagenomic blood | | | | | | | | | | | |
| sample | | | | | | | | | | | |
| Biomarkers^{m} | | | | | | | | | | X | |
| Concomitant | | X | | X | X | X | X | X | X | X | X |
| Medications & AEs | | | | | | | | | | | |

Footnotes to Table 17
^{a}The Screening Visit can occur between Day −28 and Day −3.
^{b}Medical history and Inclusion/Exclusion criteria will be updated prior to randomization.
^{c}A total count of tophi and a measurement of the largest tophus (identified at baseline) will be completed by the Investigator at Baseline, Week 4, and Week 24/ET.
^{d}Physical Examination including weight (and height at screening only).
^{e}Beginning with screening and during study treatment, subjects will be required to take 0.6 mg colchicine daily (preferred) or an NSAID, such as naproxen 375 mg twice daily, as prophylaxis to prevent gout flares.
^{f}Vital signs will be obtained after the subject has been seated quietly for 5 minutes, and collected prior to blood sample if scheduled for the same day.
^{g}Once-daily oral dosing taken at breakfast time of either 400 mg allopurinol, the combination of 300 mg tranilast and 400 mg allopurinol (C400), or the combination of 300 mg tranilast plus 600 mg allopurinol (C600).
^{h}All subjects enrolled in the OLE will receive once-daily dosing of a combination of C400.
ⁱIncluding serum uric acid and parameters for monitoring of hepatic and renal function.
^{j}Serum uric acid and creatinine only.
^{k}Urine pregnancy tests for women of childbearing potential only.
^{l}A 24 hr urine collection will begin one day before the Day −5 visit and end the following day on Day −5 (or up to Day −2) during Screening. A second collection will begin one day (~Day 27) before Week 4 (Day 28) visit and end on the day of the Week 4 visit. The total urine volume will be measured and an aliquot of urine taken for measurement of urinary creatinine and uric acid concentrations, The timed urine collection will be used to calculate the UUAE, FEUA, and CLcreat. In addition, “spot urine” samples may be taken to assess urine uric acid (uUA) on these days.
^{m}Blood samples will be taken for measurement of biomarkers associated with inflammation and gout.
ⁿET = Early termination; evaluations to be conducted for any subject who discontinues prematurely.
^{o}For all subjects, a Safety Visit will be required 2 weeks after the last dose of study drug (Week 6 or Week 26).
^{p}For subjects who discontinue the study due to elevated liver function tests, blood sample will be taken for assessment of HAV, HBV, HCV and related viruses.
^{q}Only required at the early termination visit for subjects who discontinue the study due to elevated liver function tests to assess the biliary tree patency.
^{r}Erythrocyte sedimentation rate (ESR) will be evaluated from the hematology sample at Baseline, Week 4, and Week 24/ET. Samples require special handling and shipping to the central lab.
^{s}Study drug is collected, drug accountability is performed, and drug is re-dispensed by the site at Weeks 2, 6, 10, and 14.

Serum uric acid (sUA) and safety laboratory testing (including liver and renal function assessments) will be collected at screening, Baseline (Day 0), and at each study visit (Weeks 1, 2, 3, 4, 5, 6, 7, 8, 10, 12, 14, 16, 20 and 24.). For assessment of urinary uric acid excretion (UUAE), fractional uric acid excretion (FEUA), and measurement of creatinine clearance (CLcreat), 24-hour urine collections will be required at Day 5 (or up to Day 2) prior to Baseline and Week 4. Subjects with tophi will have a tophus assessment (total count and measurement of the largest tophus) completed by the Investigator at Baseline, Week 4, and Week 24/ET. Subjects with tophi may have digital photographs of their tophi obtained by the Investigator at the specified timepoints as part of the study archives. In addition to CRP, additional markers of inflammation will be collected, at Baseline, Week 4, and Week 24/ET.

### Study Population

The study will be conducted at approximately 30 clinical sites in the United States (US). Approximately 90 subjects will be enrolled. Subjects will be aged ≧18 to ≦80 years. The subjects must be diagnosed with gout according to the 1977 ACR criteria and meet the protocol specified definition of moderate to severe gout, defined as 3 or more gout flares in the previous 12 months, or the presence of tophi or gouty arthritis. The study will aim to enroll at least 50% of subjects who have tophi. Subjects may be on current urate-lowering therapy for gout symptoms, and must have a sUA level ≧6.0 mg/dL assessed at screening to be considered for randomization. In order to be randomized, subjects will be required to discontinue arty urate-lowering therapy and have a sUA level ≧8.0 mg/dL after discontinuation of urate-lowering therapy prior to randomization,

### Duration of Participation

After a screening period of up to 4 weeks, subjects will be enrolled in the double-blind phase of the study for 4 weeks. Subjects will then have the option of continuing in an OLE for an additional 20 weeks for a total dosing duration of up to 24 weeks. All subjects will have a safety visit 2 weeks after their final dosing visit. Therefore, duration of participation, excluding screening and washout periods, will range from 6 weeks (4+2 weeks) for subjects who do not participate in the OLE to 26 weeks (24+2 weeks) for those who do participate in the OLE.

### Inclusion Criteria

Subjects who meet ALL of the following criteria will be considered for enrollment into this study:

1. Signed and dated written IRB-approved informed consent obtained from the subject in accordance with local regulations;
2. Male or female subject who is ≧18 to ≦80 years of age;
3. Moderate to severe gout, demonstrated by 3 or more gout flares in the previous 12 months, or the presence of at least one gout tophus or gouty arthritis;
4. Hyperuricemia with a sUA ≧8.0 mg/dL assessed at screening OR if on a urate-lowering agent, has a sUA ≧6.0 mg/dL assessed at screening and a sUA ≧8.0 mg/dL assessed after a washout period from urate-lowering therapy;
5. If sexually active, female subject of childbearing potential must agree to continue adequate contraception [i.e., hormonal (oral, depot, patch), IUD, or barrier and spermicide, or have a partner with a vasectomy] throughout the study and for at least 1 month following the last study visit;
6. In the opinion of the Investigator, the subject will be compliant and have a high probability of completing the study and all required procedures.

### Exclusion Criteria

Subjects who meet any of the following, criteria will be excluded from participation in this study;

1. Gout flare during screening or at baseline that has been resolved for fewer than seven (7) days prior to first treatment with study drug [subject can be rescreened once flare has resolved for more than one week as reported by the subject]; subjects with chronic joint inflammation due to gout (chronic gout flare) may be included;
2. Use of an investigational drug within 30 days or 5 half-lives (whichever is longer) prior to study drug administration;
3. Pregnant or nursing female;
4. Has any known hypersensitivity to any of the components of tranilast tablets;
5. Previous history of allopurinol hypersensitivity syndrome or hypersensitivity to any of the components of allopurinol tablets;
6. Known history of xanthinuria or uric acid urolithiasis;
7. Any blood laboratory test result at screening, except for sUA, that is considered clinically significant. The following will be considered clinically significantly:
ALT, AST, or alkaline phosphatase >1.5 times upper limit of normal (ULN) that persists upon repeat testing; or
Has an elevated total bilirubin (>1.5×ULN) that persists upon repeat testing; or
Cytopenia (to include any of the following: WBC <3.5×103/μL; Hgb <10 g/dL; platelets <100×103/μL; neutrophils absolute <1.5×103/μL; lymphocytes absolute <0.8×103/μL) that persists upon repeat testing
   8. Has a known history of Gilbert's syndrome;
   9. Has a history of clinically significant renal (estimated glomerular filtration rate [eGFR] <50 mL/min/1.73 m2) or hepatic dysfunction or disease;
   10. Has a history of alcohol or drug dependence as defined by DSM-IV-TR criteria within the last 2 years;
   11. Has a history or has a current, clinically significant major psychiatric disorder (e.g., major depressive disorder, psychosis, schizophrenia) according to the criteria of the Diagnostic and Statistical Manual of Mental Disorders, Fourth Edition (DSM-IV-TR) [Exception; subjects with depression that has been adequately controlled for at least 6 months may enroll in the study];
   12. Subject is planning or likely to require a surgical procedure during the study;
   13. For subjects who are to receive colchicine prophylaxis, receipt of drugs that may result in potentially significant interactions with colchicine, including inhibitors of P-glycoprotein, potent CYP3A4 inhibitors, and digoxin;
   14. Use of the following medications which may have clinically significant interactions with tranilast and/or allopurinol: (a) the oral hypoglycemic agents chlorpropamide, tolbutamide, glipizide, glimepiride, glyburide [=glibenclamide], repaglinide and nateglinide, rosiglitazone, and pioglitazone; (b) warfarin or dicoumarol; (c) phenyloin; and (d) thiazide diuretics, azathioprine, mercaptopurine, and cyclosporine;
   15. Presence of, or history of, cancer with the exception of completely excised, non-metastatic squamous cell and basal cell carcinoma of the skin;
   16. Coronary angioplasty, coronary stent placement, coronary bypass surgery, myocardial infarction, stroke, or transient ischemic attack within 12 months prior to screening;
   17. Any other acute or chronic medical condition that, in the opinion of the investigator, increases the risk to the subject or the likelihood that the subject will be unable to complete the study.

### Safety Evaluation

Safety assessments will be conducted throughout the study at protocol-specified timepoints (see Schedule of Assessments), and include: Adverse events (AEs); Vital sign measurements; Physical examinations; Electrocardiograms (ECGs); and Clinical laboratory tests (chemistry, hematology, urinalysis)

### Dose and Administration

Tranilast: 300 mg capsule taken orally QD.

Allopurinol: 400 mg capsule taken orally QD.

Allopurinol; 600 mg taken orally OD. The allopurinol 600 mg dose is administered by taking a 400 mg capsule and a 200 mg capsule.

Matching placebos will be used to maintain the blind, such that three (3) capsules per day will be administered for each treatment during the double-blind period.

Two (2) capsules per day will be administered during the open-label extension period. Subjects should take all capsules by mouth without opening them.

The tranilast, allopurinol, and placebo capsules will be indistinguishable in appearance.

### Concurrent Medications

Subjects may take acetaminophen at doses ≦1000 mg/day, low-dose aspirin (≦325 mg/day), and non-salicylate NSAIDs as needed, but may not take other medications that are frequently used to treat elevated sUA, such as probenecid, allopurinol (other than as study treatment), or febuxostat. Other treatments known to affect sUA including fenofibrate, losartan, citrate (≦500 mg/day), nicotinic acid and ascorbic acid (Vitamin C) may be used if taken at stable doses prior to screening and throughout the study without dose modification.

Subjects already taking colchicine or an NSAID for gout prophylaxis at the screening visit can continue this medication throughout the study. Subjects not on medication to prevent gout flares will initiate therapy at the screening visit with 0.6 mg Colcrys® (colchicine, USP), to be provided by Sponsor, orally once daily unless they are intolerant of colchicine; if intolerant, subjects should use an NSAID [such as naproxen 375 mg twice daily] as daily prophylaxis (not provided by Sponsor). Prophylaxis for gout flares should be taken through completion of the study. Should a subject's renal function begin to decline during the study, consideration should be given to changing gout flare prophylaxis from an NSAID to colchicine.

### Primary Efficacy Endpoint

The percent change from baseline in sUA levels following 4 weeks of treatment.

### Secondary Efficacy/Endpoints

Proportion of subjects whose sUA levels fall below 6.0 mg/dL following 4 weeks of treatment.

Proportion of subjects whose sUA levels are <6.0 mg/dL, <5.0 mg/dL, and <4.0 mg/dL following 4 and 24 weeks of treatment.

Percent change from baseline in sUA levels at Week 24.

Number of gout flares during the 4-week double-blind period, as well as the cumulative number of gout flares throughout the study.

Change from baseline at Week 4 and Week 24 in number of inflamed joints due to gouty arthritis.

Change from baseline at Week 4 and Week 24 in CRP levels, a marker of inflammation.

Change from baseline at Week 24 in the number of tophi.

Change from baseline at Week 24 in the size of the largest tophus.

Change from baseline at Week 4 and Week 24 in QOL.

Evaluation of safety and tolerability through periodic safety assessments.

While preferred embodiments of the present invention have been shown and described herein, it will be obvious to those skilled in the art that such embodiments are provided by way of example only.

Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising tranilast or a pharmaceutically acceptable salt or solvate thereof and allopurinol or a pharmaceutically acceptable salt or solvate thereof, wherein the amount by weight of said allopurinol or a pharmaceutically acceptable salt or solvate thereof in said composition is greater than the amount by weight of said tranilast or a pharmaceutically acceptable salt or solvate thereof in said composition.

2. The pharmaceutical composition according to claim 1, wherein said composition comprises about 50 mg to about 300 mg of tranilast or a pharmaceutically acceptable salt thereof.

3. The pharmaceutical composition according to claim 2, wherein said composition comprises about 300 mg to about 900 mg of allopurinol or a pharmaceutically acceptable salt thereof.

4. The pharmaceutical composition according to claim 3, wherein said composition comprises about 150 mg or about 300 mg of tranilast or a pharmaceutically acceptable salt thereof and about 400 mg or about 600 mg of allopurinol or a pharmaceutically acceptable salt thereof.

5. The pharmaceutical composition according to claim 4, wherein said composition comprises about 150 mg of tranilast or a pharmaceutically acceptable salt thereof and 400 mg of allopurinol or a pharmaceutically acceptable salt thereof.

6. The pharmaceutical composition according to claim 4, wherein said composition comprises about 300 mg of tranilast or a pharmaceutically acceptable salt thereof and about 400 mg of allopurinol or a pharmaceutically acceptable salt or solvate thereof.

7. The pharmaceutical composition according to claim 3, wherein said composition comprises about 300 mg of tranilast or a pharmaceutically acceptable salt thereof and about 600 mg of allopurinol or a pharmaceutically acceptable salt thereof.

8. The pharmaceutical composition according to claim 3, wherein said composition comprises about 300 mg of tranilast or a pharmaceutically acceptable salt thereof and about 800 mg of allopurinol or a pharmaceutically acceptable salt thereof.

9. The pharmaceutical composition according to claim 3, wherein said composition comprises about 300 mg of tranilast or a pharmaceutically acceptable salt thereof and about 900 mg of allopurinol or a pharmaceutically acceptable salt thereof.

10. The pharmaceutical composition according to claim 1, wherein the weight ratio of said allopurinol or pharmaceutically acceptable salt or solvate thereof to tranilast or pharmaceutically acceptable salt or solvate thereof is selected from the group consisting of at least 3.5:3, at least 4:3, at least 5:3, at least 2:1, at least 3:1, at least 4:1, at least 6:1, and at least 8:3.

11. The pharmaceutical composition according to claim 1, wherein the weight ratio of said allopurinol or pharmaceutically acceptable salt or solvate thereof to tranilast or pharmaceutically acceptable salt or solvate thereof is at least 4:3.

12. The pharmaceutical composition according to claim 1, wherein the weight ratio of said allopurinol or pharmaceutically acceptable salt or solvate thereof to tranilast or pharmaceutically acceptable salt or solvate thereof is at least 5:3.

13. The pharmaceutical composition according to claim 1, wherein the weight ratio of said allopurinol or pharmaceutically acceptable salt or solvate thereof to tranilast or pharmaceutically acceptable salt or solvate thereof is at least 2:1.

14. The pharmaceutical composition according to claim 1, wherein the weight ratio of said allopurinol or pharmaceutically acceptable salt or solvate thereof to tranilast or pharmaceutically acceptable salt or solvate thereof is at least 7:3.

15. The pharmaceutical composition according to claim 1, wherein the weight ratio of said allopurinol or pharmaceutically acceptable salt or solvate thereof to tranilast or pharmaceutically acceptable salt or solvate thereof is at least 8:3.

16. The pharmaceutical composition according to claim 1, wherein the weight ratio of said allopurinol or pharmaceutically acceptable salt or solvate thereof to tranilast or pharmaceutically acceptable salt or solvate thereof is about 4:3.

17. The pharmaceutical composition according to claim 1, wherein the weight ratio of said allopurinol or pharmaceutically acceptable salt or solvate thereof to tranilast or pharmaceutically acceptable salt or solvate thereof is about 2:1.

18. A method of treating a condition associated with an elevated serum uric acid level comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof.

19. The method according to claim 18, wherein said condition associated with an elevated serum uric acid level is gout.

20. The method according to claim 18, wherein said method comprises administering to said subject about 300 mg of tranilast or a pharmaceutically acceptable salt thereof.

21. The method according to claim 20, wherein said method comprises administering to said subject about 400 mg, about 500 mg, about 600 mg, about 700 mg, about 800 mg, or about 900 mg of allopurinol or a pharmaceutically acceptable salt thereof.

22. The method according to claim 21, wherein said method comprises administering to said subject about 400 mg of allopurinol or a pharmaceutically acceptable salt thereof.

23. The method according to claim 21, wherein said method comprises administering to said subject about 600 mg of allopurinol or a pharmaceutically acceptable salt thereof.

24. The method according to claim 21, wherein said method comprises administering to said subject about 800 mg of allopurinol or a pharmaceutically acceptable salt thereof.

25. The method according to claim 21, wherein said method comprises administering to said subject about 900 mg of allopurinol or a pharmaceutically acceptable salt thereof.

26. The method according to claim 18, wherein said method comprises administering to said subject about 150 mg of tranilast or a pharmaceutically acceptable salt thereof and about 300 mg, about 400 mg, about 500 mg, about 600 mg, about 700 mg, about 800 mg, or about 900 mg per day of allopurinol or a pharmaceutically acceptable salt thereof.

27. The method according to claim 18, wherein allopurinol or a pharmaceutically acceptable salt or solvate thereof is administered in multiple doses.

28. A method of decreasing serum uric acid in a subject in need thereof comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof.

29. The method according to claim 28, wherein said patient suffers from gout.

30. The method according to claim 28, wherein said method comprises administering to said subject about 300 mg of tranilast or a pharmaceutically acceptable salt thereof.

31. The method according to claim 30, wherein said method comprises administering to said subject about 400 mg, about 500 mg, about 600 mg, about 700 mg, about 800 mg, or about 900 mg of allopurinol or a pharmaceutically acceptable salt thereof.

32. The method according to claim 31, wherein said method comprises administering to said subject about 400 mg of allopurinol or a pharmaceutically acceptable salt thereof.

33. The method according to claim 28, wherein said method comprises administering to said subject about 150 mg of tranilast or a pharmaceutically acceptable salt thereof and about 300 mg, about 400 mg, about 500 mg, about 600 mg, about 700 mg, about 800 mg, or about 900 mg per day of allopurinol or a pharmaceutically acceptable salt thereof.

34. The method according to claim 33, wherein said method comprises administering to said subject about 400 mg of allopurinol or a pharmaceutically acceptable salt thereof.

35. The method according to claim 28, wherein allopurinol or a pharmaceutically acceptable salt or solvate thereof is administered in multiple doses.

36. A method of treating hyperuricemia in a subject comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof.

37. The method according to claim 36, wherein the hyperuricemia is associated with gout.

38. The method according to claim 37, wherein the gout is moderate to severe gout.

39. The method according to claim 36, wherein the subject suffers 3 or more gout flares in a period of about 12 months, or at least one gouty tophus, or gouty arthritis.

40. The method according to claim 36, wherein said method comprises administering to said subject about 300 mg of tranilast or a pharmaceutically acceptable salt thereof.

41. The method according to claim 40, wherein said method comprises administering to said subject about 400 mg, about 500 mg, about 600 mg, about 700 mg, about 800 mg, or about 900 mg of allopurinol or a pharmaceutically acceptable salt thereof.

42. The method according to claim 41, wherein said method comprises administering to said subject about 400 mg of allopurinol or a pharmaceutically acceptable salt thereof.

43. The method according to claim 41, wherein said method comprises administering to said subject about 600 mg of allopurinol or a pharmaceutically acceptable salt thereof.

44. The method according to claim 41, wherein said method comprises administering to said subject about 800 mg of allopurinol or a pharmaceutically acceptable salt thereof.

45. The method according to claim 41, wherein said method comprises administering to said subject about 900 mg of allopurinol or a pharmaceutically acceptable salt thereof.

46. The method according to claim 39, wherein said method comprises administering to said subject about 150 mg of tranilast or a pharmaceutically acceptable salt thereof and about 300 mg, about 400 mg, about 500 mg, about 600 mg, about 700 mg, about 800 mg, or about 900 mg per day of allopurinol or a pharmaceutically acceptable salt thereof.

47. The method according to claim 46, wherein said method comprises administering to said subject about 400 mg of allopurinol or a pharmaceutically acceptable salt thereof.

48. The method according to claim 41, wherein said method reduces said subject's serum uric acid level below 6.0 mg/dL.

49. The method according to claim 41, wherein said method reduces said subject's serum uric acid level below 5.5 mg/dL.

50. The method according to claim 41, wherein said method reduces said subject's serum uric acid level below 5.0 mg/dL.

51. The method according to claim 36, wherein said method comprises administering to said subject a pharmaceutical composition comprising tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof in said composition is greater than the amount by weight of said tranilast or pharmaceutically acceptable salt or solvate thereof in said composition.

52. The method according to claim 51, wherein said composition comprises about 150 mg or about 300 mg of tranilast or a pharmaceutically acceptable salt thereof.

53. The method according to claim 52, wherein said composition comprises about 400 mg, about 500 mg, about 600 mg, about 700 mg, about 800 mg, or about 900 mg of allopurinol or a pharmaceutically acceptable salt thereof.

54. The method according to claim 53, wherein said composition comprises about 300 mg of tranilast or a pharmaceutically acceptable salt thereof and about 400 mg of allopurinol or a pharmaceutically acceptable salt thereof.

55. The method according to claim 53, wherein said composition comprises about 300 mg of tranilast or a pharmaceutically acceptable salt thereof and about 500 mg of allopurinol or a pharmaceutically acceptable salt thereof.

56. The method according to claim 53, wherein said composition comprises about 300 mg of tranilast or a pharmaceutically acceptable salt thereof and about 600 mg of allopurinol or a pharmaceutically acceptable salt thereof.

57. The method according to claim 53, wherein said composition comprises about 300 mg of tranilast or a pharmaceutically acceptable salt thereof and about 800 mg of allopurinol or a pharmaceutically acceptable salt thereof.

58. The method according to claim 53, wherein said composition comprises about 300 mg of tranilast or a pharmaceutically acceptable salt thereof and about 900 mg of allopurinol or a pharmaceutically acceptable salt thereof.

59. The method according to claim 53, wherein said method reduces said subject's serum uric acid level below 6.0 mg/dL, below 5.0 mg/dL or below 4.0 mg/dL.

60. The method according to claim 36, wherein allopurinol or a pharmaceutically acceptable salt or solvate thereof is administered in multiple doses.

61. A method of treating gout in a subject comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof.

62. The method according to claim 61, wherein the gout is moderate to severe gout.

63. The method according to claim 61, wherein the subject suffers 3 or more gout flares in a period of about 12 months, or at least one gouty tophus, or gouty arthritis.

64. The method according to claim 61, wherein said method comprises administering to said subject about 300 mg of tranilast or a pharmaceutically acceptable salt thereof.

65. The method according to claim 64, wherein said method comprises administering to said subject about 400 mg, about 500 mg, about 600 mg, about 700 mg, about 800 mg, or about 900 mg of allopurinol or a pharmaceutically acceptable salt thereof.

66. The method according to claim 65, wherein said method comprises administering to said subject about 400 mg of allopurinol or a pharmaceutically acceptable salt thereof.

67. The method according to claim 65, wherein said method comprises administering to said subject about 600 mg of allopurinol or a pharmaceutically acceptable salt thereof.

68. The method according to claim 65, wherein said method comprises administering to said subject about 800 mg of allopurinol or a pharmaceutically acceptable salt thereof.

69. The method according to claim 65, wherein said method comprises administering to said subject about 900 mg of allopurinol or a pharmaceutically acceptable salt thereof.

70. The method according to claim 63, wherein said method comprises administering to said subject about 150 mg of tranilast or a pharmaceutically acceptable salt thereof and about 300 mg, about 400 mg, about 500 mg, about 600 mg, about 700 mg, about 800 mg, or about 900 mg per day of allopurinol or a pharmaceutically acceptable salt thereof.

71. The method according to claim 70, wherein said method comprises administering to said subject about 400 mg of allopurinol or a pharmaceutically acceptable salt thereof.

72. The method according to claim 65, wherein said method reduces said subject's serum uric acid level below 6.0 mg/dL.

73. The method according to claim 65, wherein said method reduces said subject's serum uric acid level below 5.5 mg/dL.

74. The method according to claim 65, wherein said method reduces said subject's serum uric acid level below 5.0 mg/dL.

75. The method according to claim 61, wherein said method comprises administering to said subject a pharmaceutical composition comprising tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof in said composition is greater than the amount by weight of said tranilast or pharmaceutically acceptable salt or solvate thereof in said composition.

76. The method according to claim 75, wherein said composition comprises about 150 mg or about 300 mg of tranilast or a pharmaceutically acceptable salt thereof.

77. The method according to claim 76, wherein said composition comprises about 400 mg, about 500 mg, about 600 mg, about 700 mg, about 800 mg, or about 900 mg of allopurinol or a pharmaceutically acceptable salt or solvate thereof.

78. The method according to claim 77, wherein said composition comprises about 300 mg of tranilast or a pharmaceutically acceptable salt thereof and about 400 mg of allopurinol or a pharmaceutically acceptable salt thereof.

79. The method according to claim 77, wherein said composition comprises about 300 mg of tranilast or a pharmaceutically acceptable salt thereof and about 600 mg of allopurinol or a pharmaceutically acceptable salt thereof.

80. The method according to claim 77, wherein said composition comprises about 300 mg of tranilast or a pharmaceutically acceptable salt thereof and about 700 mg of allopurinol or a pharmaceutically acceptable salt thereof.

81. The method according to claim 77, wherein said composition comprises about 300 mg of tranilast or a pharmaceutically acceptable salt thereof and about 800 mg of allopurinol or a pharmaceutically acceptable salt thereof.

82. The method according to claim 77, wherein said composition comprises about 300 mg of tranilast or a pharmaceutically acceptable salt thereof and about 900 mg of allopurinol or a pharmaceutically acceptable salt or solvate thereof.

83. The method according to claim 77, wherein said method reduces said subject's serum uric acid level below 6.0 mg/dL.

84. The method according to claim 77, wherein said method reduces said subject's serum uric acid level below 5.0 mg/dL.

85. The method according to claim 77, wherein said method reduces said subject's serum uric acid level below 4.0 mg/dL.

86. The method according to claim 61, wherein allopurinol or a pharmaceutically acceptable salt or solvate thereof is administered in multiple doses.

87. A method of treating hyperuricemia in a subject previously treated with a xanthine oxidase inhibitor alone, wherein the xanthine oxidase inhibitor treatment was insufficient to treat hyperuricemia, comprising administering to a subject in need thereof tranilast or a pharmaceutically acceptable salt or solvate thereof and allopurinol or a pharmaceutically acceptable salt or solvate thereof, wherein the amount by weight of said allopurinol or pharmaceutically acceptable salt or solvate thereof is greater than the amount by weight of said tranilast or pharmaceutically acceptable salt or solvate thereof.

88. The method according to claim 87, wherein the hyperuricemia is associated with gout.

89. The method according to claim 88, wherein the gout is moderate to severe gout.

90. The method according to claim 87, wherein the subject suffers 3 or more gout flares in a period of about 12 months, or at least one gouty tophus, or gouty arthritis.

91. The method according to claim 88, wherein said method comprises administering to said subject about 300 mg of tranilast or a pharmaceutically acceptable salt thereof.

92. The method according to claim 91, wherein said method comprises administering to said subject about 400 mg, about 500 mg, about 600 mg, about 700 mg, about 800 mg, or about 900 mg of allopurinol or a pharmaceutically acceptable salt thereof.

93. The method according to claim 92, wherein said method comprises administering to said subject about 400 mg of allopurinol or a pharmaceutically acceptable salt thereof.

94. The method according to claim 92, wherein said method comprises administering to said subject about 600 mg of allopurinol or a pharmaceutically acceptable salt thereof.

95. The method according to claim 92, wherein said method comprises administering to said subject about 800 mg of allopurinol or a pharmaceutically acceptable salt thereof.

96. The method according to claim 92, wherein said method comprises administering to said subject about 900 mg of allopurinol or a pharmaceutically acceptable salt thereof.

97. The method according to claim 90, wherein said method comprises administering to said subject about 150 mg of tranilast or a pharmaceutically acceptable salt thereof and about 300 mg, about 400 mg, about 500 mg, about 600 mg, about 700 mg, about 800 mg, or about 900 mg per day of allopurinol or a pharmaceutically acceptable salt thereof.

98. The method according to claim 97, wherein said method comprises administering to said subject about 400 mg of allopurinol or a pharmaceutically acceptable salt thereof.

99. The method according to claim 92, wherein said method reduces said subject's serum uric acid level below 6.0 mg/dL.

100. The method according to claim 92, wherein said method reduces said subject's serum uric acid level below 5.0 mg/dL.

101. The method according to claim 92, wherein said method reduces said subject's serum uric acid level below 4.0 mg/dL.

102. The method according to claim 87, wherein said composition comprises about 150 mg or about 300 mg of tranilast or a pharmaceutically acceptable salt thereof.

103. The method according to claim 102, wherein said composition comprises about 400 mg, about 500 mg, about 600 mg, about 700 mg, about 800 mg, or about 900 mg of allopurinol or a pharmaceutically acceptable salt or solvate thereof.

104. The method according to claim 103, wherein said composition comprises about 300 mg of tranilast or a pharmaceutically acceptable salt thereof and about 400 mg of allopurinol or a pharmaceutically acceptable salt thereof.

105. The method according to claim 103, wherein said composition comprises about 300 mg of tranilast or a pharmaceutically acceptable salt thereof and about 600 mg of allopurinol or a pharmaceutically acceptable salt thereof.

106. The method according to claim 103, wherein said composition comprises about 300 mg of tranilast or a pharmaceutically acceptable salt thereof and about 800 mg of allopurinol or a pharmaceutically acceptable salt thereof.

107. The method according to claim 103, wherein said composition comprises about 300 mg of tranilast or a pharmaceutically acceptable salt thereof and about 900 mg of allopurinol or a pharmaceutically acceptable salt or solvate thereof.

108. The method according to claim 87, wherein allopurinol or a pharmaceutically acceptable salt or solvate thereof is administered in multiple doses.

109. The method according to claim 87, wherein the xanthine oxidase inhibitor is allopurinol.
